(19) United States
(12) Patent Application Publication
(10) Pub. No.: US 2025/0049910 A1
(43) Pub. Date: Feb. 13, 2025

(54) Immunogenic Constructs And Vaccines For Use In The Prophylactic And Therapeutic Treatment Of Diseases Caused By SARS-CoV-2

(71) Applicants: Nykode Therapeutics ASA, Oslo (NO); Adaptive Biotechnologies Corporation, Seattle, WA (US)

(72) Inventors: Peter Ebert, Mountain View, CA (US); Agnete Fredriksen, Rælingen (NO); Mark Klinger, San Francisco, CA (US); Gunnstein Norheim, Oslo (NO); Edward Osborne, Seattle, WA (US); Monika Sekelja, Oslo (NO); Thomas Snyder, San Bruno, CA (US); Elisabeth Stubsrud, Oslo (NO)

(73) Assignees: Nykode Therapeutics ASA, Oslo (NO); Adaptive Biotechnologies Corporation, Seattle, WA (US)

(21) Appl. No.: 18/706,648

(22) PCT Filed: Nov. 3, 2022
(86) PCT No.: PCT/EP2022/080679

§ 371 Date: May 1, 2024

Related U.S. Application Data
(60) Provisional application No. 63/377,087, filed on Sep. 26, 2022.
(60) Provisional application No. 63/275,015, filed on Nov. 3, 2021.

(51) Int. Cl. A61K 39/215; C07K 14/005

(52) U.S. Cl. CPC A61K 39/215; C07K 14/005; C12N 2770/20022; C12N 2770/20034; C12N 2770/20052

(57) ABSTRACT

This invention relates to immunogenic constructs, such as polynucleotides, polypeptides and multimeric proteins and to antigenic units and to pharmaceutical compositions/vaccines comprising such immunogenic constructs or antigenic units, which are useful for the prophylactic and therapeutic treatment of diseases caused by severe acute respiratory coronavirus 2 (SARS-CoV-2), as well as methods for producing and using the immunogenic constructs, antigenic units and pharmaceutical compositions/vaccines.

### TECHNICAL FIELD

This invention relates to immunogenic constructs, such as polynucleotides, polypeptides and multimeric proteins and to antigenic units and to pharmaceutical compositions/vaccines comprising such immunogenic constructs or antigenic units, which are useful for the prophylactic and therapeutic treatment of diseases caused by severe acute respiratory coronavirus 2 (SARS-CoV-2), as well as methods for producing and using the immunogenic constructs, antigenic units and pharmaceutical compositions/vaccines.

### BACKGROUND

The coronavirus (CoV) is an enveloped, positive-sense single-stranded RNA virus that can cause diseases in a wide range of hosts, including humans. Four lineages (A-D) are commonly recognized and their genome, about 30 kb in length, is the largest found in RNA viruses and encodes more than 20 putative proteins, including four major structural proteins: spike (S), envelope (E), membrane (M), and nucleocapsid (N). The human seasonal CoVs are endemic throughout the world and cause respiratory tract infections that are typically mild and self-limiting. However, the outbreak of severe acute respiratory syndrome (SARS) in 2003 and Middle East respiratory syndrome (MERS) in 2012 that were caused by the infection of SARS-CoV and MERS-CoV, respectively, had led to higher mortality rates.

An outbreak of respiratory illness in Wuhan, China was reported by the WHO in January 2020, and found to be caused by a novel coronavirus, later renamed as severe acute respiratory syndrome coronavirus 2 (SARS-CoV-2). SARS-CoV-2 enters cells through interaction of the viral receptor binding domain (RBD) of the SARS-CoV-2 spike protein with angiotensin-converting enzyme 2 (ACE2) receptors, found on the surface of human nasopharyngeal, lung, and gut mucosa. Disease caused by this virus was termed coronavirus disease 2019 (COVID-19). Eventually, this novel viral infection has rapidly spread all over the world, leading to the declaration of the first-known coronavirus global pandemic by the World Health Organization (WHO) in March 2020. By September 2021 there have globally been reported over 218 million confirmed cases of COVID-19, including over 4.5 million deaths, to the WHO. To prevent transmission, hospitalisation, reduce severity and death related to COVID-19, an intense vaccine development effort was initiated in early 2020. Neutralizing antibodies targeting the RBD and other functional domains of the SARS-CoV-2 spike protein are a major route for achieving immunity and vaccine efficacy.

Based on prior experience from related coronaviruses and on the rationale that spike-specific antibodies constituted the majority of neutralizing antibodies in convalescent COVID-19 patients, vaccine technologies were adapted to induce spike-based protective immunity. Thus, many approved vaccines and vaccine candidates under development target the spike protein and its variants as the primary antigen. Multiple vaccines that were in clinical efficacy trials demonstrated to be highly immunogenic, safe and to protect against transmission, and/or to protect against severe disease and death. Multiple COVID-19 vaccines have therefore been authorized for use in various jurisdictions across the globe, and by September 2021, over 5.2 billion doses of COVID-19 vaccines have been administered globally.

The most successful vaccine platforms for SARS-CoV-2 are all based on inducing immunity to the spike glycoprotein, and include mRNA platforms, virus like particles with recombinant spike protein or, adenovirus vectors (Khoury et al 2021). These strategies have proven successful at eliciting neutralizing antibody responses against SARS-COV-2, and a number of studies have shown a correlation between virus neutralizing antibody levels and protection from symptomatic infection (Earle et al., 2021; Khoury et al., 2021). Neutralization titer is therefore a potential surrogate marker for protection against COVID-19. As neutralizing antibodies only explain part of the efficacy observed, similar studies to assess correlation between T cell response and efficacy are ongoing (Alter et al., 2021), possibly accelerating regulatory approval of T cell epitope-based vaccines.

A first major challenge for vaccine-based control of the pandemic per September 2021 is waning immunity and reduced efficacy of licensed or emergency authorized vaccines over time. Studies have shown that antibody levels induced by vaccines, as well as SARS-CoV-2 infection steadily decline 4-5 months after infection or vaccination; potentially affecting efficacy of the vaccine. The most recent data indicate that the protective effect is waning following two doses of e.g. mRNA vaccines authorized for use in the EU and USA. Several governments have therefore decided to recommend a booster dose at 6-8 months after completed regimen for high-risk population groups.

A second major challenge is the emergence of new variants of the SARS-COV-2 with the potential for increased transmission and/or reduced sensitivity to neutralizing antibodies generated by vaccines based on the 2020 prototype spike-based vaccines. Some of the acquired mutations (such as D614G in spike) enabled SARS-CoV-2 variants with increased transmissibility compared to the prototype Wuhan strain variant or escaping vaccine- or disease induced immune responses (such as E484K in spike), even in fully vaccinated individuals. The pandemic is therefore still ongoing and new variants of concern of the SARS-CoV-2 virus keep emerging; with the Delta variant currently dominating globally with greatest transmission capability and the Beta variant showing the greatest reduction in sensitivity to vaccine induced antibodies. With a higher transmissibility variant, a higher vaccine coverage is required to control the pandemic in the population, and global vaccine access is further delayed. Design of more broadly protective vaccines is possible based on close monitoring of variants, with particular attention to those that are spreading internationally, are associated with increased disease severity or demonstrate reduced sensitivity to neutralizing antibodies.

While the approved vaccines have had great impact in controlling the pandemic, and regular replacement of spike or RBD in vaccines with the most recent variant remains possible, there is still an urgent need for development of broadly protective, “universal” SARS-CoV-2 vaccines to control the ongoing pandemic, provide better protection against current and future variants of concern and secure global vaccine supply. Secondly, while care for patients who have COVID-19 has improved over time, there is also still an urgent need for virus-specific therapeutics for use in early phase of the disease progression.

Evidence of possible correlation between T cell immunity and the protection against COVID-19 has begun to arise (Bertoletti et al., 2021; Meyers et al., 2021). CD8+ cytotoxic T lymphocytes (CTLs) contribute to virus clearance from intracellular compartments inaccessible to neutralizing antibodies and support the SARS-CoV-2 antibody response by clearing virus-infected cells. They may also play a role in blocking transmission. Antigen-specific CD4+ T cells support B cells and CD8+ T cell generation, support of memory generation and indirect or direct cytotoxic activity.

Therefore, it is important to develop universal SARS-CoV-2 vaccines inducing specific T cell responses, with epitopes extending beyond those contained in the current spike based vaccines.

Thus, the present invention relates to vaccines comprising selected SARS-CoV-2 T cell epitopes which elicit a cellular immune response (T cell response) in human individuals to which they have been administered. The vaccines are thus useful for the prophylactic and therapeutic treatment of diseases caused by SARS-CoV-2.

### SUMMARY

In a first aspect, the disclosure relates to an immunogenic construct, the construct being: (i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii).

In some embodiments, the disclosure relates to an immunogenic construct, the construct being:
(i) a polynucleotide comprising a nucleotide sequence encoding a targeting unit that targets antigen presenting cells, a dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a dimeric protein consisting of two polypeptides as defined in (ii).

In another aspect, the disclosure relates to a vector comprising the polynucleotide as defined herein.

In yet another aspect, the disclosure relates to a host cell comprising the vector or polynucleotide as defined herein.

In yet another aspect, the disclosure relates to a polypeptide encoded by the nucleic acid sequence as defined herein.

In yet another aspect, the disclosure relates to a multimeric protein consisting of multiple polypeptides as defined herein. In some embodiments, the disclosure relates to a dimeric protein consisting of two polypeptides as defined herein.

In yet another aspect, the disclosure relates to the polynucleotide, the vector, the polypeptide or the multimeric protein as defined herein, for use as a medicament.

In yet another aspect, the disclosure relates to a pharmaceutical composition/vaccine comprising the polynucleotide, the vector, the polypeptide or the multimeric protein as defined herein, and a pharmaceutically acceptable carrier.

In yet another aspect, the disclosure relates to methods for preparing the pharmaceutical composition/vaccine and the use of the pharmaceutical composition/vaccine for the prophylactic or therapeutic treatment of a disease caused by SARS-CoV-2, such as by administering the pharmaceutical composition/vaccine to a subject in need of such prophylactic or therapeutic treatment.

The immunogenic construct or pharmaceutical composition/vaccine comprising such construct will, once administered to a subject elicit a rapid, strong and persistent T cell response and thus is useful as a prophylactic and therapeutic treatment of diseases caused by SARS-CoV-2.

In one embodiment, the pharmaceutical composition/vaccine may be administered to a human individual who has been previously vaccinated with a SARS-CoV-2 vaccine that targets the spike protein. Such individual may not be sufficiently protected against novel/future variants of the spike protein and the T cell vaccine of the invention will strengthen the individual's immune response to SARS-CoV-2 by boosting existing spike-specific T cell responses that may have been induced by the previous vaccine and add additional T cells specific for the non-spike antigens and thus fully utilizing CD4+/CD8+ T cell immunity in addition to neutralization response elicited by the previous vaccine.

In another embodiment, the pharmaceutical composition/vaccine may be administered to a human individual who has not yet been vaccinated with a SARS-CoV-2 vaccine and CD4+/CD8+ T cells provide protective immunity during SARS-CoV-2 infection.

The immunogenic construct of the invention is a vaccibody construct, i.e. a multimeric fusion protein consisting of multiple polypeptides (e.g. a dimeric protein consisting of two polypeptides), each polypeptide comprising a targeting unit, which targets antigen-presenting cells, a multimerization unit and an antigenic unit and which, after administration to a subject, has shown to be efficient in generating an immune response against the antigens or epitopes comprised in the antigenic unit. Vaccibody constructs have previously been suggested as vaccines against SARS-CoV-2 infection, see for example PCT/EP2021/061602, the content of which is included herein by reference and G. Norheim et al., bioRvix 2020, doi: https://doi.org/10.1101/2020.12.08.416875.

The construct disclosed herein may be administered to a subject, e.g. a human individual, in the form of a polynucleotide (e.g. a DNA plasmid) comprising a nucleotide sequence encoding the polypeptide. After administration to host cells, e.g. muscle cells, the polypeptide is expressed which, due to the multimerization unit, such as dimerization unit, forms a multimeric fusion protein, such as a dimer.

The immunogenic construct of the invention may be used in a vaccine, i.e. a pharmaceutical composition comprising the construct of the invention and a pharmaceutically acceptable carrier, for use in the prophylactic or therapeutic treatment of diseases caused by SARS-CoV-2, by administering the vaccine to a subject, i.e. a human individual.

The antigenic unit described herein is a further aspect of the disclosure. Thus, in such further aspect, the disclosure refers to (i) a polynucleotide comprising a nucleotide sequence encoding an antigenic unit comprising at least 77 SARS-CoV-2 T cell epitopes with amino acid sequences having at least 73% sequence identity to the amino acid sequences SEQ ID NOs: 1-77; or (ii) a polypeptide encoded by the nucleic acid sequence defined in (i).

Such antigenic unit (in the form of the polynucleotide or polypeptide) may be used in a pharmaceutical composition/vaccine comprising a pharmaceutically acceptable carrier on its own, i.e. without the targeting unit and the multimerization unit being present in the polynucleotide/polypeptide.

On its own, the antigenic unit may be used in the form of a polynucleotide as described herein, e.g. a DNA or an RNA, including genomic DNA, cDNA and mRNA, either double stranded or single stranded or used in the form of a polypeptide.

The polynucleotide may be comprised in a vector suitable for transfecting or transducing a host cell. As an RNA, the antigenic unit may be comprised in a vector containing sequences which have shown to increase the stability and translational efficacy of the RNA, e.g. a poly(A) tail. The vector may further comprise a sequence encoding a signal peptide or a fragment thereof. The RNA or vector may be used as an RNA vaccine, which may be formulated as described herein.

Alternatively, the polypeptide may be used as a peptide vaccine which may be formulated as known in the art, i.e. comprising a pharmaceutical carrier and optionally excipients and/or adjuvants known in the art for use in such peptide vaccines.

### DESCRIPTION OF THE DRAWINGS

FIG. 1 shows an immunogenic construct of the invention which is a polypeptide and described as having an N-terminal start and a C-terminal end. The units/elements of the polypeptide—here targeting unit that targets antigen presenting cells (APCs) (TU), dimerization unit (DimU) and antigenic unit—may be arranged in the polypeptide such that the antigenic unit is located at the C-terminal end of the polypeptide (FIG. 1a) or at the N-terminal start of the polypeptide (FIG. 1b). Further details are provided in the section with the title “Immunogenic construct”.

FIG. 2 shows a schematic drawing of a VB10.COV2 construct, an embodiment of the invention as discussed in the Example section of this application.

FIG. 3 shows the T cell response (corrected for PBS negative control) induced with one dose of 25 μg of VB10.COV2 DNA plasmid in C57BL/6-Mcph1Tg(HLA-A2.1)1Enge/J transgenic mice. Total number of IFN-γ positive spots/1×10⁶ pooled splenocytes harvested 14 days post vaccination from mice (n=4) vaccinated with one dose of 25 μg of VB10.COV2 DNA plasmid and stimulated with 2 g/mL peptides/peptide pools composed of peptides corresponding to human HLA-A2.1 epitopes/groups of epitopes present in the respective constructs.

FIG. 4 shows the T cell response (corrected for PBS negative control) induced with one dose of 50 μg of VB10.COV2 DNA plasmid in C57BL/6-Mcph1Tg(HLA-A2.1)1Enge/J transgenic mice. Total number of IFN-γ positive spots/1×10⁶ pooled splenocytes harvested 14 days post vaccination from mice (n=3) vaccinated with one dose of 50 μg of VB10.COV2 DNA plasmid and stimulated with 4 g/mL peptides/peptide pools composed of peptides corresponding to human HLA-A2.1 epitopes/groups of epitopes present in the respective constructs.

FIG. 5 shows the T cell response (corrected for PBS negative control) induced with one dose of 25 μg of VB10.COV2 DNA plasmid in BALB/c mice. Total number of IFN-7 positive spots/1×10⁶ pooled splenocytes harvested 14 days post vaccination from mice (n=3) vaccinated with 25 μg of VB10.COV2 DNA plasmid and stimulated with 2 μg/mL peptide pools composed of overlapping 15-mer peptides covering all the epitopes present in the respective constructs.

FIG. 6 shows the T cell response (corrected for PBS negative control) induced with one dose of 25 μg of VB10.COV2 DNA plasmid in C57BL/6 mice. Total number of IFN-γ positive spots/1×10⁶ pooled splenocytes harvested 14 days post vaccination from mice (n=3) vaccinated with 25 μg of VB10.COV2 DNA plasmid and stimulated with 2 μg/ml peptide pools composed of overlapping 15-mer peptides covering all the epitopes present in the respective constructs.

FIG. 7 shows the T cell response (corrected for PBS negative control) induced with one dose of 25 μg of VB2210 DNA plasmid in C57BL/6 mice. Total number of IFN-γ positive spots/1×10⁶ pooled splenocytes harvested 14 days post vaccination from mice (n=3) vaccinated with 25 μg of VB2210 and stimulated with 4 μg/ml peptides/peptide pools composed of peptides corresponding to human HLA-A2.1 epitopes/groups of epitopes present in the construct.

FIG. 8 shows the T cell response (corrected for PBS negative control) induced with one or two doses of 1, 5 or 25 μg of VB2210 DNA plasmid in C57BL/6-Mcph1Tg(HLA-A2.1)1Enge/J transgenic mice. Total number of IFN-γ positive spots/1×10⁶ splenocytes harvested 14 days post first or post second vaccination (second vaccination at day 21) from mice (n=4) vaccinated with one or two doses of 1, 5 or 25 μg VB2210 DNA plasmid and stimulated with 2 μg/ml peptide pools composed of overlapping 15-mer peptides covering all the epitopes/groups of epitopes contained in the construct. Data are presented as an average of total responses ±SEM.

FIG. 9 shows the T cell response (corrected for PBS negative control) induced with one or two doses of 1, 5 or 25 μg of VB2210 DNA plasmid in C57BL/6-Mcph1Tg(HLA-A2.1)1Enge/J transgenic mice. Total number of IFN-γ positive spots/1×10⁶ splenocytes harvested from mice (n=4) vaccinated with one or two doses of 1, 5 or 25 μg of VB2210 DNA plasmid and stimulated with 4 μg/ml peptide pools composed of peptides covering specific human HLA-A2.1 epitopes contained in the construct. Splenocytes were harvested 14 days post single or post second vaccination (second dose at day 21). Data are presented as an average of total responses ±SEM.

FIG. 10 shows the T cell response induced with one or two doses of 1, 5 or 25 μg of VB2210 DNA plasmid in C57BL/6 wildtype mice. Total number of IFN-γ positive spots/1×10⁶ splenocytes harvested from mice vaccinated with one or two doses of 1, 5 or 25 μg of VB2210 DNA plasmid and stimulated with 4 μg/ml peptide pools composed of peptides covering epitopes contained in the construct. Splenocytes were harvested 84 days post single dose or 85 days post first dose (if 2 doses were administered). Data are presented as an average of total responses ±SEM.

FIG. 11 shows the populations of CD8+ T cells expressing one or two cytokines obtained from C57BL/6-Mcph1Tg(HLA-A2.1)1Enge/J transgenic mice vaccinated with one or two doses of 25 μg VB2210 DNA plasmid. Splenocytes were harvested 14 days after first or second vaccination (second vaccination at day 21) and stimulated with 6 μg/ml of a peptide pool composed of immunogenic peptides identified in the previously carried out ELISpot assays corresponding to multiple HLA-A2.1 epitopes present in the construct. Multicolor staining followed by multiparameter functional analysis was performed to assess expression of IFN-γ, TNF-α, IL-2, IL-4, IL-17, and FoxP3 in stimulated cells.

FIG. 12 shows the T cell response (corrected for negative control) induced with two doses of 3 mg of VB10.2210 DNA plasmid in healthy volunteers. Total number of IFN-7 positive spots/1×10⁶ PBMCs before vaccination (baseline) and after 2 vaccinations (peak) from healthy volunteers (n=11) vaccinated with 2 doses of 3 mg VB10.2210 DNA plasmid and stimulated with peptide pools composed of overlapping 15-mer peptides covering all the epitopes present in VB10.2210 and a selection of minimal peptides are shown. Box plots depict median. Whisker depict down to 25th percentile (−1.5 IQR) and up to the 75th percentile (+1.5IQR). FIG. 12a shows the T cell response towards the spike epitopes and FIG. 12b shows the sum of T cell responses to N, M, ORF1/3/10 and ORF7 epitopes.

FIG. 13 shows the T cell response (corrected for negative control) induced with two doses of 3 mg of VB10.2210 DNA plasmid in healthy volunteers. Total number of IFN-7 positive spots/1×10⁶ PBMCs before vaccination (baseline) and after 2 vaccinations (peak) from healthy volunteers (n=11) vaccinated with 2 doses of 3 mg VB10.2210 DNA plasmid and stimulated with peptide pools composed of overlapping 15-mer peptides covering all the epitopes present in VB10.2210 and a selection of minimal peptides are shown. FIG. 13a shows the T cell response towards the spike epitopes, FIG. 13b shows the T cell response towards the M epitopes, FIG. 13c shows the T cell response towards the N epitopes, FIG. 13d shows the T cell response towards the ORF1/3/10 epitopes and FIG. 13e shows the T cell response towards the ORF7 epitopes.

FIG. 14 shows the T cell response (corrected for negative control) induced after 2 doses of 3 mg of VB10.2210 DNA plasmid in 2 distinct participants. Total number of IFN-γ positive spots/1×10⁶ PBMCs before first vaccination (day 0), after first vaccination (day 21) and after second vaccination (day 35) from healthy volunteers stimulated with peptide pools composed of overlapping 15-mer peptides covering all the epitopes present in VB10.2210 and a selection of minimal peptides are shown (FIG. 14a: participant no. 4; FIG. 14b: participant no. 9).

FIG. 15 shows the phenotype of polyfunctional vaccine-specific T cells in healthy volunteers vaccinated with 2 doses of 3 mg VB10.2210 DNA plasmid. PBMCs before first vaccination (baseline) and after 2 vaccinations (peak) were peptide-stimulated for 16 h prior to intracellular staining for phenotype markers (CD4, CD8) and cytokine production (TNF-α and IFN-γ) and subjected to multiparameter analysis by flow cytometry. FIGS. 15a and 15b show the negative control (PBMCs only in cell medium with corresponding DMSO concentration to peptide pool) at baseline and peak, respectively. FIG. 15c shows the gating of CD8+ T cell population and FIG. 15d shows the TNF-α and IFN-γ expression of the CD8+ T cell population stimulated with non-spike epitopes (M, N and ORFs).

### DETAILED DESCRIPTION

The polynucleotide, polypeptide and the multimeric protein are herein denoted an “immunogenic construct” or just “construct”. An “immunogenic construct” is one that elicits an immune response, when administered to a subject in a form suitable for administration and in an amount effective to elicit the immune response (i.e. an immunologically effective amount).

A “subject” is a human individual. A subject may be a patient, i.e. a human individual suffering from a disease caused by SARS-CoV-2 who is in need of a therapeutic treatment. The terms “subject” and “individual” are used interchangeably herein. The terms “human” and “h” are used interchangeably herein to refer to a human.

A “treatment” is a prophylactic treatment or therapeutic treatment.

A “prophylactic treatment” is a treatment administered to a subject who does not display signs or symptoms of, or displays only early signs or symptoms of, a disease caused by SARS-CoV-2, such that treatment is administered for the purpose of preventing or decreasing the risk of developing the disease. A prophylactic treatment functions as a preventative treatment against a disease caused by SARS-CoV-2, or as a treatment that inhibits or reduces further development or enhancement of the disease. The terms “prophylactic treatment”, “prophylaxis” and “prevention” are used interchangeably herein.

A “therapeutic treatment” is a treatment administered to a subject who has been tested positive to SARS-CoV2 and/or displays symptoms or signs of a disease caused by SARS-CoV-2, in which treatment is administered to the subject for the purpose of diminishing or eliminating those signs or symptoms.

A “nucleotide sequence” is a sequence consisting of nucleotides. The terms “nucleotide sequence” and “nucleic acid sequence” are used interchangeably herein.

The section headings used herein are for organizational purposes only and are not to be construed as limiting the subject matter described.

### Immunogenic Construct

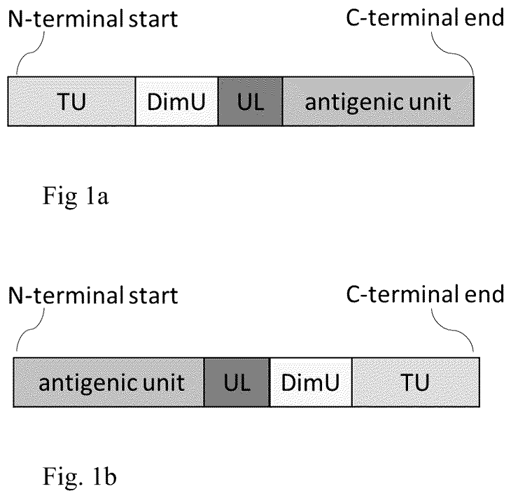

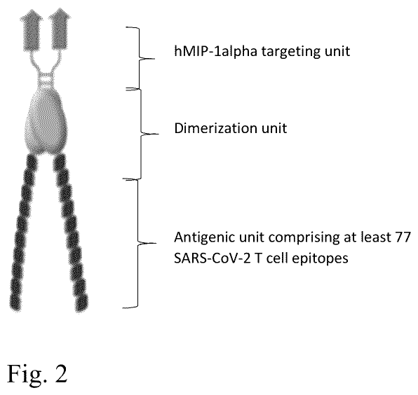

The immunogenic construct of the invention can be described as a polypeptide having an N-terminal start and a C-terminal end (illustrated in FIG. 1). The elements/units of the polypeptide—targeting unit that targets APCs (TU), multimerization unit, such as dimerization unit (DimU) in FIG. 1, and antigenic unit—may be arranged in the polypeptide such that the antigenic unit is located at the C-terminal end of the polypeptide (FIG. 1a) or at the N-terminal start of the polypeptide (FIG. 1b). Preferably, the antigenic unit is located at the C-terminal end of the polypeptide.

The antigenic unit comprises at least 77 SARS-CoV-2 T cell epitopes, either as discrete epitopes or grouped together into one or more groups and may comprise linkers (T cell epitope linkers) which separate the discrete T cell epitopes, the T cell epitopes within a group and/or the groups of T cell epitopes from each other. A unit linker (UL) may connect the multimerization unit and the antigenic unit. The order and orientation of the above-described units and elements is the same in the dimeric protein and the polynucleotide.

In the following, the various units and elements of the construct will be discussed in detail. They are present in the polynucleotide as nucleic acid sequences encoding the units/elements, and in the polypeptide or multimeric protein as amino acids sequences. For the ease of reading, in the following, the units/elements of the construct are mainly explained in relation to the polypeptide/multimeric protein, i.e. on the basis of their amino acid sequences.

### Antigenic Unit

The antigenic unit present in the construct of the invention comprises at least 77 T cell epitopes from SARS-CoV-2. These 77 T cell epitopes are listed in Table 1 below according to the SARS-CoV-2 protein they are derived from, with their respective amino acid sequences and sequence identities (SEQ TD NO):

TABLE 1

| Epitope | | |
|---|---|---|
| number | | |
| and | | SARS- |
| SEQ ID | | CoV-2 |
| NO | Sequence | protein |
| 1 | SRTLSYYKLGASQRVAGDS | M |
| 2 | PKEITVATSRTLSYYKLGA | M |
| 3 | LRIAGHHLGRCDIKDLPKE | M |
| 4 | GRCDIKDLPKEITVATSRT | M |
| 5 | TALTQHGKEDLKFPRGQGV | N |
| 6 | PNNTASWFTALTQHGKEDL | N |
| 7 | SSPDDQIGYYRRATRRIRG | N |
| 8 | QGVPINTNSSPDDQIGYYR | N |
| 9 | EDLKFPRGQGVPINTNSSP | N |
| 10 | VTPSGTWLTYTGAIKLDDK | N |
| 11 | VTPSGTWLTY | N |
| 12 | TYTGAIKLDDKDPNFKDQV | N |
| 13 | TPSGTWLTY | N |
| 14 | MEVTPSGTWL | N |
| 15 | GMSRIGMEVTPSGTWLTYT | N |
| 16 | GMEVTPSGTWL | N |
| 17 | DQVILLNKHIDAYKTFPPT | N |
| 18 | DDKDPNFKDQVILLNKHID | N |
| 19 | SPRWYFYYL | N |
| 20 | LSPRWYFYY | N |
| 21 | HTTDPSFLGRY | QRF1ab |
| 22 | SNEKQEILGTVSW | QRF1ab |
| 23 | ILGTVSWNL | QRF1ab |
| 24 | YFTSDYYQLY | QRF3a |
| 25 | VLHSYFTSDY | QRF3a |
| 26 | SYFTSDYYQL | QRF3a |
| 27 | YFLQSINFVR | QRF3a |
| 28 | VYFLQSINFV | QRF3a |
| 29 | VYFLQSINF | QRF3a |
| 30 | SINFVRIIMR | QRF3a |
| 31 | QSINFVRII | QRF3a |
| 32 | LQSINFVRII | QRF3a |
| 33 | LQSINFVRI | QRF3a |
| 34 | INFVRIIMR | QRF3a |
| 35 | GLEAPFLYLY | QRF3a |
| 36 | FLYLYALVYF | QRF3a |
| 37 | FLQSINFVRI | QRF3a |
| 38 | FLQSINFVR | QRF3a |
| 39 | YLYALVYFL | QRF3a |
| 40 | STQFAFACPDGVKHVYQLRA | QRF7a |
| 41 | GNSPFHPLADNKFALTCFST | QRF7a |
| 42 | DNKFALTCFSTQFAFACPDG | QRF7a |
| 43 | DGVKHVYQLRARSVSPKLFI | QRF7a |
| 44 | TLATCELYHY | QRF7a |
| 45 | LITLATCELY | QRF7a |
| 46 | ITLATCELY | QRF7a |
| 47 | CELYHYQECV | QRF7a |
| 48 | YLCFLAFLL | QRF7b |
| 49 | SLIDFYLCFL | QRF7b |
| 50 | MIELSLIDFY | QRF7b |
| 51 | LLFLVLIML | QRF7b |
| 52 | LIDFYLCFL | QRF7b |
| 53 | IELSLIDFYL | QRF7b |
| 54 | IDFYLCFLAF | QRF7b |
| 55 | FYLCFLAFLL | QRF7b |
| 56 | FYLCFLAFL | QRF7b |
| 57 | FLAFLLFLV | QRF7b |
| 58 | AFLLFLVLI | QRF7b |
| 59 | YINVFAFPF | QRF10 |
| 60 | NVFAFPFTIY | QRF10 |
| 61 | NVFAFPFTI | QRF10 |
| 62 | MGYINVFAF | QRF10 |
| 63 | INVFAFPFTI | QRF10 |
| 64 | GYINVFAFPF | QRF10 |
| 65 | AFPFTIYSL | QRF10 |
| 66 | VLPFNDGVYF | S |
| 67 | VLPFNDGVY | S |
| 68 | LPFNDGVYFA | S |
| 69 | LPFNDGVYF | S |
| 70 | GVYFASTEK | S |
| 71 | DGVYFASTEK | S |
| 72 | QILPDPSKPSKRSFIEDLL | S |
| 73 | PSKRSFIEDLLFNKVTLAD | S |
| 74 | DFGGFNFSQILPDPSKPSK | S |
| 75 | QYGDCLGDIAARDLICAQK | S |
| 76 | LADAGFIKQYGDCLGDIAA | S |
| 77 | DLLFNKVTLADAGFIKQYG | S |

Each of the 77 T cell epitopes comprised in the antigenic unit either has the amino sequence as listed in Table 1 above or an amino acid sequence having at least 73% sequence identity to said listed amino acid sequence. Thus, the antigenic unit comprises the T cell epitope 1, which has the amino acid sequence SEQ ID NO: 1 (i.e. SRTLSYYKLGASQRVAGDS) or an amino acid sequence having at least 73% sequence identity thereto and the T cell epitope 2, which has the amino acid sequence SEQ ID NO: 2 (i.e. PKEITVATSRTLSYYKLGA) or an amino acid sequence having at least 73% sequence identity thereto and the T cell epitope 3, which has the amino acid sequence SEQ ID NO: 3 (i.e. LRIAGHHLGRCDIKDLPKE) or an amino acid sequence having at least 73% sequence identity thereto and so on.

In some embodiments, the 77 SARS-CoV-2 T cell epitopes have amino acid sequences having at least 73% sequence identity to the amino acid sequences SEQ ID NOs: 1-77, such as at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In some other embodiments, the 77 SARS-CoV-2 T cell epitopes have the amino acid sequences of SEQ ID NOs: 1-77.

In some other embodiments, the 77 SARS-CoV-2 T cell epitopes have the amino acid SEQ ID NOs: 1-77, wherein in said sequences 6 or less amino acids are deleted, added or substituted by other amino acids, compared to the amino acid sequences SEQ ID NOs: 1-77, such as 5 or less, 4 or less amino acids, 3 or less amino acids, 2 or less amino acids or 1 or less amino acids. In some embodiments, for T cell epitopes having a length of from 8 to 11 amino acids, 3 or less amino acids, preferably 2 or less amino acids or 1 or less amino acids are deleted, added or substituted by other amino acids, compared to the amino acid sequences SEQ ID NOs: 1-77. In some other embodiments, for T cell epitopes having a length of from 13 to 14 amino acids, 4 or less amino acids, preferably 3 or less amino acids, 2 or less amino acids or 1 or less amino acids are deleted, added or substituted by other amino acids, compared to the amino acid sequences SEQ ID NOs: 1-77. In yet some other embodiments, for T cell epitopes having a length of from 19 to 20 amino acids, 6 or less amino acids, preferably 5 or less, 4 or less amino acids, 3 or less amino acids, 2 or less amino acids or 1 or less amino acids, are deleted, added or substituted by other amino acids, compared to the amino acid sequences SEQ ID NOs: 1-77.

The 77 SARS-CoV-2 T cell epitopes and any additional SARS-CoV-2 T cell epitopes listed in Tables 2 and 3 below which are or may be comprised in the antigenic unit were selected from a pool of T cell epitopes identified in COVID-19 patients, applying the following criteria:
One subset of said T cell epitopes binds to HLA class I alleles, while the other subset binds to HLA class II alleles. Preferably, of the T cell epitopes comprised in the antigenic unit, there is a higher portion that binds to HLA class I alleles than to HLA class II alleles. In one embodiment, at least 60% of the T cell epitopes bind to HLA class I alleles, such as at least 65% or at least 66% or at least 67% or at least 68% or at least 69% or at least 70% or at least 71% or at least 72% or at least 73% or at least 74% or at least 75%
The T cell epitopes are predicted to bind to different HLA class I and class II alleles that cover on average 92% of the world population (ranging from 85% for the West Indies to 99% for Europe), thus a vaccine comprising the construct of the invention should be suitable to be used for treatment and prophylaxis world-wide.
The T cell epitopes are conserved T cell epitopes within a multitude of different SARS CoV-2 strains world-wide. When accounting for the genetic diversity across the SARS-CoV-2 genome, computed using a global collection of samples, the selected epitopes are located in regions with low entropy.

The inclusion of the at least 77 T cell epitopes into the immunogenic construct will ensure that the risk for immune evasion is low compared to spike-only based vaccines, which are at great risk of mutations in key antibody epitopes on the spike surface protein that impairs neutralization.

The T cell epitopes disclosed herein are derived from various SARS-CoV-2 virus structural proteins, i.e. the N (nucleocapsid) protein, the S (spike) protein and the M (membrane) protein and various SARS-CoV-2 virus non-structural proteins, i.e. ORF1ab, ORF3a, ORF7a, ORF7b and ORF10. Within a certain SARS-CoV-2 protein, the T cell epitopes may be derived from the same part of the protein (e.g. the same subunit) or from different parts of said protein. In one embodiment, the T cell epitopes are derived from different parts of a certain SARS-CoV-2 protein. For example, of the 12 T cell epitopes derived from the S protein listed in Table 1, 6 T cell epitopes are derived from the S1 subunit (epitopes 66-71), while 6 T cell epitopes are derived from the S2 subunit (epitopes 72-77).

In the antigenic unit, in some embodiments, some or all of the T cell epitopes disclosed herein are flanked by amino acids sequences also flanking said epitope in the naturally occurring protein the epitope is derived from, e.g. amino acid sequences flanking the epitope in the direction of the N-terminus, the C-terminus or both. Such flanking sequences each may comprise 1 or more, 2 or more, 3 or more, 4 or more, 5 or more, 6 or more, 7 or more, 8 or more, 9 or more or 10 or more amino acids. In a preferred embodiment, such flanking sequences comprise 1 to 10 amino acids, e.g. 2 to 8 amino acids or 3 to 7 amino acids or 4 to 5 amino acids.

In some embodiments, the antigenic unit comprises all of the at least 77 T cell epitopes and, optionally, one or more additional T cell epitopes selected from those listed in Table 2 and 3, as single, discrete epitopes, which may be separated from each other by T cell epitope linkers.

In some other embodiments, the antigenic unit comprises one or more groups comprising at least 2 of the T cell epitopes disclosed herein, e.g. from 2 to 20 epitopes, preferably from 2 to 15 epitopes, such as 2, 3, 4, 5, 6, 7, 8, 8, 10, 11, 12, 13, 14, or 15 epitopes. Within a group, the epitopes may be separated from each other by T cell epitope linkers. Alternatively, the groups of epitopes are separated from each other by T cell epitope linker.

Preferably, the at least 2 T cell epitopes are derived from the same SARS-CoV-2 protein and more preferably from the same part of the same SARS-CoV-2 protein. For example, the antigenic unit may comprise a first group comprising the 4 T cell epitopes derived from the M protein listed in Table 1 (epitopes 1-4), a second group comprising the 6 T cell epitopes which are derived from the S1 subunit (epitopes 66-71) and a third group comprising the 6 T cell epitopes that are derived from the S2 subunit (epitopes 72-77). The epitopes of the first, second and third group may be separated from each other by T cell epitope linkers and the first, second and third group may be separated from each other by T cell epitope linkers. In another embodiment, the third group may be split into two groups: a third group comprising 3 T cell epitopes (72-74) and a fourth group comprising 3 T cell epitopes (75-77).

In some embodiments, the T cell epitopes within a group are sequentially arranged. As an example, the epitopes 1-4 derived from the M protein may be arranged in the order epitope 1-epitope 2-epitope 3-epitope 4 in the first group (or any other combination or permutation of said 4 epitopes). The epitopes may be separated from each other by T cell epitope linkers.

Preferably, the T cell epitopes within a group are aligned to form a continuous sequence of amino acids which corresponds to that of the naturally occurring protein the epitopes are derived from, whereby overlapping sequences are only included once in the continuous sequence and thus in the antigenic unit. As an example, the epitopes 1-4 from the M protein are aligned and the resulting aligned sequence of epitopes (1-4) is included as a group in the antigenic unit:

Epitope 3
LRIAGHHLGRCDIKDLPKE
Epitope 4
GRCDIKDLPKEITVATSRT
Epitope 2
PKEITVATSRTLSYYKLGA
Epitope 1
SRTLSYYKLGASQRVAGDS
Resulting sequence of the group:
LRIAGHHLGRCDIKDLPKEITVATSRTLSYYKLGASQRVAGDS

Also such above-mentioned groups may be flanked by amino acids sequences also flanking the sequence of the group in the naturally occurring protein the sequence of the group is derived from, e.g. amino acid sequences flanking the sequence of the group in the direction of the N-terminus, the C-terminus or both. Such flanking sequences each may comprise 1 or more, 2 or more, 3 or more, 4 or more, 5 or more, 6 or more, 7 or more, 8 or more, 9 or more or 10 or more amino acids. In a preferred embodiment, such flanking sequences comprise 1 to 10 amino acids, e.g. 2 to 8 amino acids or 3 to 7 amino acids or 4 to 5 amino acids. As an example, the above-mentioned sequence of the group of epitopes 1-4 derived from the M protein may comprise the following flanking amino acid (underlined):

HLRIAGHHLGRCDIKDLPKEITVATSRTLSYYKLGASQRVAGDSG

The resulting length of such a group comprising the aligned sequence of at least 2 of the T cell epitopes disclosed herein and optionally flanking amino acid sequences is from about 18 amino acids to about 80 amino acids, e.g. about 20 amino acids to about 70 amino acids or about 21 amino acids to about 67 amino acids.

In yet some other embodiments, the antigenic unit comprises the T cell epitopes disclosed herein in the form of one or more discrete T cell epitopes (e.g. epitope 21, 85 and/or 119) and one or more groups comprising at least 2 T cell epitopes, preferably at least 2 T cell epitopes from the same SARS-CoV-2 protein, more preferably at least 2 T cell epitopes from the same part of the same SARS-CoV-2 protein.

In some embodiments, the following of the at least 77 SARS-CoV-2 T cell epitopes are grouped together: epitopes 1-4; 5-9 (in another embodiment, this group is split into two groups: a first one with epitopes 5-6 and a second one with epitopes 7-9); epitopes 10-18 (in another embodiment, this group is split into two groups: a first one with epitopes 10-16 and a second one with epitopes 17-18); epitopes 19-20; epitopes 22-23; epitopes 24-26; epitopes 27-39; epitopes 40-43; epitopes 44-47; epitopes 48-58; epitopes 59-65; epitopes 66-71 and epitopes 72-77 (in another embodiment, this group is split into two groups: a first one with epitopes 72-74 and a second one with epitopes 75-77).

In some embodiments, the antigenic unit does not comprise T cell epitopes derived from the receptor binding domain (RBD) of the SARS-CoV-2 spike protein. An antigenic unit which only comprises the 77 T cell epitopes listed in Table 1 (or only comprises the 77 T cell epitopes listed in Table 1 and other T cell epitopes which are not derived from the RBD, e.g. one or more of the SARS-CoV-2 T cell epitopes listed in Table 3) would not comprise T cell epitopes derived from RBD.

In some other embodiments, the antigenic unit further comprises SARS-CoV-2 T cell epitopes derived from the RBD, preferably one or more of the 19 SARS-CoV-2 T cell epitopes listed in Table 2 with amino acid sequences having at least 73% sequence identity to the amino acid sequences SEQ ID NOs: 78-96.

TABLE 2

| Epitope | | |
|---|---|---|
| number and | | SARS-CoV-2 |
| SEQ ID NO | Sequence | protein |
| 78 | VIAWNSNNLDSKVGGNYNY | S (RBD) |
| 79 | LPDDFTGCVIAWNSNNLDS | S (RBD) |
| 80 | NYNYLYRLF | S (RBD) |
| 81 | NYLYRLFRK | S (RBD) |
| 82 | LDSKVGGNYNYLYRLFRKS | S (RBD) |
| 83 | ERDISTEIYQAGSTPCNGV | S (RBD) |
| 84 | YNYLYRLFRKSNLKPFERD | S (RBD) |
| 85 | YFPLQSYGF | S (RBD) |
| 86 | VRFPNITNLCPFGEVFNAT | S (RBD) |
| 87 | VRFPNITNL | S (RBD) |
| 88 | VQPTESIVRF | S (RBD) |
| 89 | TESIVRFPNI | S (RBD) |
| 90 | RVQPTESIVRFPNITNLCP | S (RBD) |
| 91 | RFPNITNLCPF | S (RBD) |
| 92 | QPTESIVRF | S (RBD) |
| 93 | FPNITNLCPF | S (RBD) |
| 94 | YAWNRKRISNCVADYSVLY | S (RBD) |
| 95 | SNCVADYSVLYNSASFSTF | S (RBD) |
| 96 | NATRFASVYAWNRKRISNC | S (RBD) |

In some embodiments, the antigenic unit comprises one or more of the 19 SARS-CoV-2 T cell epitopes listed in Table 2, wherein the epitopes have amino acid sequences having at least 73% sequence identity to the amino acid sequences SEQ ID NOs: 78-96, such as at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In another embodiment, the 19 SARS-CoV-2 T cell epitopes listed in Table 2 have the amino acid sequences of SEQ ID NOs: 78-96.

In some other embodiments, the 19 SARS-CoV-2 T cell epitopes listed in Table 2 have the amino acid SEQ ID NOs: 78-96, wherein in said sequences 6 or less amino acids are deleted, added or substituted by other amino acids, compared to the amino acid sequences SEQ ID NOs: 78-96, such as 5 or less, 4 or less amino acids, 3 or less amino acids, 2 or less amino acids or 1 or less amino acids. In one embodiment, for T cell epitopes having a length of from 8 to 11 amino acids, 3 or less amino acids, preferably 2 or less amino acids or 1 or less amino acids are deleted, added or substituted by other amino acids, compared to the amino acid sequences SEQ ID NOs: 78-96. In another embodiment, for T cell epitopes having a length of from 13 to 14 amino acids, 4 or less amino acids, preferably 3 or less amino acids, 2 or less amino acids or 1 or less amino acids are deleted, added or substituted by other amino acids, compared to the amino acid sequences SEQ ID NOs: 78-96. In yet another embodiment, for T cell epitopes having a length of from 19 to 20 amino acids, 6 or less amino acids, preferably 5 or less, 4 or less amino acids, 3 or less amino acids, 2 or less amino acids or 1 or less amino acids, are deleted, added or substituted by other amino acids, compared to the amino acid sequences SEQ ID NOs: 78-96.

In some embodiments, the antigenic unit comprises 2 or more of the 19 SARS-CoV-2 T cell epitopes listed in Table 2. In another embodiment, the antigenic unit comprises 2 or more of the T cell epitopes listed in Table 2 and the following epitopes are preferably grouped together: epitopes 78-84 (in another embodiment, this group is split into two groups: a first one with epitopes 78 and 79 and a second one with epitopes 80-84); epitopes 86-96 (in another embodiment, this group is split in two group: a first one with epitopes 86-93 and a second one with epitopes 94-96).

In yet some other embodiments, the antigenic unit comprises all of the 19 RBD derived SARS-CoV-2 T cell epitopes listed in Table 2. Thus, the antigenic unit of the construct according to the invention comprises at least 96 SARS CoV-2 derived T cell epitopes with amino acid sequences having at least 73% sequence identity to the amino acid sequences SEQ ID NOs: 1-96.

Vaccines comprising immunogenic constructs of the invention whose antigenic units comprise one or more of the RBD derived T cell epitopes listed in Table 2 are preferably administered as booster vaccines to individuals who have previously been vaccinated with vaccines comprising the RBD antigen or parts thereof, preferably vaccines comprising the RBD antigen or parts thereof which are disclosed in PCT/EP2021/061602 and G. Norheim et al., bioRvix 2020, doi: https://doi.org/10.1101/2020.12.08.416875.

In some embodiments, the antigenic further comprises one or more of the 64 SARS-CoV-2 T cell epitopes listed in Table 3 with amino acid sequences having at least 7300 sequence identity to the amino acid sequences SEQ ID NOs: 97-160.

TABLE 3

| Epitope | | |
|---|---|---|
| number | | |
| and | | SARS- |
| SEQ ID | | CoV-2 |
| NO | Sequence | protein |
| 97 | SELVIGAVILRGHLRIAGH | M |
| 98 | SELVIGAVIL | M |
| 99 | SELVIGAVI | M |
| 100 | LNVPLHGTILTRPLLESEL | M |
| 101 | ILTRPLLESELVIGAVILR | M |
| 102 | FNPETNILLNVPLHGTILT | M |
| 103 | WPVTLACFVL | M |
| 104 | WLLWPVTLA | M |
| 105 | TLACFVLAAV | M |
| 106 | LWPVTLACF | M |
| 107 | LWLLWPVTL | M |
| 108 | FLWLLWPVTL | M |
| 109 | FLWLLWPVT | M |
| 110 | NRDVDTDFVNEFY | ORF1ab |
| 111 | DTDFVNEFYAY | ORF1ab |
| 112 | KVPTDNYITTY | ORF1ab |
| 113 | ALRKVPTDNYITTY | ORF1ab |
| 114 | YTEKWESGVKDCVVLHSYF | ORF3a |
| 115 | EHDYQIGGYTEKWESGVKD | ORF3a |
| 116 | VKDCVVLHSYFTSDYYQLY | ORF3a |
| 117 | SYFTSDYYQLYSTQLSTDT | ORF3a |
| 118 | QLYSTQLSTDTGVEHVTFF | ORF3a |
| 119 | ALSKGVHFV | ORF3a |
| 120 | VEHVTFFIY | ORF3a |
| 121 | STDTGVEHVTFFIY | ORF3a |
| 122 | HVTFFIYNK | ORF3a |
| 123 | GVEHVTFFIY | ORF3a |
| 124 | YDANYFLCW | ORF3a |
| 125 | PLLYDANYFL | ORF3a |
| 126 | NPLLYDANY | ORF3a |
| 127 | LYDANYFLCW | ORF3a |
| 128 | LLYDANYFLC | ORF3a |
| 129 | LLYDANYFL | ORF3a |
| 130 | VQELYSPIFL | ORF7a |
| 131 | VQELYSPIF | ORF7a |
| 132 | QELYSPIFL | ORF7a |
| 133 | LYSPIFLIV | ORF7a |
| 134 | ELYSPIFLI | ORF7a |
| 135 | YSKWYIRVGARKSAPLIELC | ORF8 |
| 136 | LCVDEAGSKSPIQYIDIGNY | ORF8 |
| 137 | ARKSAPLIELCVDEAGSKSP | ORF8 |
| 138 | YYVGYLQPRTF | S |
| 139 | YLQPRTFLL | S |
| 140 | YLQPRTFL | S |
| 141 | NLLLQYGSFCTQLNRALTG | S |
| 142 | FCTQLNRALTGIAVEQDKN | S |
| 143 | CGDSTECSNLLLQYGSFCT | S |
| 144 | LTGIAVEQDKNTQEVFAQV | S |
| 145 | DKNTQEVFAQVKQIYKTPP | S |
| 146 | AQVKQIYKTPPIKDFGGFN | S |
| 147 | PLVSSQCVNLTTRTQLPPA | S |
| 148 | NLTTRTQLPPAYTNSFTRG | S |
| 149 | MFVFLVLLPLVSSQCVNLT | S |
| 150 | TRGVYYPDKVFRSSVLHST | S |
| 151 | PPAYTNSFTRGVYYPDKVF | S |
| 152 | KVFRSSVLHSTQDLFLPFF | S |
| 153 | YSSANNCTFEYVSQPFLMD | S |
| 154 | VYSSANNCTF | S |
| 155 | SSANNCTFEY | S |
| 156 | LMDLEGKQGNFKNLREFVF | S |
| 157 | FEYVSQPFLMDLEGKQGNF | S |
| 158 | YFKIYSKHTPINLVRDLPQ | S |
| 159 | GNFKNLREFVFKNIDGYFK | S |
| 160 | FVFKNIDGYFKIYSKHTPI | S |

None of the T cell epitopes listed in Table 3 are derived from RBD.

In some embodiments, the antigenic unit comprises one or more of the 64 SARS-CoV-2 T cell epitopes listed in Table 3, wherein the epitopes have amino acid sequences having at least 73% sequence identity to the amino acid sequences SEQ TD NOs: 97-160, such as at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In another embodiment, the 64 SARS-CoV-2 T cell epitopes listed in Table 3 have the amino acid sequences of SEQ ID NOs: 97-160.

In some other embodiments, the 64 SARS-CoV-2 T cell epitopes listed in Table 2 have the amino acid SEQ ID NOs: 97-160, wherein in said sequences 6 or less amino acids are deleted, added or substituted by other amino acids, compared to the amino acid sequences SEQ ID NOs: 97-160, such as 5 or less, 4 or less amino acids, 3 or less amino acids, 2 or less amino acids or 1 or less amino acids. In one embodiment, for T cell epitopes having a length of from 8 to 11 amino acids, 3 or less amino acids, preferably 2 or less amino acids or 1 or less amino acids are deleted, added or substituted by other amino acids, compared to the amino acid sequences SEQ ID NOs: 97-160. In some other embodiments, for T cell epitopes having a length of from 13 to 14 amino acids, 4 or less amino acids, preferably 3 or less amino acids, 2 or less amino acids or 1 or less amino acids are deleted, added or substituted by other amino acids, compared to the amino acid sequences SEQ ID NOs: 97-160. In yet some other embodiments, for T cell epitopes having a length of from 19 to 20 amino acids, 6 or less amino acids, preferably 5 or less, 4 or less amino acids, 3 or less amino acids, 2 or less amino acids or 1 or less amino acids, are deleted, added or substituted by other amino acids, compared to the amino acid sequences SEQ ID NOs: 97-160.

In some other embodiments, the antigenic unit comprises 2 or more of the T cell epitopes listed in Table 3. In another embodiment, the antigenic unit comprises 2 or more of the T cell epitopes listed in Table 3 and the following epitopes are preferably grouped together: epitopes 97-102; epitopes 103-109; epitopes 110-111; epitopes 112-113; epitopes 114-118 (in some other embodiments, this group is split in two group: a first one with epitopes 114-115 and a second one with epitopes 116-118); epitopes 120-123; epitopes 124-129; epitopes 130-134; epitopes 135-137; epitopes 138-140; epitopes 141-146 (in some other embodiments, this group is split in two group: a first one with epitopes 141-143 and a second one with epitopes 144-146); epitopes 147-152 (in some other embodiments, this group is split in two group: a first one with epitopes 147-149 and a second one with epitopes 150-152); epitopes 153-160 (in some other embodiments, this group is split in two group: a first one with epitopes 153-157 and a second one with epitopes 158-160).

In yet some other embodiments, epitopes from Table 1 and Table 3 are grouped together, for example in a group comprising the epitopes 24-26 and 114-118.

In yet some other embodiments, the antigenic unit comprises all of the 64 SARS-CoV-2 derived T cell epitopes listed in Table 3. Thus, the antigenic unit of the construct according to the invention comprises at least 141 SARS CoV-2 derived T cell epitopes with amino acid sequences having at least 73% sequence identity to the amino acid sequences SEQ ID NOs: 1-77 and 97-160. In some embodiments, the aforementioned antigenic unit does not comprise T cell epitopes derived from the receptor binding domain (RBD) of the SARS-CoV-2 spike protein. In some other embodiments, the aforementioned antigenic unit comprises in addition one or more of the 19 RBD derived T cell epitopes listed in Table 2.

In yet some other embodiments, the antigenic unit comprises all of the SARS-CoV-2 derived T cell epitopes listed in Table 3 and all of the RBD derived T cell epitopes listed in Table 2. Thus, the antigenic unit of the construct according to the invention comprises at least 160 SARS CoV-2 derived T cell epitopes with amino acid sequences having at least 73% sequence identity to the amino acid sequences SEQ ID NOs: 1-160.

### Further Embodiments of the Antigenic Unit

In some embodiments, the T cell epitopes or groups of T cell epitopes are randomly arranged in the antigenic unit. In some other embodiments, the T cell epitopes are arranged in the antigenic unit in such a way, that the most hydrophobic T cell epitopes are located in the middle or towards the middle of the antigenic unit, while the most hydrophilic T cell epitopes are located at or towards the N-terminal and C-terminal part of the antigenic unit. In yet some other embodiments, the groups of T cell epitopes having the most hydrophobic sequences are located in the middle or towards the middle of the antigenic unit while the groups of T cell epitopes having the most hydrophilic sequences are located at or towards the N-terminal and C-terminal part of the antigenic unit. In some embodiments, the most hydrophobic T cell epitopes or group of T cell epitopes may be positioned substantially in the middle of the antigenic unit and the most hydrophilic T cell epitopes or group of T cell epitopes may be positioned towards the N-terminal and C-terminal part of the antigenic unit.

Since a true positioning in the middle of the antigenic unit is only possible if the unit comprises an odd number of T cell epitopes or groups of T cell epitopes, the term “substantially” in this context refers to antigenic units comprising an even number of T cell epitopes or groups of T cell epitopes, wherein the most hydrophobic T cell epitopes are positioned as close to the middle as possible.

Alternatively, the T cell epitopes or groups of T cell epitopes may be arranged alternating between a hydrophilic and a hydrophobic T cell epitope/group of T cell epitopes. Preferably, GC rich T cell epitopes or groups of T cell epitopes are arranged in such a way, that GC clusters are avoided. In some embodiments, GC rich T cell epitopes or groups of T cell epitopes are arranged such that there is at least one non-GC rich T cell epitope/group of T cell epitopes between them.

### Linkers in the Antigenic Unit

In some embodiments, the T cell epitopes or groups of T cell epitopes are separated from each other by T cell epitope linkers (hereinafter also “linker”). A T cell epitope linker may also be included at the N-terminus or C-terminus of the antigenic unit.

In some other embodiments, the T cell epitope linker is designed to be non-immunogenic. A T cell epitope linker may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other. Both types of linkers are useful.

In some embodiments, the T cell epitope linker is flexible linker, which allows for presenting the T cell epitopes or group of T cell epitopes in an optimal manner to the immune system, even if the antigenic unit comprises a large number of T cell epitopes.

In some embodiments, the T cell epitope linker is a peptide consisting of from 4 to 40 amino acids, e.g. 35, 30, 25 or 20 amino acids, e.g. from 5 to 20 amino acids or 5 to 15 amino acids or 8 to 20 amino acids or 8 to 15 amino acids 10 to 15 amino acids or 8 to 12 amino acids. In some other embodiments, the T cell epitope linker consists of 10 amino acids. Preferably, the T cell epitope linker is a peptide consisting of from 4 to 20 amino acids, e.g. from 5 to 18 amino acids or 6 to 15 amino acids or 7 to 10 amino acids. In a particular preferred embodiment, the T cell epitope linker consists of 5 to 7 amino acids or 8 to 12 amino acids, such as 5, 6, 7, 8, 9 or 10 amino acids.

In one embodiment, all T cell epitope linkers comprised in the antigenic unit are identical. If, however, one or more of the T cell epitopes or groups of T cell epitopes comprise a sequence similar to that of the linker, it may be an advantage to substitute the neighboring T cell epitope linker with a linker of a different sequence. Also, if a T cell epitope/linker junction is predicted to constitute an epitope in itself, then it is preferred to use a T cell epitope linker of a different sequence. In another embodiment, the antigenic unit comprises several different T cell epitope linkers, such as 2, 3, 4 or 5 different T cell epitope linkers.

In some embodiments, the T cell epitope linker is a flexible linker, preferably a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In some embodiments, the flexible linker is a serine (S) and/or glycine (G) rich linker, i.e. a linker comprising several serine and/or several glycine residues.

In the following, m is an integer from 1 to 5, e.g., 1, 2, 3, 4, or 5. In some embodiments, m is 2.

Preferably, the T cell epitope linker is a serine (S) and/or glycine (G) rich linker, i.e. a linker comprising several serine and/or several glycine residues.

Preferred examples are GGGGS (SEQ ID NO: 186), GGGSS (SEQ ID NO: 187), GGGSG (SEQ ID NO: 188), GGSGG (SEQ ID NO: 189), SGSSGS (SEQ ID NO: 190) or multiple variants thereof such as GGGGSGGGGS (SEQ ID NO: 191), (GGGGS)m (SEQ ID NO: 192), (GGGSS)m (SEQ ID NO: 193), (GGSGG)m (SEQ ID NO: 194), (GGGSG)m (SEQ ID NO: 195) or (SGSSGS)m (SEQ ID NO: 196).

In another embodiment, the serine and/or glycine rich linker further comprises at least one leucine (L) residue, such as at least 1 or at least 2 or at least 3 leucine residues, e.g. 1, 2, 3 or 4 leucine residues.

In some embodiments, the T cell epitope linker comprises or consists of LGGGS (SEQ ID NO: 197), GLGGS (SEQ ID NO: 198), GGLGS (SEQ ID NO: 199), GGGLS (SEQ ID NO: 200) or GGGGL (SEQ ID NO: 201). In some other embodiments, the linker comprises or consists of LGGSG (SEQ ID NO: 202), GLGSG (SEQ ID NO: 203), GGLSG (SEQ ID NO: 204), GGGLG (SEQ ID NO: 205) or GGGSL (SEQ ID NO: 206). In yet some other embodiments, the linker comprises or consists of LGGSS (SEQ ID NO: 207), GLGSS (SEQ ID NO: 208) or GGLSS (SEQ ID NO: 209).

In yet some other embodiments, the T cell epitope linker comprises or consists of LGLGS (SEQ ID NO: 210), GLGLS (SEQ ID NO: 211), GLLGS (SEQ ID NO: 212), LGGLS (SEQ ID NO: 213) or GLGGL (SEQ ID NO: 214). In yet some other embodiments, the linker comprises or consists of LGLSG (SEQ ID NO: 215), GLLSG (SEQ ID NO: 216), GGLSL (SEQ ID NO: 217), GGLLG (SEQ ID NO: 218) or GLGSL (SEQ ID NO: 219). In yet some other embodiments, the linker comprises or consists of LGLSS (SEQ ID NO: 220), or GGLLS (SEQ ID NO: 221).

In some other embodiments, the T cell epitope linker is serine-glycine linker that has a length of 10 amino acids and comprises 1 or 2 leucine residues.

In some embodiments, the T cell epitope linker comprises or consists of LGGGSGGGGS (SEQ ID NO: 222), GLGGSGGGGS (SEQ ID NO: 223), GGLGSGGGGS (SEQ ID NO: 224), GGGLSGGGGS (SEQ ID NO: 225) or GGGGLGGGGS (SEQ ID NO: 226). In some other embodiments, the linker comprises or consists of LGGSGGGGSG (SEQ ID NO: 227), GLGSGGGGSG (SEQ ID NO: 228), GGLSGGGGSG (SEQ ID NO: 229), GGGLGGGGSG (SEQ ID NO: 230) or GGGSLGGGSG (SEQ ID NO: 231). In yet some other embodiments, the linker comprises or consists of LGGSSGGGSS (SEQ ID NO: 232), GLGSSGGGSS (SEQ ID NO: 233), GGLSSGGGSS (SEQ ID NO: 234), GGGLSGGGSS (SEQ ID NO: 235) or GGGSLGGGSS (SEQ ID NO: 236).

In a further embodiment, the T cell epitope linker comprises or consists of LGGGSLGGGS (SEQ ID NO: 237), GLGGSGLGGS (SEQ ID NO: 238), GGLGSGGLGS (SEQ ID NO: 239), GGGLSGGGLS (SEQ ID NO: 240) or GGGGLGGGGL (SEQ ID NO: 241). In some other embodiments, the linker comprises or consists of LGGSGLGGSG (SEQ ID NO: 242), GLGSGGLGSG (SEQ ID NO: 243), GGLSGGGLSG (SEQ ID NO: 244), GGGLGGGGLG (SEQ ID NO: 245) or GGGSLGGGSL (SEQ ID NO: 246). In yet some other embodiments, the linker comprises or consists of LGGSSLGGSS (SEQ ID NO: 247), GLGSSGLGSS (SEQ ID NO: 248) or GGLSSGGLSS (SEQ ID NO: 249).

In yet some other embodiments, the T cell epitope linker comprises or consists of GSGGGA (SEQ ID NO: 250), GSGGGAGSGGGA (SEQ ID NO: 251), GSGGGAGSGGGAGSGGGA (SEQ ID NO: 252), GSGGGAGSGGGAGSGGGAGSGGGA (SEQ ID NO: 253) or GENLYFQSGG (SEQ ID NO: 254). In yet some other embodiments, the linker comprises or consists of SGGGSSGGGS (SEQ ID NO: 255), SSGGGSSGGG (SEQ ID NO: 256), GGSGGGGSGG (SEQ ID NO: 257), GSGSGSGSGS (SEQ ID NO: 258), GGGSSGGGSG (SEQ ID NO: 259), GGGSSS (SEQ ID NO: 260), GGGSSGGGSSGGGSS (SEQ ID NO: 261) or GLGGLAAA (SEQ ID NO: 262).

In some other embodiments, the linker is a rigid linker. Such rigid linkers may be useful to efficiently separate (larger) antigens and prevent their interferences with each other. In some embodiments, the linker comprises or consist of KPEPKPAPAPKP (SEQ ID NO: 263), AEAAAKEAAAKA (SEQ ID NO: 264), (EAAAK)m (SEQ ID NO: 265), PSRLEEELRRRLTEP (SEQ ID NO: 266) or SACYCELS (SEQ ID NO: 267). In yet some other embodiments, the linker comprises or consists of TQKSLSLSPGKGLGGL (SEQ ID NO: 268). In yet some other embodiments, the inker comprises or consists of SLSLSPGKGLGGL (SEQ ID NO: 269).

In yet some other embodiments, the linker comprises or consists of GGSAGGSGSGSSGGSSGASGTGTAGGTGSGSGTGSG (SEQ ID NO: 270); or GGSGGGSEGGGSEGGGSEGGGSEGGGSEGGGSGGGS (SEQ ID NO: 271) or ELKTPLGDTTHT (SEQ ID NO: 272) or EPKSCDTPPPCPRCP (SEQ ID NO: 273).

Examples of T cell epitope linkers are disclosed in paragraphs [0098]-[0099] and in the recited sequences of WO 2020/176797A1, which is incorporated herein by reference and in paragraphs [0135] to [0139] of US 2019/0022202A1, which is incorporated herein by reference.

### Design of the Antigenic Unit

The T cell epitopes disclosed herein were identified with the help of advanced bioinformatics workflows. The validation of the immunogenicity of in silico predicted T cell epitopes is normally performed using cellular immune response assays that require live T cells and are limited in sensitivity and throughput. Briefly, for SARS-CoV-2 antigens, co-applicant Adaptive Biotechnologies Corporation used its proprietary MIRA© (Multiplexed Assay for Identification of Receptor Antigen-specificity) (Klinger et al., 2015) and blood samples from individuals diagnosed with COVID-19 to map T cell receptors (TCRs) to more than 500 Class I and Class II peptides derived from all 11 open reading frames (ORFs) of SARS-CoV-2. In parallel, Adaptive also used transgene constructs covering these SARS-CoV-2 ORFs.

These data were combined with data from case-control comparisons generated using Adaptive's immunoSEQ® assay to identify public TCR clonotypes implicated in the SARS-CoV-2 immune response and functionally map TCR clonotypes to SARS-CoV-2 epitopes/HLA (Snyder et al., 2020). 77 of the most immunogenic SARS-CoV-2 T cell epitopes were identified, in addition to 19 of the most immunogenic SARS-CoV-2 RBD derived T cell epitopes, and 64 further highly immunogenic SARS-CoV-2 T cell epitopes.

The conservation of these epitopes was assessed using genetic diversity data from available databases. At the time of this analysis, a total number of 700000 SARS-CoV-2 sequences were available and taken into consideration. In addition, the distribution of all reported single nucleotide polymorphisms (SNPs) that defines each of all reported variants of concern (https://outbreak.info/), were checked across all of the 160 T cell epitopes. Out of the 77 T cell epitopes, which are present in all the immunogenic constructs according to the invention, only 4 and 3 epitopes included some of the SNPs that defines the Delta (B.1.617.2) and Gamma (P.1) variants, respectively.

To assess the coverage of HLA diversity by the chosen T cell epitopes, their binding affinity to the most prevalent 19 HLAs alleles globally (Table 4) were predicted using the MHC class I binding prediction tool NetMHCpan v4.1 (www.services.healthtech.dtu.dk/service.php?NetMHCpan-4.1).

TABLE 4
Global frequencies of most prevalent HLA alleles, source: allele
frequencies net database (www.allelefrequencies.net/).

| HLA alleles | Global frequency |
|---|---|
| HLA-A02:01 | 0.19 |
| HLA-A01:01 | 0.11 |
| HLA-A24:02 | 0.10 |
| HLA-A03:01 | 0.09 |
| HLA-A11:01 | 0.07 |
| HLA-B07:02 | 0.08 |
| HLA-B07:02 | 0.08 |
| HLA-B08:01 | 0.07 |
| HLA-B35:01 | 0.06 |
| HLA-B44:02 | 0.05 |
| HLA-B51:01 | 0.05 |
| HLA-B15:01 | 0.04 |
| HLA-C04:01 | 0.13 |
| HLA-C07:02 | 0.12 |
| HLA-C07:01 | 0.12 |
| HLA-C06:02 | 0.08 |
| HLA-C03:01 | 0.07 |
| HLA-C05:01 | 0.06 |
| HLA-C02:02 | 0.04 |
| HLA-C12:03 | 0.04 |

To further investigate how the chosen T cell epitopes will cover a global population, the population coverage tool of the Epitope Immune Database (www.tools.iedb.org/population/) was used. Based on the analysis, the included set of 96 epitopes will ensure an average coverage of 92.2% of the global population (Table 5).

TABLE 5

| | Projected population |
|---|---|
| Geographic area | coverage |
| Central Africa | 92.61% |
| East Africa | 88.88% |
| East Asia | 93.42% |
| Europe | 99.63% |
| North Africa | 95.35% |
| North America | 97.66% |
| Northeast Asia | 90.57% |
| Oceania | 95.27% |
| South Africa | 88.18% |
| South America | 86.58% |
| South Asia | 91.37% |
| Southeast Asia | 90.52% |
| Southwest Asia | 92.0% |
| West Africa | 90.79% |
| West Indies | 85.43% |
| Average | 92.28% |

In addition to assessing population coverage through predicted HLA epitope binding approaches, hypothesized HLA restrictions for the identified immune-dominant epitopes were determined using meta-analysis of Adaptive's MIRA data (Snyder et al., 2020).

More specifically, for each MIRA experiment run, the number of unique T cell lineages responding to epitopes across the SARS-CoV-2 genome were defined in hundreds of assayed individuals. In addition to these T cell readouts, HLA typing of donor material was performed to 4 digit precision, which allowed for the assessment of putative HLA-restrictions of the measured T cell response by searching for alleles correlated with increased yields of antigen specific TCRs. This analysis was performed using a one tailed Wilcox test for each HLA allele present in 3 or more donors and further enriched for HLA specificity by requiring each association to exhibit 2-fold enrichment in their median TCR response between HLA positive versus negative donor sets. No correction was made for HLA linkage disequilibrium and only HLA associations surviving Bonferroni correction were reported. In total, 44 HLA class I and 14 HLA class II restricted epitopes were identified using this approach.

A case-control immune sequencing cohort of several thousand individuals was used to identify shared, public T cell receptors associated with SARS-CoV-2 infection. A set of “enhanced sequences” was defined which represented the TCRb sequences enriched in cases and not in controls using Fisher's exact test. These enhanced sequences should comprise receptors that are both highly public and likely to be SARS-CoV-2 specific and have been validated together as a tool for monitoring recent or past infection (Dalai et al., 2021). As a further prioritization for candidate epitopes of interest, an overlap analysis between these enhanced sequences and the MIRA data was performed to pinpoint the antigen specificity of these SARS-CoV-2 associated receptors. The epitopes with the largest number of overlapping enhanced sequences were prioritized for inclusion in the immunogenic constructs and compositions of the invention because of their ability to produce highly public T cell responses, which correspond to HLA prevalence. The immunogenic construct and compositions according to the invention therefore comprise a unique and large set of SARS-CoV-2 T cell epitopes which have been validated to induce strong T cell responses in humans.

Several T cell vaccine candidates have been developed and tested in various mouse models. These were able to induce specific cellular immunity to the SARS-CoV-2 T cell epitopes in humanized transgenic mice HLA-DR mice (Meyers et al., 2021), HLA-A2.1 mice (Gauttier et al., 2020) and CD34 mice (Somogyi et al., 2020) and in BALB/c mice (Gritstone COVID-19 Vaccine Technical Information, 2021). Further, several of these T cell vaccine candidates have progressed to clinical development or are close to trial initiation. Compared to these, the immunogenic constructs and vaccines according to the invention have a unique and larger set of SARS-CoV-2 T cell epitopes which also have been previously validated to induce strong T cell responses in humans.

Preferred antigenic units are those having an amino acid sequence having at least 73% sequence identity with SEQ ID NOs 161-167.

Thus, in one embodiment, the antigenic unit comprises an amino acid sequence having at least 73% sequence identity to an amino acid sequence selected from the group consisting of SEQ ID NOs: 161, 162, 163, 164, 165, 166 and 167.

In a preferred embodiment, the antigenic unit comprises an amino acid sequence having at least 73% sequence identity to an amino acid sequence selected from the group consisting of SEQ ID NOs: 161, 162, 163, 164, 165, 166 and 167, such as at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%.

In another embodiment, the 77 SARS-CoV-2 T cell epitopes have the amino acid sequences of SEQ ID NOs: 1-77.

In a more preferred embodiment, the antigenic unit comprises an amino acid sequence selected from the group consisting of SEQ ID NOs: 161, 162, 163, 164, 165, 166 and 167.

In another more preferred embodiment, the antigenic unit consists of an amino acid sequence selected from the group consisting of SEQ ID NOs: 161, 162, 163, 164, 165, 166 and 167.

### Targeting Unit

The construct of the invention comprises a targeting unit that targets antigen presenting cells (APCs), which include dendritic cells (DCs) and subsets thereof.

The term “targeting unit” as used herein refers to a unit that delivers the construct as disclosed herein to an antigen-presenting cell for MHC class II-restricted presentation to CD4+ T cells or for providing cross presentation to CD8+ T cells by MHC class I restriction.

Due to the targeting unit, the construct disclosed herein attracts DCs, neutrophils and other immune cells. Thus, the construct will not only target the antigenic unit comprised therein to specific cells, but in addition facilitate a response-amplifying effect (adjuvant effect) by recruiting specific immune cells to the administration site of a vaccine comprising the construct. This unique mechanism is of great importance in a clinical setting, since the construct can be administered to a subject in the form of a vaccine, which does not need to comprise any adjuvants, since the construct comprised in the vaccine provides the adjuvant effect.

The targeting unit is designed to target the construct of the invention to surface molecules expressed on the APCs, such as molecules expressed exclusively on subsets of DCs.

Examples of such surface molecules on APCs are HLA, cluster of differentiation 14 (CD14), cluster of differentiation 40 (CD40), CLEC9A, chemokine receptors and Toll-like receptors (TLRs). Chemokine receptors include C-C motif chemokine receptor 1 (CCR1), C-C motif chemokine receptor 3 (CCR3), C-C motif chemokine receptor 4 (CCR4), C-C motif chemokine receptor 5 (CCR5), C-C motif chemokine receptor 6 (CCR6), C-C motif chemokine receptor 7 (CCR7), C-C motif chemokine receptor 8 (CCR8) and XCR1. Toll-like receptors include TLR-2, TLR-4 and TLR-5. In one embodiment, the targeting unit is or comprises a moiety that interacts with these surface molecules.

Thus, in some embodiments, the targeting unit comprises or consists of an antibody-binding region, such as the antibody variable domains (VL and VH), with specificity for MHC/HLA, CD14, CD40, CLEC9A or Toll-like receptors, preferably with specificity for human (h) CD14, hCD40, hCLEC9A or human Toll-like receptors. In some other embodiments, the targeting unit comprises or consists of a synthetic or natural ligand. Examples include soluble CD40 ligand (CD40L), preferably hCD40L, natural ligands like chemokines, preferably such as in their human forms, e.g. chemokine ligand 5, also called C-C motif ligand 5 (CCL5 or RANTES), preferably hCCL5, such as hCCL5 with SEQ ID NO: 43, macrophage inflammatory protein alpha and its isoforms, including mouse CCL3 (or MIP-1α), and human isoforms hCCL3, hCCL3L1, hCCL3L2 and hCCL3L3, chemokine ligand 4 (CCL4) and its isoform CCL4L, preferably hCCL4 and hCCL4L, chemokine ligand 19 (CCL19), preferably hCCL19, chemokine ligand 20 (CCL20), preferably hCCL20, chemokine ligand 21 (CCL21), preferably hCCL21, chemokine motif ligand 1 or 2 (XCL1 or XCL2), preferably hXCL1 or hXCL2, and bacterial antigens like for example flagellin.

In some embodiments, the targeting unit has affinity for an MHC class II protein. Thus, in some embodiments, the targeting unit comprises or consists of an antibody-binding region, such as the antibody variable domains (VL and VH), with specificity for MHC class II proteins selected from the group consisting of anti-HLA-DP, anti-HLA-DR and anti-pan HLA class II.

In some other embodiments, the targeting unit has affinity for a surface molecule selected from the group consisting of CD14, CD40, TLR-2, TLR-4 and TLR-5, preferably affinity for a surface molecule selected from the group consisting of hCD14, hCD40, hTLR-2, hTLR-4 and hTLR-5. Thus, in some embodiments, the targeting unit comprises or consist of an antibody-binding region, such as the antibody variable domains (VL and VH), with specificity for CD14, CD40, TLR-2, TLR-4 or TLR-5, such as anti-CD14, anti-CD40, anti-TLR-2, anti-TLR-4 or anti-TLR-5, preferably with specificity for hCD14, hCD40, hTLR-2, hTLR-4 or hTLR-5, such as anti-hCD14, anti-hCD40, anti-hTLR-2, anti-hTLR-4 or anti-hTLR-5.

In yet some other embodiments, the targeting unit comprises or consists of flagellin, which has affinity for TLR-5, such as hTLR-5. In yet some other embodiments, the targeting unit comprises or consists of an antibody-binding region with specificity for CLEC9A, such as anti-CLEC9A or variants thereof, such as anti-CLEC9A Fv or the targeting unit comprises or consists of a CLEC9 ligand, e.g. a CLEC9 ligand comprising or consisting of the nucleic acid sequence with SEQ ID NO: 274 or an amino acid sequence encoded by said nucleic acid sequence. In a preferred embodiment, the targeting unit comprises or consists of an antibody-binding region with specificity for hCLEC9A, such as anti-hCLEC9A or variants thereof, such as anti-hCLEC9A Fv or the targeting unit comprises or consists of a human CLEC9 ligand.

Preferably, the targeting unit has affinity for a chemokine receptor selected from CCR1, CCR3, CCR5 and CCR7, more preferably for a chemokine receptor selected from CCR1, CCR3 and CCR5. In a further preferred embodiment, the targeting unit has affinity for a chemokine receptor selected from hCCR1, hCCR3, hCCR5 and hCCR7, more preferably for a chemokine receptor selected from hCCR1, hCCR3 and hCCR5.

In some embodiments, the targeting unit has affinity for the chemokine receptor CCR7, preferably for the human chemokine receptor CCR7. In some other embodiments, the targeting unit comprises or consists of CCL19, such as CCL19 comprising or consisting of a nucleotide sequence of SEQ ID NO: 275 or an amino acid sequence encoded by said nucleotide sequence, or CCL21, such as the human forms of CCL19 or CCL21.

Preferably, the targeting comprises or consists of chemokine human macrophage inflammatory protein alpha (human MIP-1α (hMIP-1α) variant, also called LD780 or CCL3L1), which binds to its cognate receptors, including CCR1, CCR3 and CCR5, expressed on the cell surface of APCs. The binding of the targeting unit to its cognate receptors leads to internalization of the multimeric protein into the APC and degradation of the protein into small peptides that are loaded onto MHC molecules and presented to CD4+ and CD8+ T cells to induce specific immune responses. Once stimulated, and with help from activated CD4+ T cells, CD8+ T cells will target and kill cells expressing the same antigens, e.g. cancer cells expression such same antigens In one preferred embodiment, the targeting unit comprises an amino acid sequence having at least 80% sequence identity to the amino acid sequence 24-93 of SEQ ID NO: 168.

In a further preferred embodiment, the targeting unit comprises an amino acid sequence having at least 85% sequence identity to the amino acid sequence 24-93 of SEQ ID NO: 168, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence 26 to 93 of SEQ ID NO: 168 or comprises the amino acid sequence 24-93 of SEQ ID NO: 168.

In a more preferred embodiment, the targeting unit consists of an amino acid sequence having at least 80% sequence identity to the amino acid sequence 24-93 of SEQ ID NO: 168.

In a further preferred embodiment, the targeting unit consists of an amino acid sequence having at least 85% sequence identity to the amino acid sequence 24-93 of SEQ ID NO: 168, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the amino acid sequence 26 to 93 of SEQ ID NO: 168 or has the amino acid sequence 24-93 of SEQ ID NO: 168.

In another embodiment, the targeting unit comprises or is anti-pan HLA class II.

In one preferred embodiment, the targeting unit comprises an amino acid sequence having at least 80% sequence identity to the amino acid sequence 20-260 of SEQ ID NO: 169.

In a further preferred embodiment, the targeting unit comprises an amino acid sequence having at least 85% sequence identity to the amino acid sequence 20-260 of SEQ ID NO: 169, such as at least 86% or at least 87%, 88%, 89%, 90%, 91%, 92%, 93%, 94%, 95%, 96%, 97%, 98% or at least 99% sequence identity. In yet a further preferred embodiment, the targeting unit has the amino acid sequence 20-260 of SEQ ID NO: 169.

In a more preferred embodiment, the targeting unit consists of an amino acid sequence having at least 80% sequence identity to the amino acid sequence 20-260 of SEQ ID NO: 169.

In a further preferred embodiment, the targeting unit consists of an amino acid sequence having at least 85% sequence identity to the amino acid sequence 20-260 of SEQ ID NO: 169, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 20-260 of SEQ ID NO: 169.

### Multimerization Unit/Dimerization Unit

The constructs of the invention comprise a multimerization unit, such as a dimerization unit.

The term “multimerization unit” as used herein refers to a sequence of nucleotides or amino acids between the antigenic unit and the targeting unit which, in addition to connecting the antigenic unit and the targeting unit, facilitates multimerization of/joins multiple polypeptides, such as two, three, four or more polypeptides, into a multimeric protein, such as a dimeric protein, a trimeric protein or a tetrameric protein. Furthermore, the multimerization unit also provides flexibility in the multimeric protein to allow optimal binding of the targeting unit to the surface molecules on the APCs, even if they are located at variable distances. The multimerization unit may be any unit that fulfils one or more of these requirements.

Multimerization unit that facilitates multimerization of/joins more than two polypeptides In some embodiments, the multimerization unit is a trimerization unit, such as a collagen-derived trimerization unit, such as a human collagen-derived trimerization domain, such as human collagen XVIII-derived trimerization domain (see for instance A. Alvarez-Cienfuegos et al., Sci Rep 6, 28643 (2016)) or human collagen XV-derived trimerization domain. Thus, in some embodiments, the multimerization unit is a trimerization unit that comprises or consists of the nucleic acid sequence of SEQ ID NO: 276, or an amino acid sequence encoded by said nucleic acid sequence. In some other embodiments, the trimerization unit is the C-terminal domain of T4 fibritin. Thus, in some embodiments, the multimerization unit is a trimerization unit that comprises or consists of the amino acid sequence of SEQ ID NO: 277. In some embodiments, the trimerization unit further comprises a hinge region as described below.

In some other embodiments, the multimerization unit is a tetramerization unit, such as a domain derived from p53, optionally further comprising a hinge region as described below. Thus, in some embodiments, the multimerization unit is a tetramerization unit that comprises or consists of the nucleic acid sequence of SEQ ID NO: 278, or an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two polypeptides into a dimeric protein. The dimerization unit also provides the flexibility in the dimeric protein to allow optimal binding of the targeting unit to the surface molecules on the APCs, even if they are located at variable distances. The dimerization unit may be any unit that fulfils these requirements.

Accordingly, in some embodiments, the construct of the invention comprises a dimerization unit comprising a hinge region. In some other embodiments, the dimerization unit comprises a hinge region and another domain that facilitates dimerization. In yet some other embodiments, the dimerization unit comprises a hinge region, a dimerization unit linker and another domain that facilitates dimerization, wherein the dimerization unit linker connects the hinge region to the other domain that facilitates dimerization. In some embodiments, the dimerization unit linker is a glycine-serine rich linker, preferably GGGSSGGGSG (SEQ ID NO: 259), i.e. the dimerization unit comprises a glycine-serine rich dimerization unit linker and preferably comprises the dimerization unit linker GGGSSGGGSG.

The term “hinge region” refers to an amino acid sequence comprised in the dimerization unit that contributes to joining two of the polypeptides, i.e. facilitates the formation of a dimeric protein. Moreover, the hinge region functions as a flexible spacer, allowing the two targeting units of the dimeric protein to bind simultaneously to two surface molecules on APCs, even if they are located at variable distances. In the context of a multimerization unit that facilitates multimerization of/joins more than two polypeptides, the term “hinge region” refers to an amino acid sequence comprised in such multimerization unit that contributes to joining more than two polypeptides, e.g. three or four polypeptides and/or functioning as a flexible spacer, allowing the multiple targeting units of the multimeric protein to bind simultaneously to multiple surface molecules on APCs, even if they are located at variable distances.

The hinge region may be Ig derived, such as derived from IgG, e.g. IgG1 or IgG2 or IgG3, such as derived from hIg, such as derived from human IgG, e.g. hIgG1 or hIgG2 or hIgG3. In some embodiments, the hinge region is derived from IgM, such as derived from human IgM. In some embodiments, the hinge region comprises or consists of the nucleotide sequence with SEQ ID NO: 279 or an amino acid sequence encoded by said nucleic acid sequence. The hinge region may contribute to the dimerization through the formation of covalent bond(s), e.g. disulfide bridge(s) between cysteines. Thus, in some embodiments, the hinge region has the ability to form one or more covalent bonds. Preferably, the covalent bond is a disulfide bridge.

In some embodiments, the dimerization unit comprises or consists of a hinge exon h1 and hinge exon h4 (human hinge region 1 and human hinge region 4), preferably hinge exon h1 and hinge exon h4 from IgG3, more preferably having an amino acid sequence having at least 80% sequence identity to the amino acid sequence 94-120 of SEQ ID NO: 168.

In a preferred embodiment, the dimerization unit comprises or consists of a hinge exon h1 and hinge exon h4 with an amino acid sequence having at least 85% sequence identity to the amino acid sequence 94-120 of SEQ ID NO: 168, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a hinge exon h1 and hinge exon h4 with the amino acid sequence 94-120 of SEQ ID NO: 168.

In one preferred embodiment, the dimerization unit comprises or consists of the amino acid sequence 94-120 of SEQ ID NO: 168, except that at the most four amino acids have been substituted, deleted or inserted, such as at the most three amino acids, such as at the most two amino acids or such as at the most one amino acid.

In one preferred embodiment, the dimerization unit comprises or consists of a nucleic acid sequence having at least 80% sequence identity to the nucleic acid sequence of SEQ ID NO: 280.

In a further preferred embodiment, the dimerization unit comprises or consists of a nucleic acid sequence having at least 85% sequence identity to the nucleic acid sequence of SEQ ID NO: 280, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 280.

In some other embodiments, the dimerization unit comprises another domain that facilitates dimerization, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG, such as from human IgG3. More preferably, the other domain that facilitates dimerization is a carboxyterminal C domain derived from IgG3, such as from human IgG3.

In some embodiments, the dimerization unit comprises or consists of a carboxyterminal C domain derived from IgG3 with an amino acid sequence having at least 80% sequence identity to the amino acid sequence 131-237 of SEQ ID NO: 168.

In a preferred embodiment, the dimerization unit comprises or consists of a carboxyterminal C domain derived from IgG3 with an amino acid sequence having at least 85% sequence identity to the amino acid sequence 131-237 of SEQ ID NO: 168, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a carboxyterminal C domain derived from IgG3 with the amino acid sequence 131-237 of SEQ ID NO: 168.

In one preferred embodiment, the dimerization unit comprises or consists of the amino acid sequence 131-237 of SEQ ID NO: 168, except that at the most 16 amino acids have been substituted, deleted or inserted, such as at the most 15, 14, 13, 12, 11, 10, 9, 8, 7, 6, 5, 4, 3, 2, or 1 amino acid.

In one preferred embodiment, the dimerization unit comprises or consists of a nucleic acid sequence having at least 80% sequence identity to the nucleic acid sequence of SEQ ID NO: 281.

In a further preferred embodiment, the dimerization unit comprises or consists of a nucleic acid sequence having at least 85% sequence identity to the nucleic acid sequence of SEQ ID NO: 281, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of the nucleic acid sequence of SEQ ID NO: 281.

The immunoglobulin domain contributes to dimerization through non-covalent interactions, e.g. hydrophobic interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3. In another preferred embodiment, the dimerization unit consists of a polypeptide consisting of hinge exon h1, hinge exon h4, a dimerization unit linker and a CH3 domain of human IgG3.

In some embodiments, the dimerization unit linker is a glycine-serine rich linker, preferably GGGSSGGGSG, i.e. the dimerization unit comprises a glycine-serine rich dimerization unit linker and preferably the dimerization unit linker GGGSSGGGSG.

In some embodiments, the dimerization unit comprises an amino acid sequence having at least 80% sequence identity to the amino acid sequence of 94-237 of SEQ ID NO: 168.

In a preferred embodiment, the dimerization unit comprises an amino acid sequence having at least 85% sequence identity to the amino acid sequence 94-237 of SEQ ID NO: 168, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a more preferred embodiment the dimerization unit consists of an amino acid sequence having at least 80% sequence identity to the amino acid sequence 94-237 of SEQ ID NO: 168, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence 94-237 of SEQ ID NO: 168.

In one preferred embodiment, the dimerization unit comprises or consists of the amino acid sequence 94-237 of SEQ ID NO: 168, except that at the most 28 amino acids have been substituted, deleted or inserted, such as at the most 25, 20, 15, 14, 13, 12, 11, 10, 9, 8, 7, 6, 5, 4, 3, 2 or 1 amino acids.

In one preferred embodiment, the dimerization unit comprises or consists of a nucleic acid sequence having at least 80% sequence identity to the nucleic acid sequence of SEQ ID NO: 282.

In a further preferred embodiment, the dimerization unit comprises or consists of a nucleic acid sequence having at least 85% sequence identity to the nucleic acid sequence of SEQ ID NO: 282, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of the nucleic acid sequence of SEQ ID NO: 282.

### Unit Linker

In some embodiments, constructs disclosed herein comprise a unit linker. In some embodiments, the antigenic unit is connected to the targeting unit or multimerization unit by a unit linker. Thus, in some embodiments, constructs disclosed herein comprise a unit linker that connects the antigenic unit to the targeting unit or the multimerization unit. In some embodiments, the unit linker is a non-immunogenic linker and/or flexible or rigid linker.

The unit linker may comprise a restriction site in order to facilitate the construction of the first nucleic acid sequence. In some embodiments, the unit linker is GLGGL (SEQ ID NO: 214) or GLSGL (SEQ ID NO: 283). In some other embodiments, the unit linker comprises or consists of GGGGS (SEQ ID NO: 186), GGGGSGGGGS (SEQ ID NO: 191), (GGGGS)m (SEQ ID NO: 192), EAAAK (SEQ ID NO: 284), (EAAAK)m (SEQ ID NO: 265), (EAAAK)mGS (SEQ ID NO: 285), (EAAK)mGS (SEQ ID NO: 286), GPSRLEEELRRRLTEPG (SEQ ID NO: 287), AAY or HEYGAEALERAG (SEQ ID NO: 288). m is an integer from 1 to 5, e.g., 1, 2, 3, 4, or 5. In some embodiments, m is 2.

### Signal Peptide

In a preferred embodiment, the construct of the invention is a polynucleotide which comprises a nucleotide sequence which further encodes a signal peptide. The signal peptide is either located at the N-terminal end of the targeting unit or the C-terminal end of the targeting unit, depending on the orientation of the targeting unit in the polypeptide (FIG. 1). The signal peptide is designed to allow secretion of the polypeptide encoded by the nucleic acid comprised in the polynucleotide from cells transfected with said polynucleotide. Preferably, the signal peptide is that which is naturally present at the N-terminus of any of the targeting units described herein (also called the natural leader sequence).

Any suitable signal peptide may be used. Examples of suitable peptides are an Ig VH signal peptide, preferably a human Ig VH signal peptide, a human TPA signal peptide, such as SEQ ID NO: 170 and a human MIP1-α signal peptide.

In a preferred embodiment, the polynucleotide comprises a nucleotide sequence encoding a human MIP1-α signal peptide and preferably comprises a nucleotide sequence encoding a human MIP1-α targeting unit.

In a further preferred embodiment, the polynucleotide comprises a nucleotide sequence encoding a signal peptide that comprises an amino acid sequence having at least 85% sequence identity to the amino acid sequence 1-23 of SEQ ID NO: 168, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, polynucleotide comprises a nucleotide sequence encoding a signal peptide that comprises the amino acid sequence 1-23 of SEQ ID NO: 168.

In a more preferred embodiment, the polynucleotide comprises a nucleotide sequence encoding a signal peptide that consists of an amino acid sequence having at least 80% sequence identity to the amino acid sequence 1-23 of SEQ ID NO: 168, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, the polynucleotide comprises a nucleotide sequence encoding a signal peptide with the amino acid sequence 1-23 of SEQ ID NO: 168.

In another preferred embodiment, the polynucleotide comprises a nucleotide sequence encoding an Ig VH signal peptide and preferably comprises a nucleotide sequence encoding an anti-pan HLA class II targeting unit.

In a further preferred embodiment, polynucleotide comprises a nucleotide sequence encoding a signal peptide that comprises an amino acid sequence having at least 85% sequence identity to the amino acid sequence 1-19 of SEQ ID NO: 169, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, polynucleotide comprises a nucleotide sequence encoding a signal peptide that comprises the amino acid sequence 1-19 of SEQ ID NO: 169.

In a more preferred embodiment, the polynucleotide comprises a nucleotide sequence encoding a signal peptide that consists of an amino acid sequence having at least 80% sequence identity to the amino acid sequence 1-19 of SEQ ID NO: 169, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In another preferred embodiment, the polynucleotide comprises a nucleotide sequence encoding a signal peptide with the amino acid sequence 1-19 of SEQ ID NO: 169.

### Preferred Immunogenic Constructs

Preferred immunogenic constructs are those comprising the following units:

### Targeting Unit:

The targeting unit comprises an amino acid sequence having at least 80% sequence identity to the amino acid sequence 24-93 of SEQ ID NO: 168.

In a further preferred embodiment, the targeting unit comprises an amino acid sequence having at least 85% sequence identity to the amino acid sequence 24-93 of SEQ ID NO: 168, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence 24-93 of SEQ ID NO: 168 or comprises the amino acid sequence 26-93 of SEQ ID NO: 168.

In a more preferred embodiment, the targeting unit consists of an amino acid sequence having at least 80% sequence identity to the amino acid sequence 24-93 of SEQ ID NO: 168.

In a further preferred embodiment, the targeting unit consists of an amino acid sequence having at least 85% sequence identity to the amino acid sequence 24-93 of SEQ ID NO: 168, such as at least 86% or at least 87%, 88%, 89%, 90%, 91%, 92%, 93%, 94%, 95%, 96%, 97%, 98% or at least 99% sequence identity. In yet another preferred embodiment, the targeting unit consists of the amino acid sequence 24-93 of SEQ ID NO: 168 or consists of the amino acid sequence 26-93 of SEQ ID NO: 168.

### Dimerization Unit:

The dimerization unit comprises an amino acid sequence having at least 80% sequence identity to the amino acid sequence of 94-237 of SEQ ID NO: 168.

In a preferred embodiment, the dimerization unit comprises an amino acid sequence having at least 85% sequence identity to the amino acid sequence 94-237 of SEQ ID NO: 168, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity. In an even more preferred embodiment, the dimerization unit comprises the amino acid sequence 94-237 of SEQ ID NO: 168.

In a more preferred embodiment the dimerization unit consists of an amino acid sequence having at least 80% sequence identity to the amino acid sequence 94-237 of SEQ ID NO: 168, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence 94-237 of SEQ ID NO: 168.

### Antigenic Unit

The following embodiments are preferred embodiments of the antigenic unit either as comprised in the immunogenic constructs or on its own (for prophylactic or therapeutic use, in a pharmaceutical composition); i.e. without the targeting unit and the multimerization unit, such as dimerization unit, being present.

The antigenic unit comprises an amino acid sequence having at least 80% sequence identity to a sequence selected from the group consisting of SEQ ID NOs: 161, 162, 163, 164, 165, 166 and 167.

In a preferred embodiment, the antigenic unit comprises an amino acid sequence having at least 85% sequence identity to an amino acid sequence selected from the group consisting of SEQ ID NOs: 161, 162, 163, 164, 165, 166 and 167, such as at least 86% or at least 87%, 88%, 89%, 90%, 91%, 92%, 93%, 94%, 95%, 96%, 97%, 98% or at least 99% sequence identity.

In a more preferred embodiment, the antigenic unit comprises an amino acid sequence selected from the group consisting of SEQ ID NOs: 161, 162, 163, 164, 165, 166 and 167.

In another embodiment, the antigenic unit consists of an amino acid sequence having at least 80% sequence identity to a sequence selected from the group consisting of SEQ ID NOs: 161, 162, 163, 164, 165, 166 and 167.

In a preferred embodiment, the antigenic unit consists of an amino acid sequence having at least 85% sequence identity to an amino acid sequence selected from the group consisting of SEQ ID NOs: 161, 162, 163, 164, 165, 166 and 167, such as at least 86% or at least 87%, 88%, 89%, 90%, 91%, 92%, 93%, 94%, 95%, 96%, 97%, 98% or at least 99% sequence identity.

In a more preferred embodiment, the antigenic unit has an amino acid sequence selected from the group consisting of SEQ ID NOs: 161, 162, 163, 164, 165, 166 and 167.

In a most preferred embodiment, the immunogenic constructs is a polypeptide that has an amino acid sequence selected from the group consisting of SEQ ID NOs: 171, 172, 173, 174, 175, 176 and 177 or a dimeric protein consisting of two such polypeptides. In another most preferred embodiment, the immunogenic construct is a polynucleotide comprising a nucleotide sequence encoding a polypeptide that has an amino acid sequence selected from the group consisting of SEQ ID NOs: 178, 179, 180, 181, 182, 183 and 184.

In a further most preferred embodiment, the immunogenic construct is a polypeptide that has the amino acid sequence of SEQ ID NO: 177 or a dimeric protein consisting of two such polypeptides. In another further most preferred embodiment, the immunogenic construct is a polynucleotide comprising a nucleotide sequence encoding a polypeptide that has the amino acid sequence of SEQ ID NO: 184. In yet another further most preferred embodiment, the immunogenic construct is a polynucleotide comprising the nucleotide sequence of SEQ ID NO: 185.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned.

The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

### Polynucleotides

The construct of the invention may be in the form of the polynucleotide as described herein.

A further aspect of the invention is a polynucleotide comprising a nucleotide sequence encoding a targeting unit that targets antigen presenting cells, a multimerization unit such as dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77.

Yet another further aspect of the invention is a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77

The polynucleotide may be a DNA or an RNA, including genomic DNA, cDNA and mRNA, either double stranded or single stranded. In a preferred embodiment, the polynucleotide is a DNA.

In some embodiments, the polynucleotide is human codon optimized.

### Vectors

In some embodiments, the polynucleotide as described herein is comprises in a vector.

Thus, a further aspect of the disclosure is vector comprising a polynucleotide comprising a nucleotide sequence encoding a targeting unit that targets antigen presenting cells, a multimerization unit such as dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77.

Yet another further aspect of the disclosure is a vector comprising a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77.

The vector may be any molecule which is suitable to deliver foreign nucleic acid sequences, such as DNA or RNA, into a cell (in vitro or in vivo) where they are expressed, i.e. expression vectors.

In some embodiments, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In some other embodiments, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In a preferred embodiment, the vector is a DNA plasmid and the polynucleotide is a DNA.

### Polycistronic Vectors

In some embodiments, the above-described vector is a polycistronic vector that allows the expression of the polypeptide or antigenic unit disclosed herein and, in addition, the expression of one or more immunostimulatory compounds, as separate molecules.

A further aspect of the disclosure is a vector comprising:
(A) a polynucleotide comprising a nucleotide sequence encoding a polypeptide comprising a targeting unit that targets antigen presenting cells, a multimerization unit such as dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; and
(B) one or more nucleic acid sequences encoding one or more immunostimulatory compounds, wherein the vector allows for the co-expression of the polypeptide and the one or more immunostimulatory compounds as separate molecules.

A yet further aspect of the disclosure is a vector comprising:
(A) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; and
(B) one or more nucleic acid sequences encoding one or more immunostimulatory compounds,
wherein the vector allows for the co-expression of the antigenic unit and the one or more immunostimulatory compounds as separate molecules.

Polycistronic vectors comprising a polynucleotide comprising a nucleotide sequence encoding polypeptide that comprises a targeting unit, a multimerization unit such as a dimerization unit and an antigenic unit and one or more nucleic acid sequences encoding one or more immunostimulatory compounds, wherein the vector allows for the co-expression of the polypeptide and the one or more immunostimulatory compounds as separate molecules are disclosed by the applicant in PCT/EP2022/062665, the disclosure of which is hereby incorporated by reference.

The polycistronic vector may be any molecule which is suitable to deliver foreign nucleic acid sequences, such as DNA or RNA, into a cell (in vitro or in vivo) where they are expressed, i.e. expression vectors.

In some embodiments, the polycistronic vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In some other embodiments, the polycistronic vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In preferred embodiments, the vector is a polycistronic DNA plasmid. The polycistronic vector of the disclosure will be illustrated discussing a plasmid, e.g. DNA plasmid (i.e. a polycistronic DNA plasmid), but it is understood that the discussion thereof applies also to other polycistronic vectors, e.g. viral vectors.

Polycistronic plasmids are known in the art, hence, the skilled person is able to design and construct the polycistronic plasmid of the disclosure.

In preferred embodiments, the polycistronic plasmid of the disclosure comprises one or more co-expression elements, i.e. nucleic acid sequences which allow co-expression of the polypeptide/antigenic unit and the one or more immunostimulatory compounds from the plasmid as separate molecules.

In some embodiments of the present disclosure, the polycistronic plasmid comprises a co-expression element, which causes that the polypeptide/antigenic unit and the one or more immunostimulatory compounds are transcribed on a single transcript but independently translated. Hence, the presence of the co-expression element results in a final production of separate translation products.

In some embodiments, such co-expression element is an IRES element (internal ribosome entry site). In other embodiments, such co-expression element is a 2A self-cleaving peptide (2A peptide). Both co-expression elements are known in the art. If more than one immunostimulatory compound is expressed from the polycistronic plasmid of the disclosure, an IRES element and/or 2A peptide needs to be present in plasmid, e.g. upstream of each nucleic acid sequence encoding an immunostimulatory compound.

In other embodiments, the polycistronic plasmid comprises a co-expression element which causes that the polypeptide/antigenic unit and the one or more immunostimulatory compounds are transcribed as separate transcripts, which results in separate transcription products and thus separate proteins.

In some embodiments, such co-expression element is a bidirectional promoter. In other embodiments, such co-expression elements are various promotors, i.e. the polycistronic plasmid comprises a promoter for each of the nucleic acid sequences encoding either the polypeptide or the one or more immunostimulatory compounds. Both co-expression elements are known in the art.

The above-described co-expression elements can be combined in any manner, i.e. the polycistronic plasmid of the disclosure may comprise one or several of such same or different co-expression elements.

### Immunostimulatory Compounds

The polycistronic plasmid of the present disclosure comprises one or more nucleic acid sequences encoding one or more immunostimulatory compounds. In some embodiments of the present disclosure, the immunostimulatory compound is a compound that stimulates APCs and the stimulation results in e.g. attraction, activation, maturation and/or proliferation of APCs.

In a first embodiment, the immunostimulatory compound is one that attracts APCs, preferably one that can interact with the following surface molecules on APCs: CCR1 (C-C motif chemokine receptor 1), CCR3 (C-C motif chemokine receptor 3), CCR4 (C-C motif chemokine receptor 4), CCR5 (C-C motif chemokine receptor 5), CCR6 (C-C motif chemokine receptor 6), CCR7 (C motif chemokine receptor 7), CCR8 (C motif chemokine receptor 8) or XCR1 (X-C motif chemokine receptor 1).

In other embodiments, the immunostimulatory compound is selected from the list consisting of CCL4, CCL5, CCL19, CCL20, CCL21, XCL1 or XCL2.

In a second embodiment, the immunostimulatory compound is one that promotes activation and/or maturation of APCs. In some embodiments, the immunostimulatory compound can interact with the following surface molecules on APCs: a receptor of the TNF receptor superfamily, including CD40 (cluster of differentiation 40), CD137 (4-1BB), CD27, ICOSL (CD275) or RANK.

Such immunostimulatory compounds may be selected from the list consisting of CD40L (CD40 ligand, CD154), CD137L (4-1BBL, 4-1BB ligand), CD70, ICOS (CD278) or RANKL.

In other embodiments, the immunostimulatory compound is a cytokine selected from IL-2, IL-10, IL-12, TNFα and IFNγ. In other embodiments, the immunostimulatory compound can be an immune signaling molecule such as MyD88 and TRIF which activate through TLR receptors.

In other embodiments, the immunostimulatory compound can be a viral infection sensor such as for example RIG-1 and MDA-5.

In other embodiments, the immunostimulatory compound can interact with a pattern recognition receptor on APCs, e.g. a Toll-like receptor, including TLR2, TLR4 or TLR5. Such immunostimulatory compounds may be selected from the list consisting of pathogen-associated molecular patterns (PAMPs), such as flagellin, or protein damage-associated molecular patterns (DAMPs), such as HMGB1, HSPs (heat-shock proteins), Calrecticulin and Annexin A1. PAMPs/DAMPs include those can be included as a nucleic acid sequence into the DNA plasmid of the disclosure and will be expressed as functional proteins that may comprise functional groups introduced by post-translational modifications. The aforementioned molecules in turn activate the following receptors on APCs: RAGE, TLR4, TLR9 and TIM-3 (for HMGB1), FPR (for Annexin A1), SREC1, LOX1 and CD91 (for HSP).

In a third embodiment, the immunostimulatory compound is one that promotes growth and/or expansion of APCs. In some embodiments, the immunostimulatory compound can interact with the following surface molecules on APCs: GM-CSF-receptor (granulocyte-macrophage colony-stimulating factor receptor, CD1116), FLT-3R (fms like tyrosine kinase 3, CD135), IL-15R or IL-4R.

In other embodiments, the immunostimulatory compound is a growth factor, such as GM-CSF (granulocyte-macrophage colony-stimulating factor), FLT-3L, IL-15 or IL-4.

In some embodiments, the polycistronic vector comprises nucleic acid sequences encoding 2, 3, 4, 5, 6, 7 or 8 immunostimulatory compounds. In preferred embodiments, the polycistronic vector comprises nucleic acid sequences encoding 2 to 6 immunostimulatory compounds, i.e. 2 or 3 or 4 or 5 or 6 different immunostimulatory compounds. The immunostimulatory compounds may be the same or different, preferably different.

In preferred embodiments, the different immunostimulatory compounds also affect APCs differently in order to stimulate the immune system on many different levels and by that maximize the therapeutic or prophylactic effect of the construct of the disclosure.

As an example, the polycistronic vector comprises nucleic acids encoding 2 different immunostimulatory compounds, with the first one being an immunostimulatory compound that promotes the growth of DCs (e.g. FLT-3L) and the second one being an immunostimulatory compound that promotes activation of DCs (e.g. CD40L).

### Production of the Vector and Host Cells

The vectors disclosed herein are generally suitable for transfecting a host cell for expression of a polypeptide/antigenic unit as disclosed herein and formation of a multimeric protein consisting of multiple of such polypeptides, such as formation of a dimeric protein consisting of two of such polypeptides, if a polypeptide is expressed.

Thus, a further aspect of the disclosure is a host cell comprising a vector comprising a polynucleotide comprising a nucleotide sequence encoding a targeting unit that targets antigen presenting cells, a multimerization unit, such as dimerization unit, and an antigenic unit, wherein the antigenic unit comprises at least 77 SARS-CoV-2 T cell epitopes with amino acid sequences having at least 73% sequence identity to the amino acid sequences SEQ ID NOs: 1-77.

Thus, yet a further aspect of the disclosure is a host cell comprising a vector comprising a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77.

In some embodiments, the host cell which comprises the vector is a cell of a cell culture, e.g. a bacteria cell, and the polypeptide/antigenic unit encoded by the vector is expressed in vitro. In some other embodiments, the host cell which comprises the vector of the invention is a cell of a subject and the polypeptide/antigenic unit encoded by the vector is expressed in said subject, i.e. in vivo, as a result of the administration of the vector to the subject. Suitable host cells for in vitro transfection include prokaryote cells, yeast cells, insect cells or higher eukaryotic cells. Suitable host cells for in vivo transfection are e.g. human muscle cells.

In some embodiments, the vectors allows for easy exchange of the various units described above, particularly the antigenic unit. In some embodiments, the vector is a pUMVC4a vector or a vector comprising NTC9385R vector backbones. The antigenic unit may be exchanged with an antigenic unit cassette restricted by the SfiI restriction enzyme cassette where the 5′ site is incorporated in the nucleotide sequence encoding a GLGGL (SEQ ID NO: 214) or GLSGL (SEQ ID NO: 283) unit linker and the 3′ site is included after the stop codon in the vector.

Engineering and production methods of the vectors, e.g. expression vectors such as DNA and RNA plasmids or viral vectors, are well known and the skilled person will be able to engineer/produce the vectors disclosed herein using such known methods. Moreover, various commercial manufacturers offer services for vector design and production.

In one aspect, the disclosure relates to a method of producing a vector comprising a polynucleotide comprising a nucleotide sequence encoding a targeting unit that targets antigen presenting cells, a multimerization unit, such as dimerization unit, and an antigenic unit, wherein the antigenic unit comprises at least 77 SARS-CoV-2 T cell epitopes with amino acid sequences having at least 73% sequence identity to the amino acid sequences SEQ ID NOs: 1-77, the method comprising:
a) transfecting cells in vitro with the vector;
b) culturing said cells;
c) optionally, lysing the cells to release the vector from the cells; and
d) isolating and optionally purifying the vector.

In a further aspect, the disclosure relates to a method of producing a vector comprising a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77, the method comprising:
a) transfecting cells in vitro with the vector;
b) culturing said cells;
c) optionally, lysing the cells to release the vector from the cells; and
d) isolating and optionally purifying the vector.

### Polypeptides/Antigenic Unit

The construct of the invention may be in the form of a polypeptide encoded by the nucleotide sequence comprised in the polynucleotide as described herein.

A further aspect of the invention is a polypeptide comprising a targeting unit that targets antigen presenting cells, a multimerization unit, such as dimerization unit, and an antigenic unit, wherein the antigenic unit comprises at least 77 SARS-CoV-2 T cell epitopes with amino acid sequences having at least 73% sequence identity to the amino acid sequences SEQ ID NOs: 1-77.

The polypeptide may be expressed in vitro for production of e.g. a pharmaceutical composition, such as vaccine, comprising such polypeptide. Alternatively, the polypeptide may be expressed in vivo as a result of the administration of the polynucleotide as described herein to a subject.

In a further aspect, the disclosure relates to an antigenic unit wherein the antigenic unit comprises at least 77 SARS-CoV-2 T cell epitopes with amino acid sequences having at least 73% sequence identity to the amino acid sequences SEQ ID NOs: 1-77.

The antigenic unit may be expressed in vitro for production of e.g. a pharmaceutical composition, such as vaccine, comprising such antigenic unit. The antigenic unit may be produced in vitro, e.g. by transfecting or transducing cells with a polynucleotide comprising a nucleotide sequence encoding antigenic unit such that the antigenic unit is expressed, culturing the cells, isolating the antigenic unit from the cells (which may mean isolating it from the cells in the cell medium, if the antigenic unit is secreted from the cells into the medium) and optionally purifying the antigenic unit.

Thus, a further aspect of the invention is a method for preparing an antigenic unit comprising at least 77 SARS-CoV-2 T cell epitopes with amino acid sequences having at least 73% sequence identity to the amino acid sequences SEQ ID NOs: 1-77, the method comprises:
a) transfecting or transducing cells with a polynucleotide comprising a nucleotide sequence encoding the antigenic unit;
b) culturing the cells;
c) isolating the antigenic unit from the cells; and
d) optionally purifying the antigenic unit.

Alternatively, the antigenic unit may be expressed in vivo as a result of the administration of a polynucleotide encoding such antigenic unit as described herein to a subject.

### Multimeric Protein/Dimeric Proteins

A further aspect of the invention is a multimeric protein consisting of multiple polypeptides, each of which comprises a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77.

In some embodiments, the disclosure relates to a dimeric protein consisting of two polypeptides, each of which comprises a targeting unit, a dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77.

The multimeric proteins may be homomultimers or heteromultimers. For example, if the multimeric protein is a dimeric protein, said dimeric protein may be a homodimer, i.e. a dimeric protein formed by two identical polypeptide molecules (which comprise identical units/elements). Alternatively, said dimeric protein may be a heterodimer formed by two different polypeptides, wherein e.g. polypeptide 1 and 2 comprise the same targeting units and dimerization units but each comprise different antigenic units. Heteromultimeric proteins can be produced by co-transfecting cells with 2 different vectors—one that comprises a polynucleotide that encodes a polypeptide 1 and another that comprises a polynucleotide that encodes a polypeptide 2 which is different from polypeptide 1—and isolation of the heteromultimeric protein after the polypeptides are expressed and the heteromultimeric proteins are formed. Heteromultimeric proteins may be of relevance if the number of T cell epitopes for inclusion into the antigenic unit would exceed an upper size limit for the antigenic unit. It is preferred that the multimeric protein is a homomultimeric protein.

The multimeric/dimeric protein may be prepared by expression of the polypeptide in vitro, e.g. by transfecting or transducing cells with a polynucleotide comprising a nucleotide sequence encoding the polypeptide such that the polypeptide is expressed and forms the multimeric protein/dimeric protein, culturing the cells, isolating the multimeric protein/dimeric protein from the cells (which may mean isolating the protein from the cells in the cell medium, if the protein is secreted from the cells into the medium) and optionally purifying the protein.

Thus, a further aspect of the invention is a method for preparing a multimeric protein, consisting of multiple polypeptides; each of which comprises a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77, the method comprises:
a) transfecting or transducing cells with a polynucleotide comprising a nucleotide sequence encoding the polypeptide;
b) culturing the cells;
c) isolating the multimeric protein from the cells; and
d) optionally purifying the multimeric protein.

In some embodiments, the method is for preparing a dimeric protein, consisting of two polypeptides; each of which comprises a targeting unit that targets antigen presenting cells, a dimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77, the method comprises:
a) transfecting or transducing cells with a polynucleotide comprising a nucleotide sequence encoding the polypeptide;
b) culturing the cells;
c) isolating the dimeric protein from the cells; and
d) optionally purifying the dimeric protein.

Isolation of the polypeptide/multimeric protein and the optional purification can be carried out by methods known in the art, including precipitation, differential solubilization and chromatography.

The polynucleotide may be comprised in a plasmid for transfection or a vector for transduction.

The above-described multimeric/dimeric proteins may be used as the active ingredient in a protein vaccine for the prophylactic or therapeutic treatment of diseases caused by SARS-CoV-2.

### Medicament

In some embodiments of the present disclosure, the constructs (i.e. polynucleotides, polypeptides/multimeric proteins), antigenic units and vectors disclosed herein are for use as a medicament.

### Pharmaceutical Compositions and Vaccines

The construct of the invention may be administered to a subject in the form of a pharmaceutical composition comprising the construct, e.g. the form of a polynucleotide, vector or multimeric protein, and pharmaceutically acceptable carrier, e.g. in the form of a vaccine.

Alternatively, the antigenic unit as described herein may be administered to a subject in the form of a pharmaceutical composition comprising the antigenic unit, e.g. the form of a polynucleotide, vector or polypeptide, and pharmaceutically acceptable carrier, e.g. in the form of a vaccine.

A “vaccine” as used herein is a pharmaceutical composition comprising the construct as disclosed herein and pharmaceutically acceptable carrier, which may further comprise excipients which are typical for vaccines.

A further aspect of the invention is a vaccine comprising a pharmaceutically acceptable carrier and
(i) a polynucleotide comprising a nucleotide sequence encoding a targeting unit that targets antigen presenting cells, a multimerization unit, such as dimerization unit, and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein, consisting of multiple polypeptides as defined in (ii), such as a dimeric protein consisting of two polypeptides as defined in (ii).

A further aspect of the invention is a pharmaceutical composition comprising a pharmaceutically acceptable carrier and
(i) a polynucleotide comprising a nucleotide sequence encoding a targeting unit that targets antigen presenting cells, a multimerization unit, such as dimerization unit, and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein, consisting of multiple polypeptides as defined in (ii), such as a dimeric protein consisting of two polypeptides as defined in (ii).

A further aspect of the invention is a vaccine comprising a pharmaceutically acceptable carrier and
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

A further aspect of the invention is a pharmaceutical composition comprising a pharmaceutically acceptable carrier and
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

Suitable pharmaceutically acceptable carriers include, but are not limited to, saline, buffered saline, such as PBS, dextrose, water, glycerol, ethanol, aqueous buffers, such as isotonic aqueous buffers or Tyrode's buffer, and combinations thereof.

In some embodiments, the pharmaceutically acceptable carrier is an aqueous buffer. In some other embodiments, the aqueous buffer is Tyrode's buffer, e.g. Tyrode's buffer comprising 140 mM NaCl, 6 mM KCl, 3 mM CaCl2), 2 mM MgCl2, 10 mM 4-(2-hydroxyethyl)-1-piperazineethanesulfonic acid (Hepes) pH 7.4, and 10 mM glucose.

The pharmaceutical composition or vaccine may further comprise an adjuvant.

In some embodiments, vaccines comprising a multimeric protein or a polypeptide as described herein further comprise pharmaceutically acceptable adjuvants including, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA).

For pharmaceutical compositions/vaccines comprising the polynucleotide or vector, the pharmaceutical compositions/vaccines may comprise molecules that facilitate the transfection/transduction of cells with the polynucleotide or vector, e.g. one or more transfection agents that facilitate the transfection of muscle cells of a subject. Transfection agents for polynucleotides are known in the art and include positively charged molecules that interact with negatively charged molecules like DNA or RNA and form a positively charged transfection agent-DNA or transfection agent-RNA complex. Such complexes can interact with negatively charged cell membranes which enables the uptake of the complexes and thus the delivery of the DNA or RNA into the cell.

If the polynucleotide is RNA, e.g. mRNA, the RNA may be formulated in or with a lipid, nanoparticle, micelle, lipoplex nanoparticle (a particle having a diameter making the particle suitable for systemic, in particular intravenous administration, typically having a diameter of less than 1000 nanometers, which is composed of a combination of different lipids) or liposome, especially for intravenous administration. In one embodiment, the lipoplex nanoparticle or liposome includes one or more lipids that form a multilamellar structure that encapsulates the RNA In another embodiment, the one or more lipids includes at least one cationic lipid and at least one helper lipid. In some embodiments, the one or more lipids includes (R)—N,N,N-trimethyl-2,3-dioleyloxy-1-propanaminium chloride (DOTMA) and 1,2-dioleoyl-sn-glycero-3-phosphoethanol-amine (DOPE). mRNA molecules may also be formulated as naked mRNA molecules in a suitable injection buffer.

In some specific embodiments, the pharmaceutical composition/vaccine comprises a pharmaceutically acceptable amphiphilic block co-polymer comprising blocks of poly(ethylene oxide) and polypropylene oxide.

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the pharmaceutical composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In some other embodiments, the pharmaceutical composition comprises the amphiphilic block co-polymer in an amount of from 2% w/v to 5% w/v, such as about 3% w/v.

The pharmaceutical composition/vaccine may be formulated in any way suitable for administration to a subject, e.g. such as a liquid formulation for injection, e.g. for intradermal or intramuscular injection.

The pharmaceutical composition/vaccine may be administered in any way suitable for administration to a subject, such as administered by intradermal, intramuscular, or subcutaneous injection, or by mucosal or epithelial application, such as intranasal or oral.

In a preferred embodiment, the pharmaceutical composition/vaccine comprises a polynucleotide, e.g. comprised in a vector, and is administered by intramuscular or intradermal injection.

The pharmaceutical composition/vaccine of the invention typically comprises the polynucleotide in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The vaccine of the invention typically comprises the polypeptide/multimeric protein in the range of from 5 μg to 5 mg.

The amount of polynucleotide/polypeptide/dimeric protein may vary depending on whether the pharmaceutical composition/vaccine is administered for prophylactic or therapeutic treatment, the severity of the disease in individuals which are infected, and parameters like the age, weight, gender, medical history and pre-existing conditions.

### Methods for Preparing the Pharmaceutical Composition/Vaccine

Suitable methods for preparing the pharmaceutical composition/vaccine according to the invention are disclosed in WO 2004/076489A1, WO 2011/161244A1, WO 2013/092875A1 and WO 2017/118695A1, which are incorporated herein by reference, and include preparing the polynucleotide, vector, polypeptide or multimeric protein by the methods described therein and mixing them with a pharmaceutically acceptable carrier and optionally further pharmaceutically acceptable excipients, like those described in the previous section herein.

In a preferred embodiment, the polynucleotide, vector, polypeptide or multimeric protein are dissolved in said pharmaceutically acceptable carrier.

### Treatment

The pharmaceutical composition/vaccine of the invention may be used to treat diseases caused by SARS-CoV-2 and such treatment may either be for prophylactic or for therapeutic purpose.

The pharmaceutical composition/vaccine is administered such that it induces an immunoprotective response (for a prophylactic treatment) or an immunotherapeutic response (for a therapeutic treatment) in the individual vaccinated with such vaccine/administered with such pharmaceutical composition. Such response is induced by either a single vaccination/administration or several vaccinations/administrations, e.g. an initial vaccination and one or several booster vaccinations, adequately spaced in time.

In a further aspect, the invention provides a method for treating a subject having a disease caused by SARS-CoV-2 or being in need of prevention of such disease, the method comprising administering to the subject a pharmaceutical composition/vaccine comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii).

In some embodiments, the multimerization unit is a dimerization unit and the multimeric protein is a dimeric protein consisting of two polypeptides.

Further, the disclosure provides a method for treating a subject having a disease caused by SARS-CoV-2 or being in need of prevention of such disease, the method comprising administering to the subject a pharmaceutical composition/vaccine comprising a pharmaceutically acceptable carrier and:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

Also disclosed herein is a pharmaceutical composition/vaccine for use in the prophylactic or therapeutic treatment of a disease caused by SARS-CoV-2, the pharmaceutical composition/vaccine comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii).

In some embodiments, the multimerization unit is a dimerization unit and the multimeric protein is a dimeric protein consisting of two polypeptides.

Further, the disclosure provides a pharmaceutical composition/vaccine for use in the prophylactic or therapeutic treatment of a disease caused by SARS-CoV-2, the pharmaceutical composition/vaccine comprising a pharmaceutically acceptable carrier and:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

Also disclosed herein is the use of
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii) for the manufacture a pharmaceutical composition/vaccine for use in the prophylactic or therapeutic treatment of a disease caused by SARS-CoV-2 in a subject, wherein the pharmaceutical composition/vaccine comprises a pharmaceutically acceptable carrier and wherein said pharmaceutical composition/vaccine is administered to said subject.

In some embodiments, the multimerization unit is a dimerization unit and the multimeric protein is a dimeric protein consisting of two polypeptides.

Also disclosed herein is the use of
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i) for the manufacture a pharmaceutical composition/vaccine for use in the prophylactic or therapeutic treatment of a disease caused by SARS-CoV-2 in a subject, wherein the pharmaceutical composition/vaccine comprises a pharmaceutically acceptable carrier and wherein said pharmaceutical composition/vaccine is administered to said subject.

Also disclosed herein is the use of a pharmaceutical composition/vaccine comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii) for treating a subject having a disease caused by SARS-CoV-2 or being in need of prevention of such disease.

In some embodiments, the multimerization unit is a dimerization unit and the multimeric protein is a dimeric protein consisting of two polypeptides.

Also disclosed herein is the use of a pharmaceutical composition/vaccine comprising a pharmaceutically acceptable carrier and:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i) for treating a subject having a disease caused by SARS-CoV-2 or being in need of prevention of such disease.

Also disclosed herein is a pharmaceutical composition/vaccine comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii) when used in the prophylactic or therapeutic treatment of a disease caused by SARS-CoV-2.

In some embodiments, the multimerization unit is a dimerization unit and the multimeric protein is a dimeric protein consisting of two polypeptides.

Also disclosed herein is a pharmaceutical composition/vaccine comprising a pharmaceutically acceptable carrier and:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i) when used in the prophylactic or therapeutic treatment of a disease caused by SARS-CoV-2.

Also disclosed herein is the use of a pharmaceutical composition/vaccine comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii) for the therapeutic or prophylactic treatment of a disease caused by SARS-CoV-2.

In some embodiments, the multimerization unit is a dimerization unit and the multimeric protein is a dimeric protein consisting of two polypeptides.

Also disclosed herein is the use of a pharmaceutical composition/vaccine comprising a pharmaceutically acceptable carrier and:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i) for the therapeutic or prophylactic treatment of a disease caused by SARS-CoV-2.

Also disclosed herein is a medicament for the treatment or prevention of a disease caused by SARS-CoV-2 in a subject having said disease or being in need of prevention of said disease by administering to the subject the medicament comprising a pharmaceutically acceptable carrier and:
(i) a polynucleotide comprising a nucleotide sequence encoding a targeting unit that targets antigen presenting cells, a multimerization unit and an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i); or
(iii) a multimeric protein consisting of multiple polypeptides as defined in (ii).

In some embodiments, the multimerization unit is a dimerization unit and the multimeric protein is a dimeric protein consisting of two polypeptides.

Also disclosed herein is a medicament for the treatment or prevention of a disease caused by SARS-CoV-2 in a subject having said disease or being in need of prevention of said disease by administering to the subject the medicament comprising a pharmaceutically acceptable carrier and:
(i) a polynucleotide comprising a nucleotide sequence an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

In some embodiments of the above-disclosed methods and uses, the pharmaceutical composition/vaccine/medicament comprises the polynucleotide in a vector.

In the method of treatment/use of the pharmaceutical composition/vaccine/medicament disclosed herein, said pharmaceutical composition/vaccine/medicament is preferably administered in a therapeutically effective or prophylactically effective amount. Such amount may be administered in one administration, i.e. one dose, or in several administrations, i.e. repetitive doses, i.e. in a series of doses, e.g. over the course of several days, weeks or months.

The actual dose to be administered may vary and depend on whether the treatment is a prophylactic or therapeutic treatment, the age, weight, gender, medical history, pre-existing conditions and general condition of the subject, the severity of the disease being treated and the judgment of the health care professionals.

In the method of treatment/use of the pharmaceutical composition/vaccine/medicament disclosed herein, said pharmaceutical composition/vaccine/medicament may be administered in way as described herein, e.g. described in the section “pharmaceutical compositions/vaccines”.

The method of treatment/use of the pharmaceutical composition/vaccine/medicament as disclosed herein can be continued for as long as the clinician overseeing the patient's care deems the method to be effective and the treatment to be needed.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

### Design and Production of DNA Immunogenic Constructs According to the Invention

Seven DNA plasmid constructs (in the following, collectively also called VB10.COV2) were designed, produced and tested. All constructs comprise nucleic acid sequences encoding a human MIP-1α (hMIP-1α) targeting unit, (including its signal peptide), a dimerization unit comprising hinge exons 1 and 4 from IgG3 and the CH3 domain of human IgG3 (signal peptide, targeting unit and dimerization unit having the amino acid sequence of SEQ ID NO: 168) and an antigenic unit which comprises at least the 77 SARS CoV2 T cell epitopes with the amino acid sequences SEQ ID NOs: 1-77 listed in Table 1.

### Details for Each of the Constructs are as Follows:

VB2193 comprises a nucleic acid sequence encoding the antigenic unit of SEQ ID NO: 161, comprising the 160 T cell epitopes with the amino acid sequences SEQ ID NOs: 1-160, wherein the epitopes or groups of epitopes are separated from each other by GGGGSGGGGS T cell epitope linkers. VB2193 comprises a nucleic acid sequence encoding the polypeptide having the amino acid sequence of SEQ ID NO: 178.

VB2194 comprises a nucleic acid sequence encoding the antigenic unit of SEQ ID NO: 162, comprising the 160 T cell epitopes with the amino acid sequences SEQ ID NOs: 1-160, wherein the epitopes or groups of epitopes are separated from each other by GGGGSGGGGS T cell epitope linkers. VB2194 comprises a nucleic acid sequence encoding the polypeptide having the amino acid sequence of SEQ ID NO: 179.

VB2196 comprises a nucleic acid sequence encoding the antigenic unit of SEQ ID NO: 163, comprising the 141 T cell epitopes with the amino acid sequences SEQ ID NOs: 1-77 and 97-160, wherein the epitopes or groups of epitopes are separated from each other by GGGGSGGGGS T cell epitope linkers. VB2196 comprises a nucleic acid sequence encoding the polypeptide having the amino acid sequence of SEQ ID NO: 180.

VB2197 comprises a nucleic acid sequence encoding the antigenic unit of SEQ ID NO: 164, comprising the 96 T cell epitopes with the amino acid sequences SEQ ID NO: 1-96, wherein the epitopes or groups of epitopes are separated from each other by GGGGSGGGGS T cell epitope linkers. VB2197 comprises a nucleic acid sequence encoding the polypeptide having the amino acid sequence of SEQ ID NO: 181.

VB2207 comprises a nucleic acid sequence encoding the antigenic unit of SEQ ID NO: 165, comprising the 141 T cell epitopes with the amino acid sequences SEQ ID NO: 1-77 and 97-160, wherein the epitopes or groups of epitopes are separated from each other by SGSSGS or GGSGG T cell epitope linkers. VB2207 comprises a nucleic acid sequence encoding the polypeptide having the amino acid sequence of SEQ ID NO: 182.

VB2208 comprises a nucleic acid encoding the antigenic unit of SEQ ID NO: 166), comprising the 141 T cell epitopes with the amino acid sequences SEQ ID NOs: 1-77 and 97-160, wherein the epitopes or groups of epitopes are separated by SGSSGS or GGSGG T cell epitope linkers. VB2208 comprises a nucleic acid sequence encoding the polypeptide having the amino acid sequence of SEQ ID NO: 183.

VB2210 comprises a nucleic acid encoding the antigenic unit of SEQ ID NO: 167, comprising the 96 T cell epitopes with the amino acid sequences SEQ ID NOs: 1-196, wherein the epitopes or groups of epitopes are separated by GGSGG and SGSSGS T cell epitope linkers. VB2210 comprises a nucleic acid sequence encoding the polypeptide having the amino acid sequence of SEQ ID NO: 184. VB2210 comprises the nucleic acid sequence of SEQ ID NO: 185.

All constructs were ordered from Genscript (860 Centennial Ave., Piscataway, NJ 08854, USA) and cloned into the expression vector pUMVC4a, which contained the sequence of the aforementioned signal peptide, targeting unit and dimerization unit and a unit linker (GLGGL), which includes a restriction site that allows convenient exchange of the respective antigenic units.

### Example 2

Characterization of VB10.COV2 Proteins Produced Post Transfection of HEK293 Cells with VB10.COV2 DNA Plasmids

### Purpose:

The purpose of the study was to characterize the VB10.COV2 proteins post transient in vitro transfection of mammalian cells with the various VB10.COV2 DNA plasmids. The presence of functional VB10.COV2 proteins in the cell supernatant was verified by an ELISA assay by antibodies binding to the human MIP-1α (targeting unit) and human IgG3 CH3-domain (dimerization unit, as capture antibody).

### Methods/Materials:

Transient transfection of HEK293 cells with the VB10.COV2 DNA plasmid: HEK293 cells were purchased from ATCC and were transiently transfected with VB10.COV2 DNA. Briefly, 2×10⁵ cells/well were plated in 24-well tissue culture plates with 10% FBS growth medium and transfected with 1 μg VB10.COV2 DNA plasmid using Lipofectamine® 2000 reagent under the conditions suggested by the manufacturer (Invitrogen by Life Technologies). The transfected cells were then maintained for up to 6 days at 37° C. with 5% CO2 and the cell supernatant was harvested for characterization of the VB10.COV2 protein.

ELISA with Monoclonal Antibodies Binding to hMIP-1α and hIgG3:

The ELISA was performed to verify the amount of VB10.COV2 protein produced by the HEK293 cells and secreted into the cell supernatant. Briefly, MaxiSorp Nunc-immuno plates were coated with 1 μg/ml of anti-human IgG3 CH3 (MCA878G, BioRad) in 1×PBS with 100 μl/well and plates were incubated overnight at 4° C. The microtiter wells were blocked by the addition of 200 μl/well 4% BSA in 1×PBS. 100 μl of cell supernatant from transfected HEK293 cells containing VB10.COV2 proteins were added to the plates. For detection antibody, anti-human MIP-1α biotinylated was added and incubated. Then, strep-HRP (1:3000) was added and incubated. Unless specified, all incubations were carried out at 37° C. for 1 h, followed by 3× washing with PBS-Tween. Afterwards, 100 μl/well of TMB solution was added and color development was stopped after 5-15 min adding 100 μl/well of 1 M HCl. The optical density at 450 nm was determined on an automated plate reader (Thermo Scientific Multiscan GO).

### Results:

Successful expression and secretion of functional VB10.COV2 proteins were observed for all constructs. Conformational correctness of all VB10.COV2 proteins was confirmed by binding to antibodies detecting the hMIP-1α and the CH3 domain of human IgG3 in ELISA.

### Example 3

Induction of IFN-γ T Cells Responses Specific to T Cell Epitopes Comprised in VB10.COV2 DNA Plasmids by Vaccination with Such Constructs

### Purpose:

The purpose of the study was to evaluate the cellular immune response (T cell response) against the T cell epitopes comprised in VB10.COV2 DNA in mice vaccinated with such constructs.

### Methods/Materials:

### General Study Design:

Female 16-week-old BALB/c and 10-week-old C57BL/6 mice were obtained from Janvier Labs (France), and 6-9-week-old C57BL/6-Mcph1Tg(HLA-A2.1)1Enge/J mice were obtained from Jackson Laboratory (USA). The latter are homozygous mice carrying the Tg(HLA-A2.1)1Enge transgene. These mice express significant quantities of the human HLA-A2.1 class I molecules on cells from the spleen, bone marrow and thymus. This transgenic model was used to specifically evaluate responses to the human HLA-A2.1 T cell epitopes comprised in the VB10.COV2 DNA constructs since the other mouse models, expressing mouse MHC class I and II, will not optimally respond to the human T cell epitopes comprised in the constructs. All animals were housed in the animal facility at the Domus Medica at University of Oslo (Oslo, Norway). All animal protocols were approved by the Norwegian Food Safety Authority (Oslo, Norway). Mice were anesthetized and shaved prior to vaccination. For the studies, the mice received VB10.COV2 at doses of 1, 5, 25 or 50 μg DNA i.m. on day 0 and day 21 for boost regime (see vaccination schedule in Table 6). VB10.COV2 DNA plasmid was administrated to the tibialis anterior muscle by needle injection (as 25 μl solution in sterile PBS in each leg) followed by AgilePulse in vivo electroporation (BTX, US), consisting of 3 sets of pulses with 110-450 V. Mice administered with PBS (vehicle only) were included as negative control group in each experiment. On days 14 or 35, spleens were collected.

TABLE 6
Vaccination schedule

| Dose | | Vaccinated | Termination | Mouse model |
|---|---|---|---|---|
| 1 | μg | Day 0 | Day 14 | C57BL/6-Mcph1Tg(HLA-A2.1)1Enge/J |
| 1 | μg | Day 0 and 21 | Day 35 | C57BL/6-Mcph1Tg(HLA-A2.1)1Enge/J |
| 5 | μg | Day 0 | Day 14 | C57BL/6-Mcph1Tg(HLA-A2.1)1Enge/J |
| 5 | μg | Day 0 and 21 | Day 35 | C57BL/6-Mcph1Tg(HLA-A2.1)1Enge/J |
| 25 | μg | Day 0 | Day 14 | C57BL/6-Mcph1Tg(HLA-A2.1)1Enge/J, |
| | | | | C57BL/6, BALB/c |
| 25 | μg | Day 0 and 21 | Day 35 | C57BL/6-Mcph1Tg(HLA-A2.1)1Enge/J |
| 50 | μg | Day 0 | Day 14 | C57BL/6-Mcph1Tg(HLA-A2.1)1Enge/J |

### IFN-γ ELISpot Assay:

The IFN-γ ELISpot assay was performed on fresh splenocytes from mice vaccinated with VB10.COV2 DNA plasmids. The animals were sacrificed at day 14 and 35, and the spleens were harvested aseptically. The spleens were mashed, cell suspensions were incubated with 1×ACK buffer, washed and re-suspended to a cell concentration of 6×10⁵ cells. Furthermore, the cells were plated in triplicates (6×10⁵ cells/well) and stimulated for 24 h with PBS solutions of peptides or peptide pools as follows: for assays performed on splenocytes from transgenic C57BL/6-Mcph1Tg(HLA-A2.1)1Enge/J mice expressing human HLA2-2.1 class I molecules, the stimulation was carried out with a) individual peptides corresponding to HLA-A2.1 epitopes comprised in the respective constructs, as discrete T cell epitope or as the only HLA-A2.1 epitope in a group of epitopes or b) pools of peptides corresponding to HLA-A2.1 epitopes comprised in groups of epitopes present in the respective constructs, wherein such groups comprise more than one HLA-A2.1 epitope. For assays performed on splenocytes from wildtype BALB/c mice or wildtype C57BL/6 mice, the stimulation was carried out with peptide pools used composed of overlapping 15-mer peptides covering all the epitopes/groups of epitopes present in the respective constructs.

PBS/no peptide stimulation was used as negative control. The stimulated splenocytes were analyzed for IFN-γ responses using the IFN-γ ELISpot Plus kit (Mabtech AB, Sweden). Spot-forming cells were measured in a CTL ELISpot reader, ImmunoSpot 5.0.3 from Cellular Technology.

### Results:

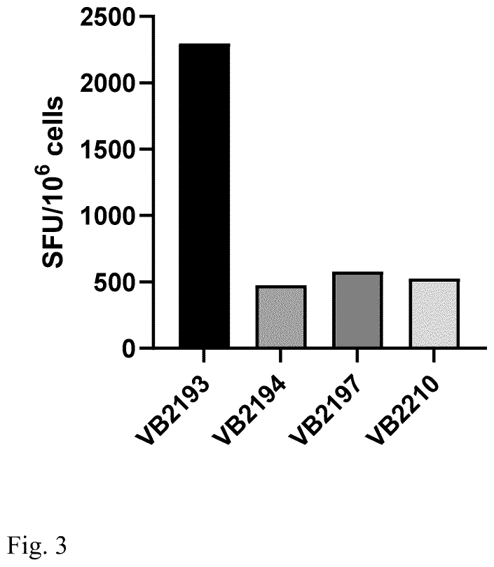

Transgenic C57BL/6-Mcph1Tg(HLA-A2.1)1Enge/J mice expressing human HLA2-2.1 class I molecule were vaccinated with one dose of 25 μg of 4 various VB10.COV2 DNA constructs (VB2193, VB2194, VB2197 and VB2210). In an IFN-γ ELISpot assay 14 days post vaccination, using peptides/pools of peptides as described above, it was demonstrated that the constructs elicited a rapid and strong T cell response in the transgenic mice specifically against the human HLA.A2.1 epitopes present in such constructs (FIG. 3).

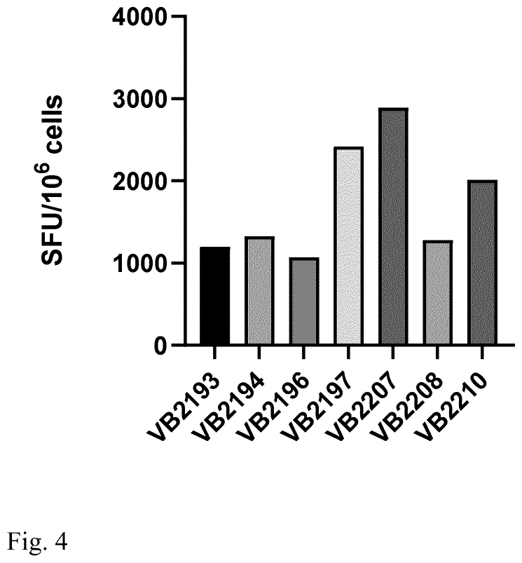

In another experiment, transgenic C57BL/6-Mcph1Tg(HLA-A2.1)1Enge/J mice were vaccinated with one dose of 50 μg of each of the 7 VB10.COV2 constructs, and the IFN-7 ELISpot assay was carried out as described in the previous paragraph. The results showed that all 7 VB10.COV2 constructs induced a rapid and strong T cell responses to the T cell epitopes specific to human HLA-A2.1 molecules in the transgenic mice (FIG. 4), with the response induced by VB2197, VB2207 and VB2210 being about twice as strong than the other 4 constructs.

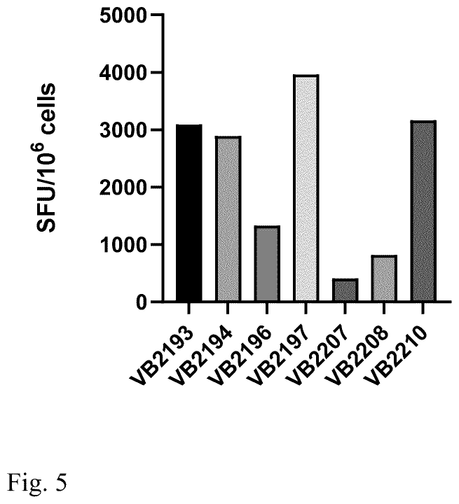

All 7 VB10.COV2 constructs were also tested in the wildtype BALB/c mice which were vaccinated with one dose of 25 μg of each construct. In the ELISpot assay for this experiment 14 days post vaccination, the peptide pools used for stimulation of the splenocytes were composed of overlapping 15-mer peptides covering all the epitopes/groups of epitopes included in the respective constructs. The results showed that all 7 VB10.COV2 constructs were able to induce a rapid and strong T cell responses in the wildtype mice model as well (FIG. 5). VB2193, VB2194, VB2197 and VB2210 induced stronger responses than the other 3 constructs in this mouse model.

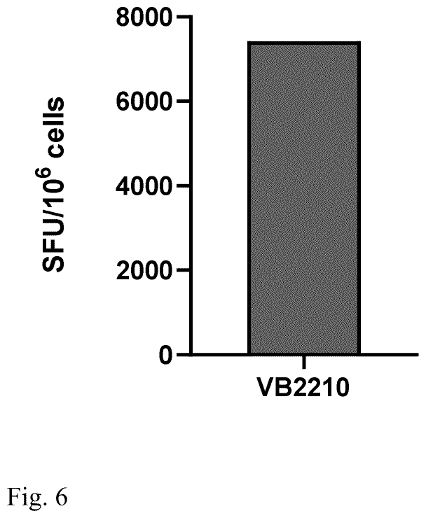

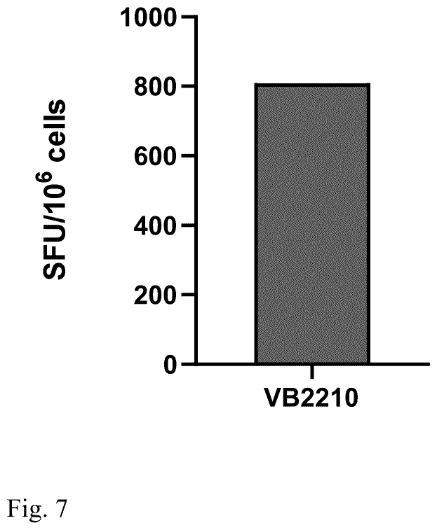

One dose of 25 μg VB2210 construct was also used for vaccination in the C57BL/6 wildtype mouse model. 14 days post vaccination, in an ELISpot assay using peptide pools composed of overlapping 15-mer peptides covering all the epitopes/groups of epitopes included in the construct, the results showed that VB2210 induced a strong specific T cell response (FIG. 6). The strength of the responses differed between the two wildtype mouse models, BALB/c (FIG. 6, 3163 SFU/10⁶ cells) and C57BL/6 (FIG. 7, 7414 SFU/10⁶ cells), which is expected and due to the different genetic background of the two mouse strains which express distinct MHC haplotypes. When performing an ELISpot assay using peptides and peptide pools composed of peptides corresponding to human HLA-A2.1 epitopes with splenocytes from C57BL/6 wildtype mice vaccinated with VB2210, the T cell response is lower (FIG. 7), which is expected as the number of HLA-A2.1 specific epitopes in VB2210 is low compared to the total number of 96 epitopes, used for stimulation when performing the assay with 15-mer overlapping peptide pools covering all epitopes/groups of epitopes present in the VB2210 construct.

Taken together, vaccination with a single dose of 25 or 50 μg of any of the VB10.COV2 constructs induced strong and consistent specific T cell responses across multiple epitopes in three mouse models, 14 days after vaccination.

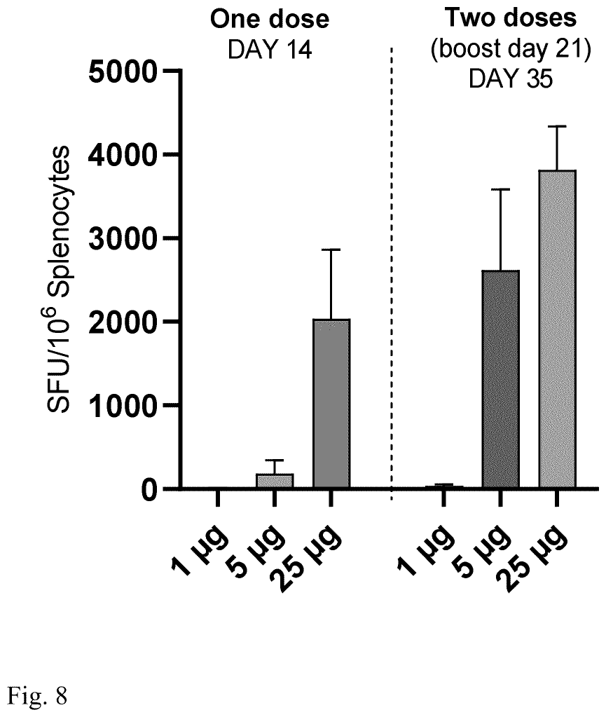

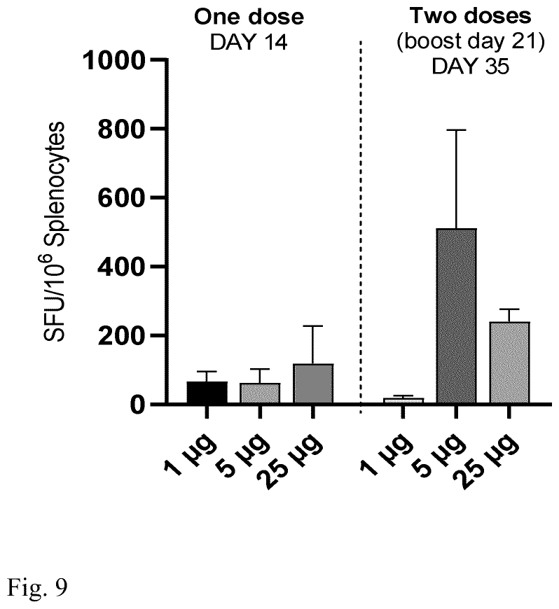

Furthermore, the responses induced by VB2210 at three dose levels, 1, 5 and 25 μg, were evaluated in C57BL/6-Mcph1Tg(HLA-A2.1)1Enge/J transgenic mice in either a one or two dose regimen. The second dose (boost vaccination) was administered 21 days after the first vaccination. The cellular responses against specific T cells epitopes from SARS-CoV-2 included in the construct were assessed in the splenocytes from individual mice 14 days post first vaccination (if only one dose was administered) or 14 days post the boost vaccination (if 2 doses were administered). Peptide pools composed of overlapping 15-mer peptides covering all the epitopes/groups of epitopes (FIG. 8) or peptides and peptide pools composed of peptides corresponding to human HLA-A2.1 epitopes (FIG. 9) included in the construct were used in the ELISpot assay. The results showed a dose-dependent immune response with stronger responses induced by 25 μg of VB2210 in comparison to the lower doses (dose response). The results showed further that the second vaccination at day 21 considerably enhanced the T cell responses, revealing a significant role of boosting (FIGS. 8 and 9). Again, as expected, the overall T cell responses are lower when using peptides corresponding to human HLA-A2.1 epitopes in the assay (FIG. 9) compared to when performing stimulation with 15-mer overlapping peptide pools covering all epitopes/groups of epitopes present in the VB2210 construct (FIG. 8).

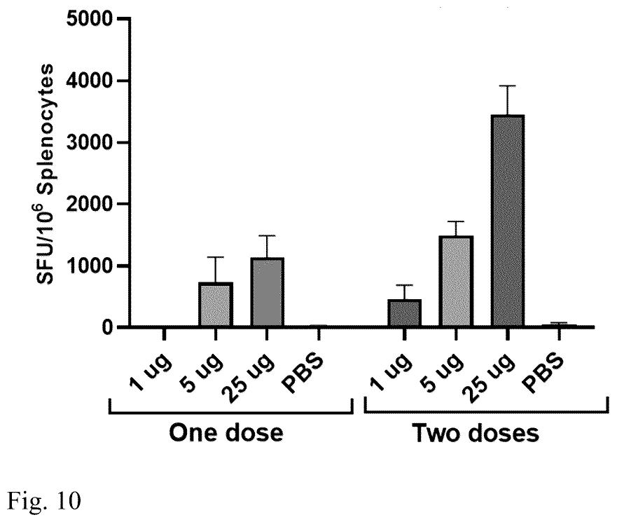

The responses induced by VB2210 at three dose levels, 1, 5 and 25 μg, were also evaluated in C57BL/6 wildtype mice in either a one or two dose regimen. The second dose (boost vaccination) was administered 21 days after the first vaccination. The cellular responses against specific T cells epitopes from SARS-CoV-2 included in the construct were assessed in the splenocytes from individual mice 84 days post vaccination (if only one dose was administered) or 85 days post vaccination (if 2 doses were administered). Selected peptide pools composed of overlapping 15-mer peptides/peptides covering multiple epitopes/groups of epitopes included in the construct were used in the ELISpot assay. The results shown in FIG. 10 show that a single dose of 5 or 25 μg of VB2210 induced long-lasting T cell responses. Boost vaccination at day 21 increased the strength of the immunity evoked by 5 or 25 μg doses. The persistent T cell immunity detected up to 12 weeks posts vaccination indicates established immunological memory.

### Example 4

### Induction of Multifunctional CD8+ Cells Specific to T Cell Epitopes Contained in VB2210 DNA Constructs in Transgenic HLA-A2.1 Mice

### Cell Stimulation and Staining for Flow Cytometry:

C57BL/6-Mcph1Tg(HLA-A2.1)1Enge/J Transgenic Mice were Vaccinated with One or two doses (boost vaccination at day 21) of 25 μg of VB2210. Splenocytes from vaccinated mice were harvested 14 days after single and boost vaccination, respectively, pooled in groups (n=4), and stimulated with 6 μg/mL of a peptide pool composed of immunogenic peptides identified in the previously carried out ELISpot assays corresponding to multiple HLA-A2.1 epitopes present in the construct. Stimulated cells were stained for phenotyping and intracellular expression of TNF-α, IFN-γ, IL-4, IL-17, IL-2 and FoxP3, and further subjected to multiparameter functional analysis by flow cytometry.

### Results:

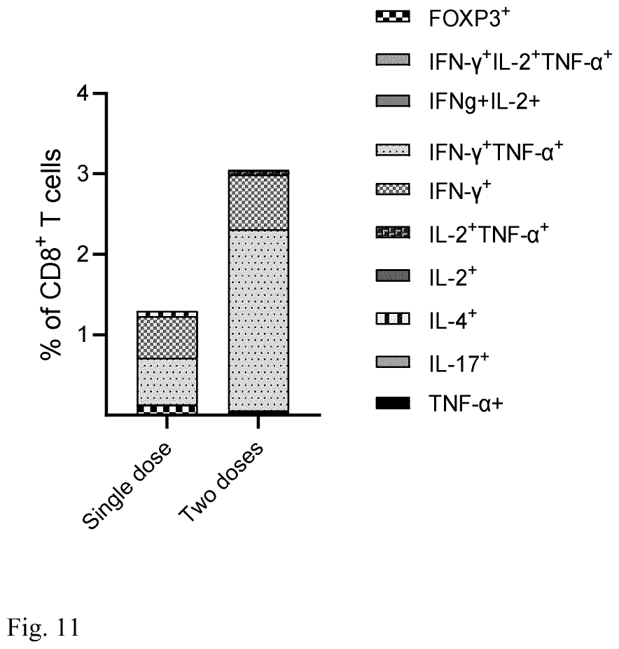

Flow cytometry analysis of T cells showed CD8+ responses specific to the T cell epitopes in VB2210 (FIG. 11). The population of specific CD8+ T cells increased after the second vaccine dose from 1.3% to 3% of the total splenocyte population, indicating a strong effect of the boost vaccination at day 21. The CD8+ specific T cells were dominated by production of IFN-γ or a combination of IFN-γ and TNF-α, a cytokine profile typical for pro-inflammatory responses, implying that VB2210 may induce a cytotoxic T cell response specific for SARS-CoV-2.

### Conclusions:

The data demonstrate that all the VB10.COV2 DNA constructs induced rapid, strong and, for VB2210, dose-dependent T cell responses in 3 different mouse models. These consistently strong T cell responses indicate the ability of the tested VB10.COV2 constructs to induce T cell responses across diverse MHC/HLA haplotypes. In addition, flow cytometry analysis showed multifunctional T cell responses skewed towards a CD8+ and a pro-inflammatory profile. Thus, all of constructs are promising T cell vaccine candidates for prevention and/or treatment of diseases caused by the SARS CoV2 virus.

### Example 5

Clinical Trial with VB10.2210 DNA Plasmid in Humans

A phase 1/2, open label, dose escalation clinical trial was carried out to determine safety, reactogenicity and immunogenicity in humans.

34 healthy volunteers (age 18-60) who had been previously vaccinated at least 8 weeks prior to participation in the trial with at least 2 doses of approved Covid-19 vaccines Comirnaty (Pfizer/BioNTech) or Spikevax (Moderna) were enrolled into 3 dose level cohorts. Comirnaty and Spikevax comprise mRNA encapsulated into lipid nanoparticles which, upon vaccination, deliver the mRNA into cells to direct transient expression of the full-length SARS-CoV2 spike protein (with two point mutations to lock the protein in a preferred prefusion conformation).

Participants were intramuscularly vaccinated with two doses of either 0.3, 1 or 3 mg of VB10.2210 DNA plasmid (which is identical to the DNA plasmid VB2210 mentioned herein) at day 0 and day 21 (booster).

Ex vivo IFN-γ ELISpot assays and ex vivo intracellular staining by flow cytometry were performed to determine cell immunogenicity:

### Ex Vivo IFN-γ ELISpot Assay:

The IFN-γ ELISpot assay was performed on peripheral blood mononuclear cells (PBMCs) of participants vaccinated with VB10.2210 plasmid according to the manufacturer's instructions (Mabtech AB, Sweden). The stimulation was carried out with 5 pools of peptides covering epitopes included in VB10.2210 from the viral proteins S, N, M, ORF7 and ORF1/3/10. Peptides composed of overlapping 15-mer peptides covering all the epitopes present in VB10.2210 in addition to a selection of minimal peptides were used. PBMCs only in cell medium with DMSO concentration corresponding to the DMSO concentration used in the peptide pool (<0.5% DMSO) was used as negative control. The stimulated PBMCs were analyzed for IFN-γ responses using the human IFN-γ ELISpot Plus kit (Mabtech AB, Sweden). Spot-forming cells were measured in an IRIS ELISpot reader from Mabtech AB, Sweden.

### Ex Vivo Intracellular Staining (ICS) Flow Cytometry

PBMCs from VB10.2210-vaccinated participants were stimulated with corresponding peptides as for ELISpot divided into three peptide pools (M+N, ORFs, and S). PBMCs were peptide-stimulated for 16 h prior to intracellular staining for phenotype markers (CD4 and CD8), TNF-α and IFN-γ and subjected to multiparameter analysis by flow cytometry.

### Results:

### Safety:

VB10.2210 was safe and well tolerated up the highest dose of 3 mg with no dose-limiting adverse events. No fatal or serious adverse events and no adverse events of any special interest were observed or reported. Local reactions were mainly mild injection site reactions (pain, tenderness and bruises). Systemic reactions were mainly mild or moderate and of short duration. There were no signs of increase in adverse events or reactogenicity after the second dose.

### Cellular Immunogenicity:

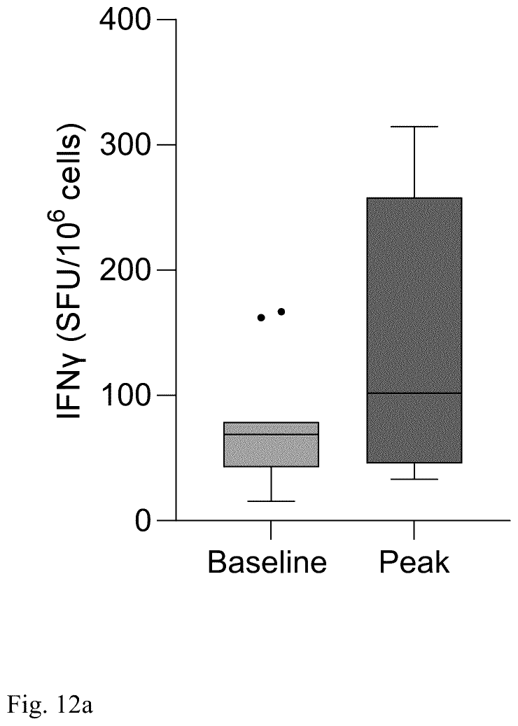

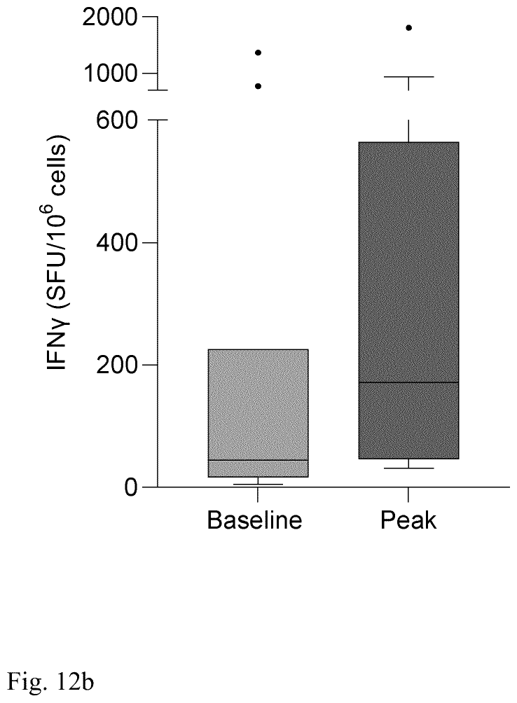

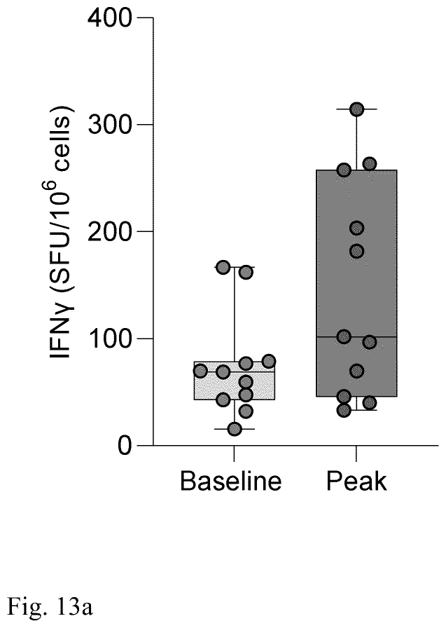

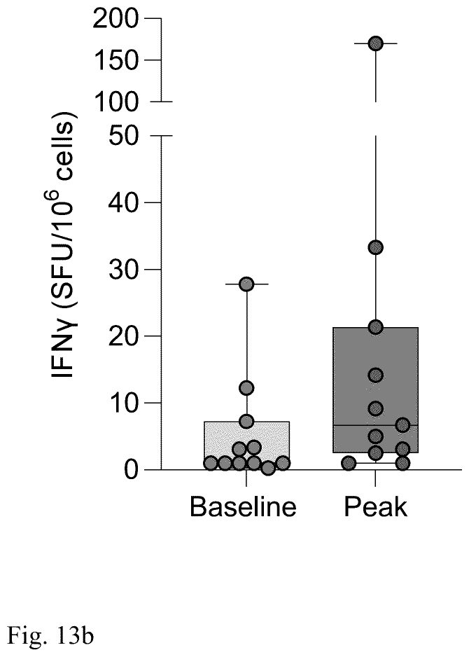

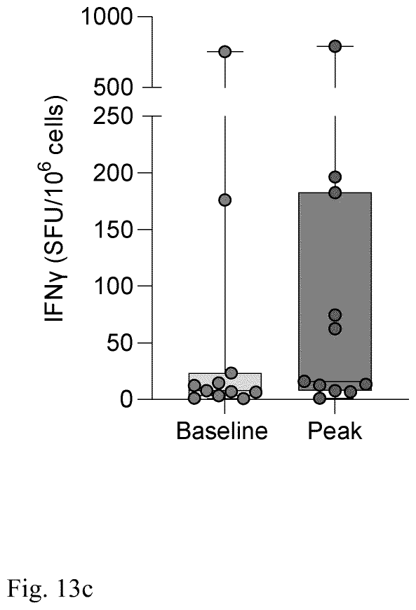

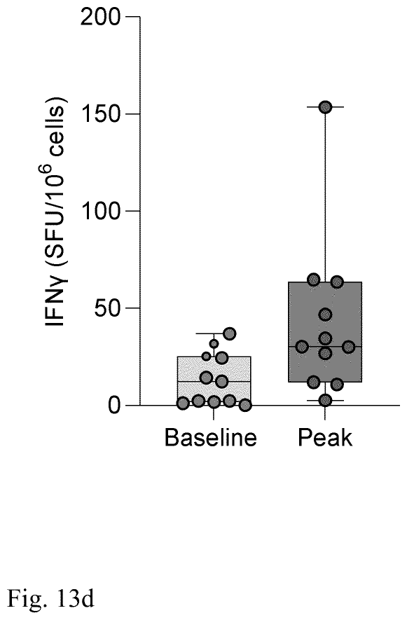

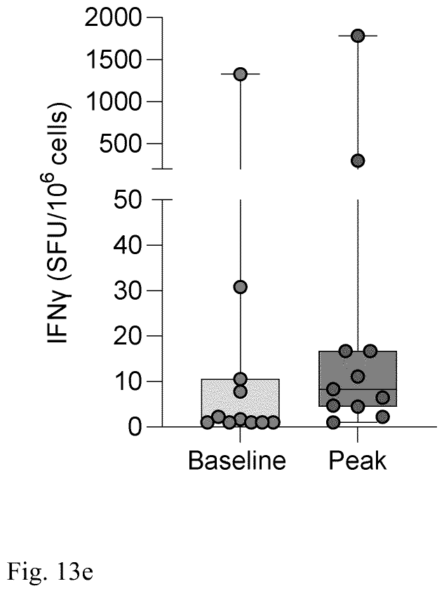

VB10.2210 amplified T cell responses to the Sars-CoV-2 spike protein and induced novel T cell responses against epitopes of all of the seven non-spike Sars-CoV2 proteins (M, N, ORF1ab, ORF3a, ORF7a, ORF7b and ORF10). Responses were dose-dependent with the highest and broadest response observed in the group having received the highest dose (3 mg) (FIGS. 12 and 13).

82% (9 out of 11) participants vaccinated with 3 mg VB10.2210 responded to at least 1 peptide pool (Table 7) and participants showed a response towards all peptide pools (Table 8), demonstrating that the selected epitopes are promiscuous in this study population. 2 participants responded to all peptide pools. A “responder” was defined as showing a response in ex vivo ELISpot of ≥2-fold increase in IFN-γ ISFU/10⁶ PBMCs from baseline (pre-vaccination) in a post-vaccination sample (day 28 or day 35).

TABLE 7

| | Number (%) of responders | |
|---|---|---|
| | in 3 mg dose level | |
| Response to number peptide | group evaluable for | |
| pools | immunogenicity (n = 11) | |
| 0 | 2 | (18%) |
| (no response to any peptide pool) | | |
| 1 | 2 | (18%) |
| 2 | 3 | (28%) |
| 3 | 1 | (9%) |
| 4 | 1 | (9%) |
| 5 | 2 | (18%) |

TABLE 8

| Peptide Pool | Rate of responders (%) in 3 mg dose level group |
|---|---|
| Spike (S) | 16% |
| Membrane (M) | 20% |
| Nucleocapsid (N) | 16% |
| ORF1/3/10 | 24% |
| ORF7 | 27% |

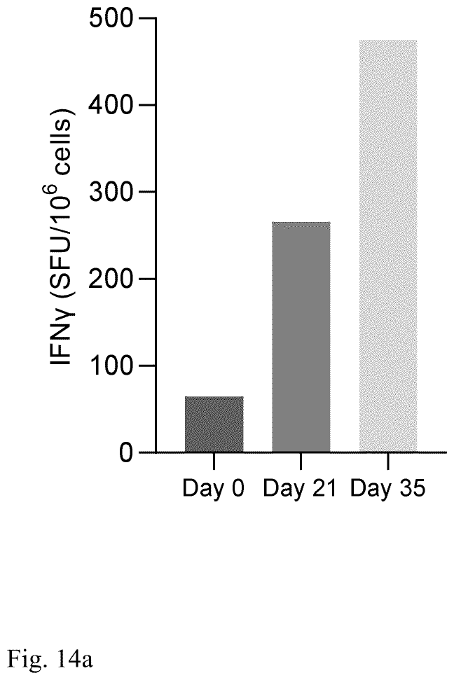

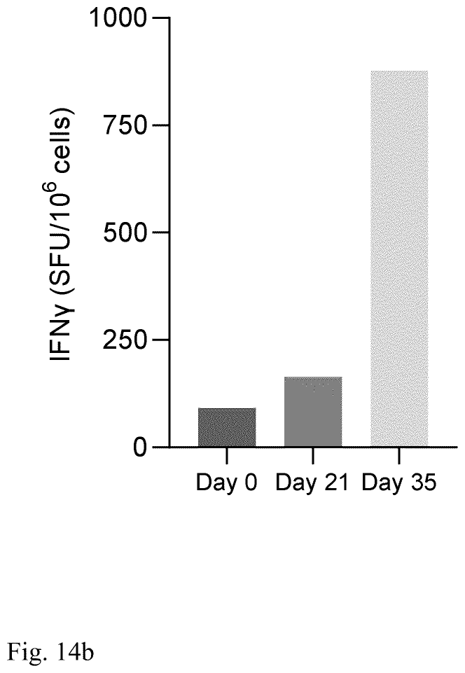

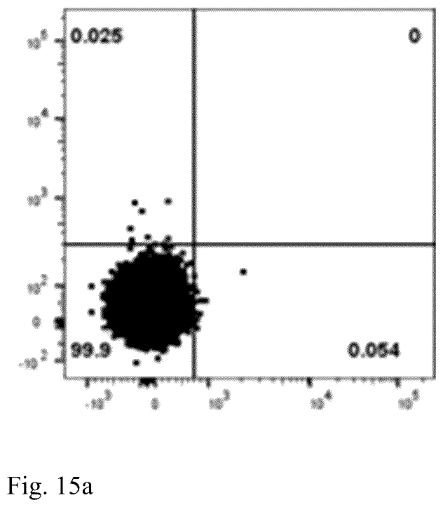

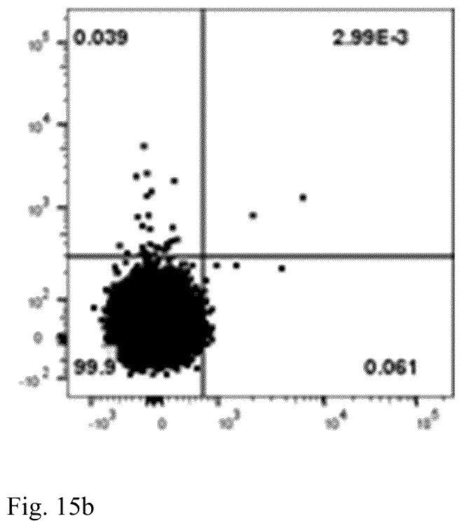

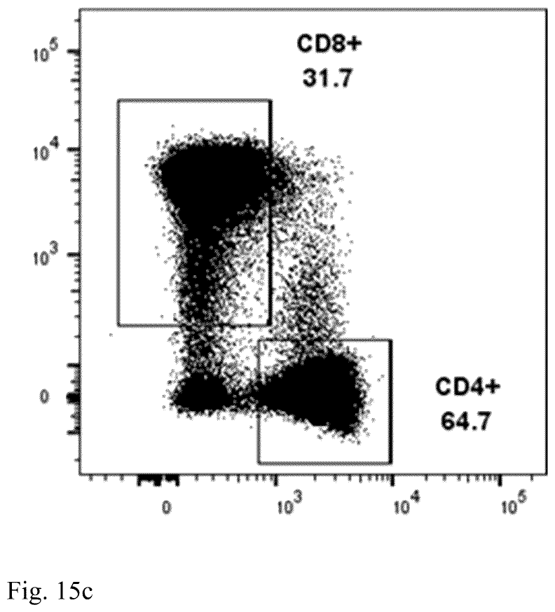

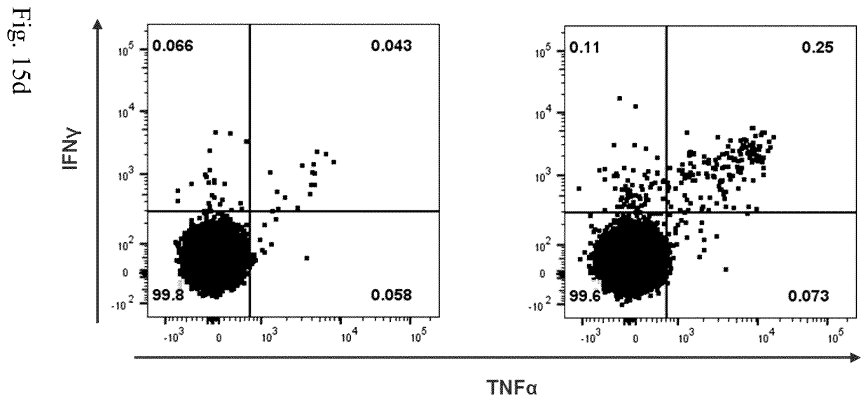

Participants showed a strong T cell response after the first vaccination and T cell responses were further boosted following the second vaccination (FIG. 14). T cell responses were dominated by CD8+ T cells expressing IFN-γ and TNF-αL (FIG. 15).

### Conclusion:

The data demonstrate that vaccination of healthy volunteers with VB10.2210 DNA plasmid resulted in broad T cell responses that are dominated by CD8+ T cell responses. The vaccine was well tolerated with a favorable safety profile.

Sequences

SEQ ID NO: 161
Antigenic unit of VB2193
SEHDYQIGGYTEKWESGVKDCVVLHSYFTSDYYQLYSTQLSTDTGVEHVTFFI
GGGGSGGGGSLPNNTASWFTALTQHGKEDLKFPRGQGVPINTNSSPDDQIGYY
RRATRRIRGGSSGGGSSGGGHLRIAGHHLGRCDIKDLPKEITVATSRTLSYYKL
GASQRVAGDSGGGSSGGGSSGSTQLSTDTGVEHVTFFIYNKIVDEPEGGGGSG
GGGSDGKMKDLSPRWYFYYLGTGPEAGGGGSGGGGSVEGFNCYFPLQSYGF
QPTNGVGGGGSGGGGSKRFDNPVLPFNDGVYFASTEKSNIIRGSSGGGSSGGG
FGMSRIGMEVTPSGTWLTYTGAIKLDDKDPNFKDQVILLNKHIDAYKTFPPTE
GGGGSGGGGSAGAAAYYVGYLQPRTFLLKYNENGSSGGGSSGGGPSIISNEKQ
EILGTVSWNLREMLAHGGSGGGGSGGEGNSPFHPLADNKFALTCFSTQFAFAC
PDGVKHVYQLRARSVSPKLFIGGGGSGGGGSMFVFLVLLPLVSSQCVNLTTRT
QLPPAYTNSFTRGVYYPDKVFRSSVLHSTQDLFLPFFSSGGGGSGGGGKKRWQ
LALSKGVHFVCNLLLLSGGGGSGGGGFIRQEEVQELYSPIFLIVAAIVFIGGGGS
GGGGSYIIKLIFLWLLWPVTLACFVLAAVYRINWGGGGSGGGGSMIELSLIDFY
LCFLAFLLFLVLIMLIIFWFSSGGGGSGGGGLLLVAAGLEAPFLYLYALVYFLQ
SINFVRIIMRLWLCWKGGGGSGGGGSMGYINVFAFPFTIYSLLLCRMNGGGGS
GGGGSSFNPETNILLNVPLHGTILTRPLLESELVIGAVILRGHLRIAGHHGGGGS
GGGGSIILFLALITLATCELYHYQECVRGTTVGGGGSGGGGSKDFGGFNFSQIL
PDPSKPSKRSFIEDLLFNKVTLADAGFIKQYGDCLGDIAARDLICAQKFGGGGS
GGGGSMLAKALRKVPTDNYITTYPGQGLGSGGGGSGGGQTSNFRVQPTESIV
RFPNITNLCPFGEVENATRFASVYAWNRKRISNCVADYSVLYNSASFSTFKGG
GGSGGGGSFYSKWYIRVGARKSAPLIELCVDEAGSKSPIQYIDIGNYSGGGGSG
GGGAFEYYHTTDPSFLGRYMSALNGGGGSGGGGSMESEFRVYSSANNCTFEY
VSQPFLMDLEGKQGNFKNLREFVFKNIDGYFKIYSKHTPINLVRDLPQGSSGG
GSSGGGICGDSTECSNLLLQYGSFCTQLNRALTGIAVEQDKNTQEVFAQVKQI
YKTPPIKDFGGFNFGGGGSGGGGSCLYRNRDVDTDFVNEFYAYLRKHFGGGG
SGGGGSKLPDDFTGCVIAWNSNNLDSKVGGNYNYLYRLFRKSNLKPFERDIST
EIYQAGSTPCNGVEGGGGSGGGGSWKCRSKNPLLYDANYFLCWHTNCYD
SEQ ID NO: 162
Antigenic unit of VB2194
SEHDYQIGGYTEKWESGVKDCVVLHSYFTSGGGGGGGGNLDSKVGGNYNY
LYRLFRKSNLKPFERDISTEIYQAGSTPCNGVEGGGGSGGGGSWKCRSKNPLL
YDANYFLCWHTNCYDGGGGSGGGGSHLRIAGHHLGRCDIKDLPKEITVATSR
TLSYYKLGASQRVAGDSGSSGGGSSGGGKLPDDFTGCVIAWNSNNLDSKVGG
NYNYLSGGGGSGGGGDGKMKDLSPRWYFYYLGTGPEAGGSGGGGSGGQGN
FKNLREFVFKNIDGYFKIYSKHTPINLVRDLPQGSSGGGSSGGGVEGFNCYFPL
QSYGFQPTNGVGGGGSGGGGSFGMSRIGMEVTPSGTWLTYTGAIKLDDKDPN
FKDQVIGGGGSGGGGSFYSKWYIRVGARKSAPLIELCVDEAGSKSPIQYIDIGN
YSGGGGSGGGGLDDKDPNFKDQVILLNKHIDAYKTFPPTEGGGGSGGGGSQT
SNFRVQPTESIVRFPNITNLCPFGEVENATRSGGGGSGGGGMLAKALRKVPTD
NYITTYPGQGLGSGGGGSGGGPPAYTNSFTRGVYYPDKVFRSSVLHSTQDLFL
PFFSSGGGGSGGGGKDFGGFNFSQILPDPSKPSKRSFIEDLLFNKVTLADAGGG
GSGGGGSIILFLALITLATCELYHYQECVRGTTVGGGGSGGGGSKKRWQLALS
KGVHFVCNLLLLSGGGGSGGGGMGYINVFAFPFTIYSLLLCRMNGGGGSGGG
GSLLLVAAGLEAPFLYLYALVYFLQSINFVRIIMRLWLCWKGGGGSGGGGSMI
ELSLIDFYLCFLAFLLFLVLIMLIIFWFSSGGGGSGGGGYIIKLIFLWLLWPVTLA
CFVLAAVYRINWGGGGSGGGGSFIRQEEVQELYSPIFLIVAAIVFIGGGGSGGG
GSMFVFLVLLPLVSSQCVNLTTRTQLPPAYTNSFTRGVGGGGSGGGGSSFNPE
TNILLNVPLHGTILTRPLLESELVIGAVILRGHLRIAGHHGGGGSGGGGSEGNSP
FHPLADNKFALTCFSTQFAFACPDGVKHVYQLRARSVSPKLFIGGGGSGGGGS
EDLLFNKVTLADAGFIKQYGDCLGDIAARDLICAQKFGGGGSGGGGSPSIISNE
KQEILGTVSWNLREMLAHSGGGGSGGGGALTGIAVEQDKNTQEVFAQVKQIY
KTPPIKDFGGFNFGGGGSGGGGSAGAAAYYVGYLQPRTFLLKYNENGSSGGG
SSGGGLPNNTASWFTALTQHGKEDLKFPRGQGVPGGGGSGGGGSKRFDNPVL
PFNDGVYFASTEKSNIIRGSSGGGSSGGGFNATRFASVYAWNRKRISNCVADY
SVLYNSASFSTFKGGGGSGGGGSAFEYYHTTDPSFLGRYMSALNGGGGSGGG
GSMESEFRVYSSANNCTFEYVSQPFLMDLEGKQGNFKNLREFVFKGGGGSGG
GGSSTQLSTDTGVEHVTFFIYNKIVDEPEGGGGSGGGGSCLYRNRDVDTDFVN
EFYAYLRKHFGGGGSGGGGSGVKDCVVLHSYFTSDYYQLYSTQLSTDTGVEH
VTFFIGGGGSGGGGSICGDSTECSNLLLQYGSFCTQLNRALTGIAVEQDKNTSG
GGGSGGGGKEDLKFPRGQGVPINTNSSPDDQIGYYRRATRRIRGG
SEQ ID NO: 163
Antigenic unit of VB2196
SEHDYQIGGYTEKWESGVKDCVVLHSYFTSGGGGSGGGGICGDSTECSNLLL
QYGSFCTQLNRALTGIAVEQDKNTGSGGGGSGGGGVKDCVVLHSYFTSDYYQ
LYSTQLSTDTGVEHVTFFIGGGGSGGGGSCLYRNRDVDTDFVNEFYAYLRKHF
GGGGSGGGGSDGKMKDLSPRWYFYYLGTGPEAGGSGGGGSGGQGNFKNLRE
FVFKNIDGYFKIYSKHTPINLVRDLPQGSSGGGSSGGGFGMSRIGMEVTPSGTW
LTYTGAIKLDDKDPNFKDQVIGGGGSGGGGSFYSKWYIRVGARKSAPLIELCV
DEAGSKSPIQYIDIGNYSGGGGSGGGGLPNNTASWFTALTQHGKEDLKFPRGQ
GVPGGGGSGGGGSALTGIAVEQDKNTQEVFAQVKQIYKTPPIKDFGGFNFGGG
GSGGGGSPSIISNEKQEILGTVSWNLREMLAHSGGGGSGGGGKDFGGFNFSQIL
PDPSKPSKRSFIEDLLFNKVTLADAGGGGSGGGGSEGNSPFHPLADNKFALTCF
STQFAFACPDGVKHVYQLRARSVSPKLFIGGGGSGGGGSSFNPETNILLNVPLH
GTILTRPLLESELVIGAVILRGHLRIAGHHGGGGSGGGGSMFVFLVLLPLVSSQ
CVNLTTRTQLPPAYTNSFTRGVGGGGSGGGGSFIRQEEVQELYSPIFLIVAAIVF
IGGGGSGGGGSYIIKLIFLWLLWPVTLACFVLAAVYRINWGGGGSGGGGSMIE
LSLIDFYLCFLAFLLFLVLIMLIIFWFSSGGGGSGGGGLLLVAAGLEAPFLYLYA
LVYFLQSINFVRIIMRLWLCWKGGGGSGGGGSMGYINVFAFPFTIYSLLLCRM
NGGGGSGGGGSKKRWQLALSKGVHFVCNLLLLSGGGGSGGGGIILFLALITLA
TCELYHYQECVRGTTVGGGGSGGGGSEDLLFNKVTLADAGFIKQYGDCLGDI
AARDLICAQKFGGGGSGGGGSPPAYTNSFTRGVYYPDKVFRSSVLHSTQDLFL
PFFSSGGGGSGGGGMLAKALRKVPTDNYITTYPGQGLGGGSGGGGSGAGAAA
YYVGYLQPRTFLLKYNENGSSGGGSSGGGLDDKDPNFKDQVILLNKHIDAYK
TFPPTEGGGGSGGGGSKRFDNPVLPFNDGVYFASTEKSNIIRGSSGGGSSGGGA
FEYYHTTDPSFLGRYMSALNGGGGSGGGGSMESEFRVYSSANNCTFEYVSQPF
LMDLEGKQGNFKNLREFVFKGGGGSGGGGSSTQLSTDTGVEHVTFFIYNKIVD
EPEGGGGSGGGGSHLRIAGHHLGRCDIKDLPKEITVATSRTLSYYKLGASQRV
AGDSGSSGGGSSGGGWKCRSKNPLLYDANYFLCWHTNCYDGGGGSGGGGSK
EDLKFPRGQGVPINTNSSPDDQIGYYRRATRRIRGG
SEQ ID NO: 164
Antigenic unit of VB2197
GVKDCVVLHSYFTSDYYQLYSTQLSSGGGGSGGGGKLPDDFTGCVIAWNSNN
LDSKVGGNYNYLYRLFRKSNLKPFERDISTEIYQAGSTPCNGVEGGGGSGGGG
SDGKMKDLSPRWYFYYLGTGPEAGGGGSGGGGSVEGENCYFPLQSYGFQPTN
GVGGGGSGGGGSFGMSRIGMEVTPSGTWLTYTGAIKLDDKDPNFKDQVILLN
KHIDAYKTFPPTEGGGGSGGGGSPSIISNEKQEILGTVSWNLREMLAHGGSGG
GGSGGEGNSPFHPLADNKFALTCFSTQFAFACPDGVKHVYQLRARSVSPKLFI
GGGGSGGGGSMGYINVFAFPFTIYSLLLCRMNGGGGSGGGGSMIELSLIDFYL
CFLAFLLFLVLIMLIIFWFSSGGGGSGGGGLLLVAAGLEAPFLYLYALVYFLQSI
NFVRIIMRLWLCWKGGGGSGGGGSIILFLALITLATCELYHYQECVRGTTVGG
GGSGGGGSKDFGGFNFSQILPDPSKPSKRSFIEDLLFNKVTLADAGFIKQYGDC
LGDIAARDLICAQKFGGGGSGGGGSQTSNFRVQPTESIVRFPNITNLCPFGEVF
NATRFASVYAWNRKRISNCVADYSVLYNSASFSTFKGGGGSGGGGSKRFDNP
VLPFNDGVYFASTEKSNIIRGSSGGGSSGGGAFEYYHTTDPSFLGRYMSALNG
GGGSGGGGSHLRIAGHHLGRCDIKDLPKEITVATSRTLSYYKLGASQRVAGDS
GSSGGGSSGGGLPNNTASWFTALTQHGKEDLKFPRGQGVPINTNSSPDDQIGY
YRRATRRIRGG
SEQ ID NO: 165
Antigenic unit of VB2207
MSFNPETNILLNVPLHGTILTRPLLESELVIGAVILRGHLRIAGHHSGSSGSKDF
GGFNFSQILPDPSKPSKRSFIEDLLFNKVTLADAGFIKQYGDCLGDIAARDLICA
QKFSGSSGSSTQLSTDTGVEHVTFFIYNKIVDEPESGSSGSMESEFRVYSSANNC
TFEYVSQPFLMDLEGKQGNFKNLREFVFKNIDGYFKIYSKHTPINLVRDLPQGS
GSSGSAFEYYHTTDPSFLGRYMSALNSGSSGSLLLVAAGLEAPFLYLYALVYF
LQSINFVRIIMRLWLCWKSGSSGSFIRQEEVQELYSPIFLIVAAIVFISGSSGSSEH
DYQIGGYTEKWESGVKDCVVLHSYFTSDYYQLYSTQLSTDTGVEHVTFFIGGS
GGYIIKLIFLWLLWPVTLACFVLAAVYRINWGGSGGCLYRNRDVDTDFVNEF
YAYLRKHFGGSGGKRFDNPVLPFNDGVYFASTEKSNIIRGSGSSGSFYSKWYIR
VGARKSAPLIELCVDEAGSKSPIQYIDIGNYGGSGGMIELSLIDFYLCFLAFLLFL
VLIMLIIFWFSGGSGGICGDSTECSNLLLQYGSFCTQLNRALTGIAVEQDKNTQ
EVFAQVKQIYKTPPIKDFGGFNFSGSSGSHLRIAGHHLGRCDIKDLPKEITVATS
RTLSYYKLGASQRVAGDSGGGSGGDGKMKDLSPRWYFYYLGTGPEAGGSGG
PSIISNEKQEILGTVSWNLREMLAHGGSGGWKCRSKNPLLYDANYFLCWHTN
CYDGGSGGKKRWQLALSKGVHFVCNLLLLSGSSGSMLAKALRKVPTDNYITT
YPGQGLSGSSGSMGYINVFAFPFTIYSLLLCRMNGGSGGMFVFLVLLPLVSSQC
VNLTTRTQLPPAYTNSFTRGVYYPDKVFRSSVLHSTQDLFLPFFSGGSGGAGA
AAYYVGYLQPRTFLLKYNENGGGSGGEGNSPFHPLADNKFALTCFSTQFAFA
CPDGVKHVYQLRARSVSPKLFIGGSGGFGMSRIGMEVTPSGTWLTYTGAIKLD
DKDPNFKDQVILLNKHIDAYKTFPPTESGSSGSIILFLALITLATCELYHYQECV
RGTTVSGSSGSLPNNTASWFTALTQHGKEDLKFPRGQGVPINTNSSPDDQIGY
YRRATRRIRGGSGSSGS
SEQ ID NO: 166
Antigenic unit of VB2208
MCLYRNRDVDTDFVNEFYAYLRKHFSGSSGSKDFGGFNFSQILPDPSKPSKRSF
IEDLLFNKVTLADASGSSGSAFEYYHTTDPSFLGRYMSALNGGSGGEDLLFNK
VTLADAGFIKQYGDCLGDIAARDLICAQKFGGSGGICGDSTECSNLLLQYGSFC
TQLNRALTGIAVEQDKNTGGSGGDGKMKDLSPRWYFYYLGTGPEAGGSGGK
RFDNPVLPFNDGVYFASTEKSNIIRGSGSSGSMESEFRVYSSANNCTFEYVSQPF
LMDLEGKQGNFKNLREFVFKSGSSGSMIELSLIDFYLCFLAFLLFLVLIMLIIFW
FSSGSSGSFIRQEEVQELYSPIFLIVAAIVFISGSSGSKKRWQLALSKGVHFVCNL
LLLSGSSGSIILFLALITLATCELYHYQECVRGTTVGGSGGMFVFLVLLPLVSSQ
CVNLTTRTQLPPAYTNSFTRGVGGSGGSFNPETNILLNVPLHGTILTRPLLESEL
VIGAVILRGHLRIAGHHGGSGGMLAKALRKVPTDNYITTYPGQGLSGSSGSLL
LVAAGLEAPFLYLYALVYFLQSINFVRIIMRLWLCWKGGSGGLDDKDPNFKD
QVILLNKHIDAYKTFPPTEGGSGGYIIKLIFLWLLWPVTLACFVLAAVYRINWS
GSSGSQGNFKNLREFVFKNIDGYFKIYSKHTPINLVRDLPQGSGSSGSPPAYTNS
FTRGVYYPDKVFRSSVLHSTQDLFLPFFSSGSSGSGVKDCVVLHSYFTSDYYQL
YSTQLSTDTGVEHVTFFISGSSGSALTGIAVEQDKNTQEVFAQVKQIYKTPPIK
DFGGFNFGGSGGAGAAAYYVGYLQPRTFLLKYNENGSGSSGSFYSKWYIRVG
ARKSAPLIELCVDEAGSKSPIQYIDIGNYGGSGGWKCRSKNPLLYDANYFLCW
HTNCYDGGSGGMGYINVFAFPFTIYSLLLCRMNSGSSGSLPNNTASWFTALTQ
HGKEDLKFPRGQGVPGGSGGFGMSRIGMEVTPSGTWLTYTGAIKLDDKDPNF
KDQVISGSSGSEGNSPFHPLADNKFALTCFSTQFAFACPDGVKHVYQLRARSV
SPKLFIGGSGGSEHDYQIGGYTEKWESGVKDCVVLHSYFTSGSSGSKEDLKFP
RGQGVPINTNSSPDDQIGYYRRATRRIRGGGGSGGSTQLSTDTGVEHVTFFIYN
KIVDEPEGGSGGHLRIAGHHLGRCDIKDLPKEITVATSRTLSYYKLGASQRVAG
DSGGGSGGPSIISNEKQEILGTVSWNLREMLAHSGSSGS
SEQ ID NO: 167
Antigenic unit of VB2210
MFGMSRIGMEVTPSGTWLTYTGAIKLDDKDPNFKDQVIGGSGGQTSNFRVQP
TESIVRFPNITNLCPFGEVENATRSGSSGSMGYINVFAFPFTIYSLLLCRMNGGS
GGFNATRFASVYAWNRKRISNCVADYSVLYNSASFSTFKSGSSGSNLDSKVGG
NYNYLYRLFRKSNLKPFERDISTEIYQAGSTPCNGVESGSSGSVEGFNCYFPLQ
SYGFQPTNGVGGSGGLDDKDPNFKDQVILLNKHIDAYKTFPPTESGSSGSGVK
DCVVLHSYFTSDYYQLYSTQLSSGSSGSEGNSPFHPLADNKFALTCFSTQFAFA
CPDGVKHVYQLRARSVSPKLFISGSSGSDGKMKDLSPRWYFYYLGTGPEASGS
SGSMIELSLIDFYLCFLAFLLFLVLIMLIIFWFSGGSGGAFEYYHTTDPSFLGRY
MSALNSGSSGSKLPDDFTGCVIAWNSNNLDSKVGGNYNYLGGSGGKRFDNPV
LPFNDGVYFASTEKSNIIRGGGSGGLLLVAAGLEAPFLYLYALVYFLQSINFVRI
IMRLWLCWKGGSGGEDLLFNKVTLADAGFIKQYGDCLGDIAARDLICAQKFG
GSGGIILFLALITLATCELYHYQECVRGTTVSGSSGSHLRIAGHHLGRCDIKDLP
KEITVATSRTLSYYKLGASQRVAGDSGGGSGGLPNNTASWFTALTQHGKEDL
KFPRGQGVPGGSGGKDFGGFNFSQILPDPSKPSKRSFIEDLLFNKVTLADASGS
SGSKEDLKFPRGQGVPINTNSSPDDQIGYYRRATRRIRGGSGSSGSPSIISNEKQ
EILGTVSWNLREMLAHGGSGG
SEQ ID NO: 168
Amino acid sequence of signal peptide of human MIP1-α (CCL3L1) (amino acids 1-23),
human MIP1-α (CCL3L1) (amino acids 24-93), hinge exon h1 from IgG3 (amino acids
94-105), hinge exon h4 from IgG3 (amino acids 106-120), a dimerization unit
linker (amino acids 121-130), the human CH3 domain of IgG3 (amino acids 131-237)
and a unit linker (238-242):
M¹QVSTAALAVLLCTMALCNQVLS²³A²⁴PLAADTPTACCFSYTSRQIPQNFIAD
YFETSSQCSKPSVIFLTKRGRQVCADPSEEWVQKYVSDLELSA⁹³E⁹⁴LKTPLGDT
THT¹⁰⁵E¹⁰⁶PKSCDTPPPCPRCP¹²⁰G¹²¹GGSSGGGSG¹³⁰G¹³¹QPREPQVYTLPPSREE
MTKNQVSLTCLVKGFYPSDIAVEWESSGQPENNYNTTPPMLDSDGSFFLYSKL
TVDKSRWQQGNIFSCSVMHEALHNRFTQKSLSLSPGK²³⁷GLGGL²⁴²
SEQ ID NO: 169
Amino acid sequence of anti-pan HLA class II with Ig VH signal peptide (amino
acids 1-19), anti-pan HLA class II VL (amino acids 20-127), a linker (amino acids
128-142) and anti-pan HLA class II VH (amino acids 143-260):
M¹NFGLRLIFLVLTLKGVQC¹⁹D²⁰IQMTQTTSSLSASLGDRVTISCSASQDINNYL
NWYQQKPDGTVKLLIYYTSSLHSGVPSRFSGSGSGTDYSLTISNLEPEDIATYY
CQQYSKFPRTFGGGTKLEIKR¹²⁷G¹²⁸GGGSGGGGSGGGGS¹⁴²Q¹⁴³IQLVQSGPEL
KKPGETVKISCKASGYTFINYGMNWVKQTPGKGLKWMGWINTYSGEPTYPD
DFKGRFAFSLETSASTAYLQLNNLKNEDMATYFCARGDYYGPFDNWGQGTTL
TVSS²⁶⁰
SEQ ID NO: 170
Amino acid sequence of human TPA signal peptide:
MDAMKRGLCCVLLLCGAVFVSP
SEQ ID NO: 171
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLSEHDYQIGGYTEKWESGVKDCVVLHSYFTSDYYQLYSTQLSTDTGVE
HVTFFIGGGGSGGGGSLPNNTASWFTALTQHGKEDLKFPRGQGVPINTNSSPD
DQIGYYRRATRRIRGGSSGGGSSGGGHLRIAGHHLGRCDIKDLPKEITVATSRT
LSYYKLGASQRVAGDSGGGSSGGGSSGSTQLSTDTGVEHVTFFIYNKIVDEPE
GGGGSGGGGSDGKMKDLSPRWYFYYLGTGPEAGGGGSGGGGSVEGFNCYFP
LQSYGFQPTNGVGGGGSGGGGSKRFDNPVLPFNDGVYFASTEKSNIIRGSSGG
GSSGGGFGMSRIGMEVTPSGTWLTYTGAIKLDDKDPNFKDQVILLNKHIDAYK
TFPPTEGGGGSGGGGSAGAAAYYVGYLQPRTFLLKYNENGSSGGGSSGGGPSI
ISNEKQEILGTVSWNLREMLAHGGSGGGGSGGEGNSPFHPLADNKFALTCFST
QFAFACPDGVKHVYQLRARSVSPKLFIGGGGSGGGGSMFVFLVLLPLVSSQCV
NLTTRTQLPPAYTNSFTRGVYYPDKVFRSSVLHSTQDLFLPFFSSGGGGSGGGG
KKRWQLALSKGVHFVCNLLLLSGGGGSGGGGFIRQEEVQELYSPIFLIVAAIVF
IGGGGSGGGGSYIIKLIFLWLLWPVTLACFVLAAVYRINWGGGGSGGGGSMIE
LSLIDFYLCFLAFLLFLVLIMLIIFWFSSGGGGSGGGGLLLVAAGLEAPFLYLYA
LVYFLQSINFVRIIMRLWLCWKGGGGSGGGGSMGYINVFAFPFTIYSLLLCRM
NGGGGSGGGGSSFNPETNILLNVPLHGTILTRPLLESELVIGAVILRGHLRIAGH
HGGGGSGGGGSIILFLALITLATCELYHYQECVRGTTVGGGGSGGGGSKDFGG
FNFSQILPDPSKPSKRSFIEDLLFNKVTLADAGFIKQYGDCLGDIAARDLICAQK
FGGGGSGGGGSMLAKALRKVPTDNYITTYPGQGLGSGGGGSGGGQTSNFRVQ
PTESIVRFPNITNLCPFGEVFNATRFASVYAWNRKRISNCVADYSVLYNSASFS
TFKGGGGSGGGGSFYSKWYIRVGARKSAPLIELCVDEAGSKSPIQYIDIGNYSG
GGGSGGGGAFEYYHTTDPSFLGRYMSALNGGGGSGGGGSMESEFRVYSSAN
NCTFEYVSQPFLMDLEGKQGNFKNLREFVFKNIDGYFKIYSKHTPINLVRDLPQ
GSSGGGSSGGGICGDSTECSNLLLQYGSFCTQLNRALTGIAVEQDKNTQEVFA
QVKQIYKTPPIKDFGGFNFGGGGSGGGGSCLYRNRDVDTDFVNEFYAYLRKH
FGGGGSGGGGSKLPDDFTGCVIAWNSNNLDSKVGGNYNYLYRLFRKSNLKPF
ERDISTEIYQAGSTPCNGVEGGGGSGGGGSWKCRSKNPLLYDANYFLCWHTN
CYD
SEQ ID NO: 172
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLSEHDYQIGGYTEKWESGVKDCVVLHSYFTSGGGGSGGGGNLDSKVG
GNYNYLYRLFRKSNLKPFERDISTEIYQAGSTPCNGVEGGGGSGGGGSWKCRS
KNPLLYDANYFLCWHTNCYDGGGGSGGGGSHLRIAGHHLGRCDIKDLPKEIT
VATSRTLSYYKLGASQRVAGDSGSSGGGSSGGGKLPDDFTGCVIAWNSNNLD
SKVGGNYNYLSGGGGSGGGGDGKMKDLSPRWYFYYLGTGPEAGGSGGGGS
GGQGNFKNLREFVFKNIDGYFKIYSKHTPINLVRDLPQGSSGGGSSGGGVEGF
NCYFPLQSYGFQPTNGVGGGGSGGGGSFGMSRIGMEVTPSGTWLTYTGAIKL
DDKDPNFKDQVIGGGGSGGGGSFYSKWYIRVGARKSAPLIELCVDEAGSKSPI
QYIDIGNYSGGGGSGGGGLDDKDPNFKDQVILLNKHIDAYKTFPPTEGGGGSG
GGGSQTSNFRVQPTESIVRFPNITNLCPFGEVENATRSGGGGSGGGGMAKAL
RKVPTDNYITTYPGQGLGSGGGGSGGGPPAYTNSFTRGVYYPDKVFRSSVLHS
TQDLFLPFFSSGGGGSGGGGKDFGGFNFSQILPDPSKPSKRSFIEDLLFNKVTLA
DAGGGGSGGGGSIILFLALITLATCELYHYQECVRGTTVGGGGSGGGGSKKR
WQLALSKGVHFVCNLLLLSGGGGSGGGGMGYINVFAFPFTIYSLLLCRMNGG
GGSGGGGSLLLVAAGLEAPFLYLYALVYFLQSINFVRIIMRLWLCWKGGGGS
GGGGSMIELSLIDFYLCFLAFLLFLVLIMLIIFWFSSGGGGSGGGGYIIKLIFLWL
LWPVTLACFVLAAVYRINWGGGGSGGGGSFIRQEEVQELYSPIFLIVAAIVFIG
GGGSGGGGSMFVFLVLLPLVSSQCVNLTTRTQLPPAYTNSFTRGVGGGGSGG
GGSSFNPETNILLNVPLHGTILTRPLLESELVIGAVILRGHLRIAGHHGGGGSGG
GGSEGNSPFHPLADNKFALTCFSTQFAFACPDGVKHVYQLRARSVSPKLFIGG
GGSGGGGSEDLLFNKVTLADAGFIKQYGDCLGDIAARDLICAQKFGGGGSGG
GGSPSIISNEKQEILGTVSWNLREMLAHSGGGGSGGGGALTGIAVEQDKNTQE
VFAQVKQIYKTPPIKDFGGFNFGGGGSGGGGSAGAAAYYVGYLQPRTFLLKY
NENGSSGGGSSGGGLPNNTASWFTALTQHGKEDLKFPRGQGVPGGGGSGGG
GSKRFDNPVLPFNDGVYFASTEKSNIIRGSSGGGSSGGGFNATRFASVYAWNR
KRISNCVADYSVLYNSASFSTFKGGGGSGGGGSAFEYYHTTDPSFLGRYMSAL
NGGGGSGGGGSMESEFRVYSSANNCTFEYVSQPFLMDLEGKQGNFKNLREFV
FKGGGGSGGGGSSTQLSTDTGVEHVTFFIYNKIVDEPEGGGGSGGGGSCLYRN
RDVDTDFVNEFYAYLRKHFGGGGSGGGGSGVKDCVVLHSYFTSDYYQLYST
QLSTDTGVEHVTFFIGGGGSGGGGSICGDSTECSNLLLQYGSFCTQLNRALTGI
AVEQDKNTSGGGGSGGGGKEDLKFPRGQGVPINTNSSPDDQIGYYRRATRRIR
GG
SEQ ID NO: 173
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLSEHDYQIGGYTEKWESGVKDCVVLHSYFTSGGGGSGGGGICGDSTEC
SNLLLQYGSFCTQLNRALTGIAVEQDKNTGSGGGGSGGGGVKDCVVLHSYFT
SDYYQLYSTQLSTDTGVEHVTFFIGGGGSGGGGSCLYRNRDVDTDFVNEFYA
YLRKHFGGGGSGGGGSDGKMKDLSPRWYFYYLGTGPEAGGSGGGGSGGQG
NFKNLREFVFKNIDGYFKIYSKHTPINLVRDLPQGSSGGGSSGGGFGMSRIGME
VTPSGTWLTYTGAIKLDDKDPNFKDQVIGGGGSGGGGSFYSKWYIRVGARKS
APLIELCVDEAGSKSPIQYIDIGNYSGGGGSGGGGLPNNTASWFTALTQHGKE
DLKFPRGQGVPGGGGSGGGGSALTGIAVEQDKNTQEVFAQVKQIYKTPPIKDF
GGFNFGGGGSGGGGSPSIISNEKQEILGTVSWNLREMLAHSGGGGSGGGGKDF
GGFNFSQILPDPSKPSKRSFIEDLLFNKVTLADAGGGGSGGGGSEGNSPFHPLA
DNKFALTCFSTQFAFACPDGVKHVYQLRARSVSPKLFIGGGGSGGGGSSFNPE
TNILLNVPLHGTILTRPLLESELVIGAVILRGHLRIAGHHGGGGSGGGGSMFVFL
VLLPLVSSQCVNLTTRTQLPPAYTNSFTRGVGGGGSGGGGSFIRQEEVQELYSP
IFLIVAAIVFIGGGGSGGGGSYIIKLIFLWLLWPVTLACFVLAAVYRINWGGGGS
GGGGSMIELSLIDFYLCFLAFLLFLVLIMLIIFWFSSGGGGSGGGGLLLVAAGLE
APFLYLYALVYFLQSINFVRIIMRLWLCWKGGGGSGGGGSMGYINVFAFPFTI
YSLLLCRMNGGGGSGGGGSKKRWQLALSKGVHFVCNLLLLSGGGGSGGGGII
LFLALITLATCELYHYQECVRGTTVGGGGSGGGGSEDLLFNKVTLADAGFIKQ
YGDCLGDIAARDLICAQKFGGGGSGGGGSPPAYTNSFTRGVYYPDKVFRSSVL
HSTQDLFLPFFSSGGGGSGGGGMLAKALRKVPTDNYITTYPGQGLGGGSGGG
GSGAGAAAYYVGYLQPRTFLLKYNENGSSGGGSSGGGLDDKDPNFKDQVILL
NKHIDAYKTFPPTEGGGGSGGGGSKRFDNPVLPFNDGVYFASTEKSNIIRGSSG
GGSSGGGAFEYYHTTDPSFLGRYMSALNGGGGSGGGGSMESEFRVYSSANNC
TFEYVSQPFLMDLEGKQGNFKNLREFVFKGGGGSGGGGSSTQLSTDTGVEHV
TFFIYNKIVDEPEGGGGSGGGGSHLRIAGHHLGRCDIKDLPKEITVATSRTLSY
YKLGASQRVAGDSGSSGGGSSGGGWKCRSKNPLLYDANYFLCWHTNCYDGG
GGSGGGGSKEDLKFPRGQGVPINTNSSPDDQIGYYRRATRRIRGG
SEQ ID NO: 174
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLGVKDCVVLHSYFTSDYYQLYSTQLSSGGGGSGGGGKLPDDFTGCVIA
WNSNNLDSKVGGNYNYLYRLFRKSNLKPFERDISTEIYQAGSTPCNGVEGGG
GSGGGGSDGKMKDLSPRWYFYYLGTGPEAGGGGSGGGGSVEGENCYFPLQS
YGFQPTNGVGGGGSGGGGSFGMSRIGMEVTPSGTWLTYTGAIKLDDKDPNFK
DQVILLNKHIDAYKTFPPTEGGGGSGGGGSPSIISNEKQEILGTVSWNLREMLA
HGGSGGGGSGGEGNSPFHPLADNKFALTCFSTQFAFACPDGVKHVYQLRARS
VSPKLFIGGGGSGGGGSMGYINVFAFPFTIYSLLLCRMNGGGGSGGGGSMIELS
LIDFYLCFLAFLLFLVLIMLIIFWFSSGGGGSGGGGLLLVAAGLEAPFLYLYALV
YFLQSINFVRIIMRLWLCWKGGGGSGGGGSIILFLALITLATCELYHYQECVRG
TTVGGGGSGGGGSKDFGGFNFSQILPDPSKPSKRSFIEDLLFNKVTLADAGFIK
QYGDCLGDIAARDLICAQKFGGGGSGGGGSQTSNFRVQPTESIVRFPNITNLCP
FGEVFNATRFASVYAWNRKRISNCVADYSVLYNSASFSTFKGGGGSGGGGSK
RFDNPVLPFNDGVYFASTEKSNIIRGSSGGGSSGGGAFEYYHTTDPSFLGRYMS
ALNGGGGSGGGGSHLRIAGHHLGRCDIKDLPKEITVATSRTLSYYKLGASQRV
AGDSGSSGGGSSGGGLPNNTASWFTALTQHGKEDLKFPRGQGVPINTNSSPDD
QIGYYRRATRRIRGG
SEQ ID NO: 175
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLMSFNPETNILLNVPLHGTILTRPLLESELVIGAVILRGHLRIAGHHSGSS
GSKDFGGFNFSQILPDPSKPSKRSFIEDLLFNKVTLADAGFIKQYGDCLGDIAAR
DLICAQKFSGSSGSSTQLSTDTGVEHVTFFIYNKIVDEPESGSSGSMESEFRVYS
SANNCTFEYVSQPFLMDLEGKQGNFKNLREFVFKNIDGYFKIYSKHTPINLVR
DLPQGSGSSGSAFEYYHTTDPSFLGRYMSALNSGSSGSLLLVAAGLEAPFLYL
YALVYFLQSINFVRIIMRLWLCWKSGSSGSFIRQEEVQELYSPIFLIVAAIVFISG
SSGSSEHDYQIGGYTEKWESGVKDCVVLHSYFTSDYYQLYSTQLSTDTGVEH
VTFFIGGSGGYIIKLIFLWLLWPVTLACFVLAAVYRINWGGSGGCLYRNRDVD
TDFVNEFYAYLRKHFGGSGGKRFDNPVLPFNDGVYFASTEKSNIIRGSGSSGSF
YSKWYIRVGARKSAPLIELCVDEAGSKSPIQYIDIGNYGGSGGMIELSLIDFYLC
FLAFLLFLVLIMLIIFWFSGGSGGICGDSTECSNLLLQYGSFCTQLNRALTGIAV
EQDKNTQEVFAQVKQIYKTPPIKDFGGFNFSGSSGSHLRIAGHHLGRCDIKDLP
KEITVATSRTLSYYKLGASQRVAGDSGGGSGGDGKMKDLSPRWYFYYLGTGP
EAGGSGGPSIISNEKQEILGTVSWNLREMLAHGGSGGWKCRSKNPLLYDANYF
LCWHTNCYDGGSGGKKRWQLALSKGVHFVCNLLLLSGSSGSMLAKALRKVP
TDNYITTYPGQGLSGSSGSMGYINVFAFPFTIYSLLLCRMNGGSGGMFVFLVLL
PLVSSQCVNLTTRTQLPPAYTNSFTRGVYYPDKVFRSSVLHSTQDLFLPFFSGG
SGGAGAAAYYVGYLQPRTFLLKYNENGGGSGGEGNSPFHPLADNKFALTCFS
TQFAFACPDGVKHVYQLRARSVSPKLFIGGSGGFGMSRIGMEVTPSGTWLTYT
GAIKLDDKDPNFKDQVILLNKHIDAYKTFPPTESGSSGSIILFLALITLATCELYH
YQECVRGTTVSGSSGSLPNNTASWFTALTQHGKEDLKFPRGQGVPINTNSSPD
DQIGYYRRATRRIRGGSGSSGS
SEQ ID NO: 176
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLMCLYRNRDVDTDFVNEFYAYLRKHFSGSSGSKDFGGFNFSQILPDPSK
PSKRSFIEDLLFNKVTLADASGSSGSAFEYYHTTDPSFLGRYMSALNGGSGGED
LLFNKVTLADAGFIKQYGDCLGDIAARDLICAQKFGGSGGICGDSTECSNLLLQ
YGSFCTQLNRALTGIAVEQDKNTGGSGGDGKMKDLSPRWYFYYLGTGPEAG
GSGGKRFDNPVLPFNDGVYFASTEKSNIIRGSGSSGSMESEFRVYSSANNCTFE
YVSQPFLMDLEGKQGNFKNLREFVFKSGSSGSMIELSLIDFYLCFLAFLLFLVLI
MLIIFWFSSGSSGSFIRQEEVQELYSPIFLIVAAIVFISGSSGSKKRWQLALSKGV
HFVCNLLLLSGSSGSIILFLALITLATCELYHYQECVRGTTVGGSGGMFVFLVL
LPLVSSQCVNLTTRTQLPPAYTNSFTRGVGGSGGSFNPETNILLNVPLHGTILTR
PLLESELVIGAVILRGHLRIAGHHGGSGGMLAKALRKVPTDNYITTYPGQGLS
GSSGSLLLVAAGLEAPFLYLYALVYFLQSINFVRIIMRLWLCWKGGSGGLDDK
DPNFKDQVILLNKHIDAYKTFPPTEGGSGGYIIKLIFLWLLWPVTLACFVLAAV
YRINWSGSSGSQGNFKNLREFVFKNIDGYFKIYSKHTPINLVRDLPQGSGSSGS
PPAYTNSFTRGVYYPDKVFRSSVLHSTQDLFLPFFSSGSSGSGVKDCVVLHSYF
TSDYYQLYSTQLSTDTGVEHVTFFISGSSGSALTGIAVEQDKNTQEVFAQVKQI
YKTPPIKDFGGFNFGGSGGAGAAAYYVGYLQPRTFLLKYNENGSGSSGSFYSK
WYIRVGARKSAPLIELCVDEAGSKSPIQYIDIGNYGGSGGWKCRSKNPLLYDA
NYFLCWHTNCYDGGSGGMGYINVFAFPFTIYSLLLCRMNSGSSGSLPNNTAS
WFTALTQHGKEDLKFPRGQGVPGGSGGFGMSRIGMEVTPSGTWLTYTGAIKL
DDKDPNFKDQVISGSSGSEGNSPFHPLADNKFALTCFSTQFAFACPDGVKHVY
QLRARSVSPKLFIGGSGGSEHDYQIGGYTEKWESGVKDCVVLHSYFTSGSSGS
KEDLKFPRGQGVPINTNSSPDDQIGYYRRATRRIRGGGGSGGSTQLSTDTGVE
HVTFFIYNKIVDEPEGGSGGHLRIAGHHLGRCDIKDLPKEITVATSRTLSYYKL
GASQRVAGDSGGGSGGPSIISNEKQEILGTVSWNLREMLAHSGSSGS
SEQ ID NO: 177
APLAADTPTACCFSYTSRQIPQNFIADYFETSSQCSKPSVIFLTKRGRQVCADPS
EEWVQKYVSDLELSAELKTPLGDTTHTEPKSCDTPPPCPRCPGGGSSGGGSGG
QPREPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLSPGK
GLGGLMFGMSRIGMEVTPSGTWLTYTGAIKLDDKDPNFKDQVIGGSGGQTSN
FRVQPTESIVRFPNITNLCPFGEVENATRSGSSGSMGYINVFAFPFTIYSLLLCRM
NGGSGGFNATRFASVYAWNRKRISNCVADYSVLYNSASFSTFKSGSSGSNLDS
KVGGNYNYLYRLFRKSNLKPFERDISTEIYQAGSTPCNGVESGSSGSVEGENCY
FPLQSYGFQPTNGVGGSGGLDDKDPNFKDQVILLNKHIDAYKTFPPTESGSSGS
GVKDCVVLHSYFTSDYYQLYSTQLSSGSSGSEGNSPFHPLADNKFALTCFSTQF
AFACPDGVKHVYQLRARSVSPKLFISGSSGSDGKMKDLSPRWYFYYLGTGPE
ASGSSGSMIELSLIDFYLCFLAFLLFLVLIMLIIFWFSGGSGGAFEYYHTTDPSFL
GRYMSALNSGSSGSKLPDDFTGCVIAWNSNNLDSKVGGNYNYLGGSGGKRF
DNPVLPFNDGVYFASTEKSNIIRGGGSGGLLLVAAGLEAPFLYLYALVYFLQSI
NFVRIIMRLWLCWKGGSGGEDLLFNKVTLADAGFIKQYGDCLGDIAARDLICA
QKFGGSGGIILFLALITLATCELYHYQECVRGTTVSGSSGSHLRIAGHHLGRCDI
KDLPKEITVATSRTLSYYKLGASQRVAGDSGGGSGGLPNNTASWFTALTQHG
KEDLKFPRGQGVPGGSGGKDFGGFNFSQILPDPSKPSKRSFIEDLLFNKVTLAD
ASGSSGSKEDLKFPRGQGVPINTNSSPDDQIGYYRRATRRIRGGSGSSGSPSIISN
EKQEILGTVSWNLREMLAHGGSGG
SEQ ID NO: 178
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLSEHDYQIGGYTEKWESGVKDCVV
LHSYFTSDYYQLYSTQLSTDTGVEHVTFFIGGGGSGGGGSLPNNTASWFTALT
QHGKEDLKFPRGQGVPINTNSSPDDQIGYYRRATRRIRGGSSGGGSSGGGHLRI
AGHHLGRCDIKDLPKEITVATSRTLSYYKLGASQRVAGDSGGGSSGGGSSGST
QLSTDTGVEHVTFFIYNKIVDEPEGGGGSGGGGSDGKMKDLSPRWYFYYLGT
GPEAGGGGSGGGGSVEGENCYFPLQSYGFQPTNGVGGGGSGGGGSKRFDNPV
LPFNDGVYFASTEKSNIIRGSSGGGSSGGGFGMSRIGMEVTPSGTWLTYTGAIK
LDDKDPNFKDQVILLNKHIDAYKTFPPTEGGGGSGGGGSAGAAAYYVGYLQP
RTFLLKYNENGSSGGGSSGGGPSIISNEKQEILGTVSWNLREMLAHGGSGGGG
SGGEGNSPFHPLADNKFALTCFSTQFAFACPDGVKHVYQLRARSVSPKLFIGG
GGSGGGGSMFVFLVLLPLVSSQCVNLTTRTQLPPAYTNSFTRGVYYPDKVFRS
SVLHSTQDLFLPFFSSGGGGSGGGGKKRWQLALSKGVHFVCNLLLLSGGGGS
GGGGFIRQEEVQELYSPIFLIVAAIVFIGGGGSGGGGSYIIKLIFLWLLWPVTLAC
FVLAAVYRINWGGGGSGGGGSMIELSLIDFYLCFLAFLLFLVLIMLIIFWFSSGG
GGSGGGGLLLVAAGLEAPFLYLYALVYFLQSINFVRIIMRLWLCWKGGGGSG
GGGSMGYINVFAFPFTIYSLLLCRMNGGGGSGGGGSSFNPETNILLNVPLHGTI
LTRPLLESELVIGAVILRGHLRIAGHHGGGGSGGGGSIILFLALITLATCELYHY
QECVRGTTVGGGGSGGGGSKDFGGFNFSQILPDPSKPSKRSFIEDLLFNKVTLA
DAGFIKQYGDCLGDIAARDLICAQKFGGGGSGGGGSMLAKALRKVPTDNYIT
TYPGQGLGSGGGGSGGGQTSNFRVQPTESIVRFPNITNLCPFGEVENATRFASV
YAWNRKRISNCVADYSVLYNSASFSTFKGGGGSGGGGSFYSKWYIRVGARKS
APLIELCVDEAGSKSPIQYIDIGNYSGGGGSGGGGAFEYYHTTDPSFLGRYMSA
LNGGGGSGGGGSMESEFRVYSSANNCTFEYVSQPFLMDLEGKQGNFKNLREF
VFKNIDGYFKIYSKHTPINLVRDLPQGSSGGGSSGGGICGDSTECSNLLLQYGSF
CTQLNRALTGIAVEQDKNTQEVFAQVKQIYKTPPIKDFGGFNFGGGGSGGGGS
CLYRNRDVDTDFVNEFYAYLRKHFGGGGSGGGGSKLPDDFTGCVIAWNSNN
LDSKVGGNYNYLYRLFRKSNLKPFERDISTEIYQAGSTPCNGVEGGGGSGGGG
SWKCRSKNPLLYDANYFLCWHTNCYD
SEQ ID NO: 179
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLSEHDYQIGGYTEKWESGVKDCVV
LHSYFTSGGGGSGGGGNLDSKVGGNYNYLYRLFRKSNLKPFERDISTEIYQAG
STPCNGVEGGGGSGGGGSWKCRSKNPLLYDANYFLCWHTNCYDGGGGSGG
GGSHLRIAGHHLGRCDIKDLPKEITVATSRTLSYYKLGASQRVAGDSGSSGGG
SSGGGKLPDDFTGCVIAWNSNNLDSKVGGNYNYLSGGGGSGGGGDGKMKDL
SPRWYFYYLGTGPEAGGSGGGGSGGQGNFKNLREFVFKNIDGYFKIYSKHTPI
NLVRDLPQGSSGGGSSGGGVEGFNCYFPLQSYGFQPTNGVGGGGSGGGGSFG
MSRIGMEVTPSGTWLTYTGAIKLDDKDPNFKDQVIGGGGSGGGGSFYSKWYI
RVGARKSAPLIELCVDEAGSKSPIQYIDIGNYSGGGGSGGGGLDDKDPNFKDQ
VILLNKHIDAYKTFPPTEGGGGSGGGGSQTSNFRVQPTESIVRFPNITNLCPFGE
VFNATRSGGGGSGGGGMLAKALRKVPTDNYITTYPGQGLGSGGGGSGGGPPA
YTNSFTRGVYYPDKVFRSSVLHSTQDLFLPFFSSGGGGSGGGGKDFGGFNFSQI
LPDPSKPSKRSFIEDLLFNKVTLADAGGGGSGGGGSIILFLALITLATCELYHYQ
ECVRGTTVGGGGSGGGGSKKRWQLALSKGVHFVCNLLLLSGGGGSGGGGM
GYINVFAFPFTIYSLLLCRMNGGGGSGGGGSLLLVAAGLEAPFLYLYALVYFL
QSINFVRIIMRLWLCWKGGGGSGGGGSMIELSLIDFYLCFLAFLLFLVLIMLIIF
WFSSGGGGSGGGGYIIKLIFLWLLWPVTLACFVLAAVYRINWGGGGSGGGGS
FIRQEEVQELYSPIFLIVAAIVFIGGGGSGGGGSMFVFLVLLPLVSSQCVNLTTR
TQLPPAYTNSFTRGVGGGGSGGGGSSFNPETNILLNVPLHGTILTRPLLESELVI
GAVILRGHLRIAGHHGGGGSGGGGSEGNSPFHPLADNKFALTCFSTQFAFACP
DGVKHVYQLRARSVSPKLFIGGGGSGGGGSEDLLFNKVTLADAGFIKQYGDC
LGDIAARDLICAQKFGGGGSGGGGSPSIISNEKQEILGTVSWNLREMLAHSGGG
GSGGGGALTGIAVEQDKNTQEVFAQVKQIYKTPPIKDFGGFNFGGGGSGGGGS
AGAAAYYVGYLQPRTFLLKYNENGSSGGGSSGGGLPNNTASWFTALTQHGK
EDLKFPRGQGVPGGGGSGGGGSKRFDNPVLPFNDGVYFASTEKSNIIRGSSGG
GSSGGGFNATRFASVYAWNRKRISNCVADYSVLYNSASFSTFKGGGGSGGGG
SAFEYYHTTDPSFLGRYMSALNGGGGSGGGGSMESEFRVYSSANNCTFEYVS
QPFLMDLEGKQGNFKNLREFVFKGGGGSGGGGSSTQLSTDTGVEHVTFFIYNK
IVDEPEGGGGSGGGGSCLYRNRDVDTDFVNEFYAYLRKHFGGGGSGGGGSGV
KDCVVLHSYFTSDYYQLYSTQLSTDTGVEHVTFFIGGGGSGGGGSICGDSTEC
SNLLLQYGSFCTQLNRALTGIAVEQDKNTSGGGGSGGGGKEDLKFPRGQGVPI
NTNSSPDDQIGYYRRATRRIRGG
SEQ ID NO: 180
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLSEHDYQIGGYTEKWESGVKDCVV
LHSYFTSGGGGSGGGGICGDSTECSNLLLQYGSFCTQLNRALTGIAVEQDKNT
GSGGGGGGGGVKDCVVLHSYFTSDYYQLYSTQLSTDTGVEHVTFFIGGGGS
GGGGSCLYRNRDVDTDFVNEFYAYLRKHFGGGGSGGGGSDGKMKDLSPRW
YFYYLGTGPEAGGSGGGGSGGQGNFKNLREFVFKNIDGYFKIYSKHTPINLVR
DLPQGSSGGGSSGGGFGMSRIGMEVTPSGTWLTYTGAIKLDDKDPNFKDQVIG
GGGSGGGGSFYSKWYIRVGARKSAPLIELCVDEAGSKSPIQYIDIGNYSGGGGS
GGGGLPNNTASWFTALTQHGKEDLKFPRGQGVPGGGGSGGGGSALTGIAVEQ
DKNTQEVFAQVKQIYKTPPIKDFGGFNFGGGGSGGGGSPSIISNEKQEILGTVS
WNLREMLAHSGGGGSGGGGKDFGGFNFSQILPDPSKPSKRSFIEDLLFNKVTL
ADAGGGGSGGGGSEGNSPFHPLADNKFALTCFSTQFAFACPDGVKHVYQLRA
RSVSPKLFIGGGGSGGGGSSFNPETNILLNVPLHGTILTRPLLESELVIGAVILRG
HLRIAGHHGGGGSGGGGSMFVFLVLLPLVSSQCVNLTTRTQLPPAYTNSFTRG
VGGGGSGGGGSFIRQEEVQELYSPIFLIVAAIVFIGGGGSGGGGSYIIKLIFLWLL
WPVTLACFVLAAVYRINWGGGGSGGGGSMIELSLIDFYLCFLAFLLFLVLIMLI
IFWFSSGGGGSGGGGLLLVAAGLEAPFLYLYALVYFLQSINFVRIIMRLWLCW
KGGGGSGGGGSMGYINVFAFPFTIYSLLLCRMNGGGGSGGGGSKKRWQLALS
KGVHFVCNLLLLSGGGGSGGGGIILFLALITLATCELYHYQECVRGTTVGGGG
SGGGGSEDLLFNKVTLADAGFIKQYGDCLGDIAARDLICAQKFGGGGSGGGG
SPPAYTNSFTRGVYYPDKVFRSSVLHSTQDLFLPFFSSGGGGSGGGGMLAKAL
RKVPTDNYITTYPGQGLGGGSGGGGSGAGAAAYYVGYLQPRTFLLKYNENGS
SGGGSSGGGLDDKDPNFKDQVILLNKHIDAYKTFPPTEGGGGSGGGGSKRFDN
PVLPFNDGVYFASTEKSNIIRGSSGGGSSGGGAFEYYHTTDPSFLGRYMSALNG
GGGSGGGGSMESEFRVYSSANNCTFEYVSQPFLMDLEGKQGNFKNLREFVFK
GGGGSGGGGSSTQLSTDTGVEHVTFFIYNKIVDEPEGGGGSGGGGSHLRIAGH
HLGRCDIKDLPKEITVATSRTLSYYKLGASQRVAGDSGSSGGGSSGGGWKCRS
KNPLLYDANYFLCWHTNCYDGGGGSGGGGSKEDLKFPRGQGVPINTNSSPDD
QIGYYRRATRRIRGG
SEQ ID NO: 181
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLGVKDCVVLHSYFTSDYYQLYSTQ
LSSGGGGSGGGGKLPDDFTGCVIAWNSNNLDSKVGGNYNYLYRLFRKSNLKP
FERDISTEIYQAGSTPCNGVEGGGGSGGGGSDGKMKDLSPRWYFYYLGTGPE
AGGGGSGGGGSVEGFNCYFPLQSYGFQPTNGVGGGGSGGGGSFGMSRIGMEV
TPSGTWLTYTGAIKLDDKDPNFKDQVILLNKHIDAYKTFPPTEGGGGSGGGGS
PSIISNEKQEILGTVSWNLREMLAHGGSGGGGSGGEGNSPFHPLADNKFALTCF
STQFAFACPDGVKHVYQLRARSVSPKLFIGGGGSGGGGSMGYINVFAFPFTIYS
LLLCRMNGGGGSGGGGSMIELSLIDFYLCFLAFLLFLVLIMLIIFWFSSGGGGSG
GGGLLLVAAGLEAPFLYLYALVYFLQSINFVRIIMRLWLCWKGGGGSGGGGSI
ILFLALITLATCELYHYQECVRGTTVGGGGSGGGGSKDFGGFNFSQILPDPSKP
SKRSFIEDLLFNKVTLADAGFIKQYGDCLGDIAARDLICAQKFGGGGSGGGGS
QTSNFRVQPTESIVRFPNITNLCPFGEVFNATRFASVYAWNRKRISNCVADYSV
LYNSASFSTFKGGGGSGGGGSKRFDNPVLPFNDGVYFASTEKSNIIRGSSGGGS
SGGGAFEYYHTTDPSFLGRYMSALNGGGGSGGGGSHLRIAGHHLGRCDIKDL
PKEITVATSRTLSYYKLGASQRVAGDSGSSGGGSSGGGLPNNTASWFTALTQH
GKEDLKFPRGQGVPINTNSSPDDQIGYYRRATRRIRGG
SEQ ID NO: 182
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLMSFNPETNILLNVPLHGTILTRPLL
ESELVIGAVILRGHLRIAGHHSGSSGSKDFGGFNFSQILPDPSKPSKRSFIEDLLF
NKVTLADAGFIKQYGDCLGDIAARDLICAQKFSGSSGSSTQLSTDTGVEHVTFF
IYNKIVDEPESGSSGSMESEFRVYSSANNCTFEYVSQPFLMDLEGKQGNFKNLR
EFVFKNIDGYFKIYSKHTPINLVRDLPQGSGSSGSAFEYYHTTDPSFLGRYMSA
LNSGSSGSLLLVAAGLEAPFLYLYALVYFLQSINFVRIIMRLWLCWKSGSSGSFI
RQEEVQELYSPIFLIVAAIVFISGSSGSSEHDYQIGGYTEKWESGVKDCVVLHSY
FTSDYYQLYSTQLSTDTGVEHVTFFIGGSGGYIIKLIFLWLLWPVTLACFVLAA
VYRINWGGSGGCLYRNRDVDTDFVNEFYAYLRKHFGGSGGKRFDNPVLPFN
DGVYFASTEKSNIIRGSGSSGSFYSKWYIRVGARKSAPLIELCVDEAGSKSPIQY
IDIGNYGGSGGMIELSLIDFYLCFLAFLLFLVLIMLIIFWFSGGSGGICGDSTECS
NLLLQYGSFCTQLNRALTGIAVEQDKNTQEVFAQVKQIYKTPPIKDFGGFNFS
GSSGSHLRIAGHHLGRCDIKDLPKEITVATSRTLSYYKLGASQRVAGDSGGGS
GGDGKMKDLSPRWYFYYLGTGPEAGGSGGPSIISNEKQEILGTVSWNLREML
AHGGSGGWKCRSKNPLLYDANYFLCWHTNCYDGGSGGKKRWQLALSKGVH
FVCNLLLLSGSSGSMLAKALRKVPTDNYITTYPGQGLSGSSGSMGYINVFAFPF
TIYSLLLCRMNGGSGGMFVFLVLLPLVSSQCVNLTTRTQLPPAYTNSFTRGVY
YPDKVFRSSVLHSTQDLFLPFFSGGSGGAGAAAYYVGYLQPRTFLLKYNENG
GGSGGEGNSPFHPLADNKFALTCFSTQFAFACPDGVKHVYQLRARSVSPKLFI
GGSGGFGMSRIGMEVTPSGTWLTYTGAIKLDDKDPNFKDQVILLNKHIDAYKT
FPPTESGSSGSIILFLALITLATCELYHYQECVRGTTVSGSSGSLPNNTASWFTAL
TQHGKEDLKFPRGQGVPINTNSSPDDQIGYYRRATRRIRGGSGSSGS
SEQ ID NO: 183
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLMCLYRNRDVDTDFVNEFYAYLRK
HFSGSSGSKDFGGFNFSQILPDPSKPSKRSFIEDLLFNKVTLADASGSSGSAFEY
YHTTDPSFLGRYMSALNGGSGGEDLLFNKVTLADAGFIKQYGDCLGDIAARD
LICAQKFGGSGGICGDSTECSNLLLQYGSFCTQLNRALTGIAVEQDKNTGGSG
GDGKMKDLSPRWYFYYLGTGPEAGGSGGKRFDNPVLPFNDGVYFASTEKSNI
IRGSGSSGSMESEFRVYSSANNCTFEYVSQPFLMDLEGKQGNFKNLREFVFKS
GSSGSMIELSLIDFYLCFLAFLLFLVLIMLIIFWFSSGSSGSFIRQEEVQELYSPIFL
IVAAIVFISGSSGSKKRWQLALSKGVHFVCNLLLLSGSSGSIILFLALITLATCEL
YHYQECVRGTTVGGSGGMFVFLVLLPLVSSQCVNLTTRTQLPPAYTNSFTRGV
GGSGGSFNPETNILLNVPLHGTILTRPLLESELVIGAVILRGHLRIAGHHGGSGG
MLAKALRKVPTDNYITTYPGQGLSGSSGSLLLVAAGLEAPFLYLYALVYFLQS
INFVRIIMRLWLCWKGGSGGLDDKDPNFKDQVILLNKHIDAYKTFPPTEGGSG
GYIIKLIFLWLLWPVTLACFVLAAVYRINWSGSSGSQGNFKNLREFVFKNIDGY
FKIYSKHTPINLVRDLPQGSGSSGSPPAYTNSFTRGVYYPDKVFRSSVLHSTQD
LFLPFFSSGSSGSGVKDCVVLHSYFTSDYYQLYSTQLSTDTGVEHVTFFISGSSG
SALTGIAVEQDKNTQEVFAQVKQIYKTPPIKDFGGFNFGGSGGAGAAAYYVG
YLQPRTFLLKYNENGSGSSGSFYSKWYIRVGARKSAPLIELCVDEAGSKSPIQY
IDIGNYGGSGGWKCRSKNPLLYDANYFLCWHTNCYDGGSGGMGYINVFAFPF
TIYSLLLCRMNSGSSGSLPNNTASWFTALTQHGKEDLKFPRGQGVPGGSGGFG
MSRIGMEVTPSGTWLTYTGAIKLDDKDPNFKDQVISGSSGSEGNSPFHPLADN
KFALTCFSTQFAFACPDGVKHVYQLRARSVSPKLFIGGSGGSEHDYQIGGYTE
KWESGVKDCVVLHSYFTSGSSGSKEDLKFPRGQGVPINTNSSPDDQIGYYRRA
TRRIRGGGGSGGSTQLSTDTGVEHVTFFIYNKIVDEPEGGSGGHLRIAGHHLGR
CDIKDLPKEITVATSRTLSYYKLGASQRVAGDSGGGSGGPSIISNEKQEILGTVS
WNLREMLAHSGSSGS
SEQ ID NO: 184
MQVSTAALAVLLCTMALCNQVLSAPLAADTPTACCFSYTSRQIPQNFIADYFE
TSSQCSKPSVIFLTKRGRQVCADPSEEWVQKYVSDLELSAELKTPLGDTTHTEP
KSCDTPPPCPRCPGGGSSGGGSGGQPREPQVYTLPPSREEMTKNQVSLTCLVK
GFYPSDIAVEWESSGQPENNYNTTPPMLDSDGSFFLYSKLTVDKSRWQQGNIF
SCSVMHEALHNRFTQKSLSLSPGKGLGGLMFGMSRIGMEVTPSGTWLTYTGAI
KLDDKDPNFKDQVIGGSGGQTSNFRVQPTESIVRFPNITNLCPFGEVENATRSG
SSGSMGYINVFAFPFTIYSLLLCRMNGGSGGFNATRFASVYAWNRKRISNCVA
DYSVLYNSASFSTFKSGSSGSNLDSKVGGNYNYLYRLFRKSNLKPFERDISTEI
YQAGSTPCNGVESGSSGSVEGFNCYFPLQSYGFQPTNGVGGSGGLDDKDPNF
KDQVILLNKHIDAYKTFPPTESGSSGSGVKDCVVLHSYFTSDYYQLYSTQLSSG
SSGSEGNSPFHPLADNKFALTCFSTQFAFACPDGVKHVYQLRARSVSPKLFISG
SSGSDGKMKDLSPRWYFYYLGTGPEASGSSGSMIELSLIDFYLCFLAFLLFLVLI
MLIIFWFSGGSGGAFEYYHTTDPSFLGRYMSALNSGSSGSKLPDDFTGCVIAW
NSNNLDSKVGGNYNYLGGSGGKRFDNPVLPFNDGVYFASTEKSNIIRGGGSG
GLLLVAAGLEAPFLYLYALVYFLQSINFVRIIMRLWLCWKGGSGGEDLLFNKV
TLADAGFIKQYGDCLGDIAARDLICAQKFGGSGGIILFLALITLATCELYHYQE
CVRGTTVSGSSGSHLRIAGHHLGRCDIKDLPKEITVATSRTLSYYKLGASQRVA
GDSGGGSGGLPNNTASWFTALTQHGKEDLKFPRGQGVPGGSGGKDFGGFNFS
QILPDPSKPSKRSFIEDLLFNKVTLADASGSSGSKEDLKFPRGQGVPINTNSSPD
DQIGYYRRATRRIRGGSGSSGSPSIISNEKQEILGTVSWNLREMLAHGGSGG
SEQ ID NO: 185
Nucleotide sequence of VB2210
ATGCAGGTCTCCACTGCTGCCCTTGCCGTCCTCCTCTGCACCATGGCTCTCT
GCAACCAGGTCCTCTCTGCACCACTTGCTGCTGACACGCCGACCGCCTGCT
GCTTCAGCTACACCTCCCGACAGATTCCACAGAATTTCATAGCTGACTACT
TTGAGACGAGCAGCCAGTGCTCCAAGCCCAGTGTCATCTTCCTAACCAAGA
GAGGCCGGCAGGTCTGTGCTGACCCCAGTGAGGAGTGGGTCCAGAAATAC
GTCAGTGACCTGGAGCTGAGTGCCGAGCTCAAAACCCCACTTGGTGACAC
AACTCACACAGAGCCCAAATCTTGTGACACACCTCCCCCGTGCCCAAGGTG
CCCAGGCGGTGGAAGCAGCGGAGGTGGAAGTGGAGGACAGCCCCGAGAA
CCACAGGTGTACACCCTGCCCCCATCCCGGGAGGAGATGACCAAGAACCA
GGTCAGCCTGACCTGCCTGGTCAAAGGCTTCTACCCCAGCGACATCGCCGT
GGAGTGGGAGAGCAGCGGGCAGCCGGAGAACAACTACAACACCACGCCTC
CCATGCTGGACTCCGACGGCTCCTTCTTCCTCTACAGCAAGCTCACCGTGG
ACAAGAGCAGGTGGCAGCAGGGGAACATCTTCTCATGCTCCGTGATGCAT
GAGGCTCTGCACAACCGCTTCACGCAGAAGAGCCTCTCCCTGTCTCCGGGT
AAAGGCCTCGGTGGCCTGATGTTCGGCATGTCACGGATAGGTATGGAAGTC
ACGCCGAGTGGAACATGGCTAACTTATACTGGGGCCATAAAACTTGATGA
CAAGGATCCCAATTTTAAGGACCAAGTGATCGGCGGCAGCGGTGGACAGA
CCTCCAACTTCAGAGTGCAACCAACAGAGTCTATCGTTAGGTTCCCTAACA
TTACTAACCTATGTCCCTTTGGAGAGGTGTTCAACGCTACACGCTCGGGAA
GTTCTGGCAGTATGGGATATATTAACGTCTTCGCTTTTCCGTTCACGATCTA
TAGCCTATTGCTCTGTAGGATGAACGGTGGTTCCGGTGGCTTTAACGCGAC
ACGATTCGCGTCGGTCTATGCATGGAACCGTAAACGCATCTCTAACTGCGT
TGCAGATTATAGTGTGCTCTACAACAGCGCGTCCTTCAGTACCTTTAAGTC
GGGCAGCTCAGGGAGCAACCTCGACAGTAAAGTAGGTGGTAATTATAACT
ACCTGTATCGCTTATTCCGCAAGAGTAATCTCAAGCCGTTTGAGCGAGACA
TATCCACGGAAATCTACCAAGCTGGGTCTACCCCATGTAATGGCGTAGAAT
CAGGCTCTTCAGGTTCGGTCGAAGGATTCAATTGTTACTTTCCTTTACAAAG
CTATGGATTCCAGCCAACTAACGGGGTGGGCGGTAGTGGAGGGCTAGACG
ACAAAGACCCTAATTTCAAAGATCAGGTGATACTCCTCAACAAGCACATTG
ATGCCTACAAGACCTTTCCACCAACCGAGAGTGGGTCGTCGGGTTCGGGCG
TAAAAGATTGCGTCGTGTTACATTCTTACTTCACGTCCGACTACTACCAGTT
GTACTCGACTCAGCTTAGTAGCGGTTCTTCCGGCAGTGAAGGTAACTCGCC
CTTTCACCCTTTAGCCGACAATAAATTCGCATTAACTTGTTTTAGTACTCAA
TTTGCGTTCGCGTGTCCAGACGGAGTGAAACATGTGTATCAGTTACGGGCA
AGAAGTGTCTCTCCTAAACTATTCATCTCAGGATCATCCGGGAGCGATGGC
AAAATGAAGGACTTGAGTCCACGCTGGTACTTCTACTATTTGGGCACAGGA
CCAGAAGCATCGGGGAGTTCAGGGTCCATGATCGAACTCAGTCTTATCGAT
TTCTATCTATGTTTTCTCGCCTTCCTTCTATTTCTAGTCCTAATCATGCTCAT
CATCTTTTGGTTCTCCGGCGGTTCAGGCGGAGCATTCGAATACTATCATAC
AACGGACCCATCGTTCCTAGGCCGGTATATGAGCGCACTTAACAGTGGCTC
GTCCGGGTCAAAACTGCCTGACGACTTCACCGGGTGCGTCATCGCCTGGAA
CTCTAATAACCTGGATAGCAAAGTGGGAGGCAATTACAATTACTTAGGCG
GGTCTGGCGGGAAACGGTTTGACAATCCCGTGCTGCCATTCAATGATGGAG
TGTATTTCGCTAGTACAGAAAAGTCCAATATCATCAGGGGCGGCGGAAGT
GGCGGTCTGCTCTTAGTGGCCGCTGGATTAGAAGCGCCGTTCTTATATCTG
TATGCCCTCGTTTATTTCCTGCAGTCAATCAACTTTGTGCGGATTATTATGC
GGCTGTGGTTGTGTTGGAAGGGAGGGTCGGGTGGAGAAGACCTGCTTTTCA
ATAAGGTGACCTTAGCTGATGCGGGTTTCATTAAGCAGTACGGCGATTGTC
TTGGCGACATTGCAGCTAGGGATCTCATCTGTGCGCAAAAGTTTGGAGGCT
CCGGAGGAATTATCCTCTTCCTGGCCCTTATCACACTTGCCACATGCGAGC
TGTACCACTATCAGGAATGCGTGCGAGGCACTACAGTGTCCGGTAGCTCTG
GATCACATCTGAGAATCGCTGGCCACCACTTGGGACGCTGCGATATCAAGG
ACCTACCTAAAGAGATCACCGTGGCTACCTCACGTACCCTCTCATACTACA
AACTGGGCGCTAGCCAGAGAGTTGCCGGGGACAGCGGAGGTGGATCGGGA
GGCCTACCCAATAATACCGCTTCATGGTTTACAGCACTGACACAGCATGGG
AAAGAGGACCTCAAATTCCCACGAGGCCAAGGGGTGCCAGGAGGGTCAGG
AGGTAAAGACTTCGGAGGGTTTAACTTTAGTCAGATTTTACCCGATCCTAG
TAAGCCAAGCAAACGCTCTTTCATCGAAGATCTGCTATTTAACAAGGTTAC
ACTGGCTGACGCTTCGGGATCAAGCGGGAGTAAGGAGGATCTTAAGTTTCC
TCGCGGACAGGGTGTGCCGATAAATACCAATAGCTCCCCAGACGATCAGA
TCGGATATTACAGGCGCGCGACGAGGAGAATTCGGGGCGGCTCGGGCTCT
AGCGGCTCACCATCTATTATCAGCAATGAGAAACAGGAGATTCTGGGAAC
AGTTTCTTGGAACTTGCGTGAAATGCTGGCTCATGGCGGATCTGGTGGGTA
A

### EMBODIMENTS

1. An immunogenic construct, the construct being:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i), preferably wherein said nucleotide sequence does not encode a targeting unit that targets antigen presenting cells and does encode a multimerization unit.

2. The immunogenic construct according to embodiment 1, the construct being:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77 and wherein the nucleotide sequence further encodes a targeting unit that targets antigen presenting cells and a multimerization unit; or
(ii) a polypeptide encoded by the nucleic acid sequence defined in (i); or
(iii) a multimeric protein consisting of multiple polypeptides as defined in (ii).

3. The immunogenic construct according to embodiment 2, wherein the multimerization unit is a dimerization unit and the multimeric protein is a dimeric protein consisting of two polypeptides as defined in (ii).

4. The immunogenic construct according to any of the preceding embodiments, wherein the antigenic unit comprises at least 77 SARS-CoV-2 T cell epitopes with amino acid sequences having at least 75% to 80% sequence identity to the amino acid sequences SEQ ID NOs: 1-77, preferably at least 81% to 85%, more preferably at least 86% to 90% or at least 91% to 95% and most preferably at least 96% to 99%.

5. The immunogenic construct according any of the preceding embodiments, wherein the 77 SARS-CoV-2 T cell epitopes have the amino acid sequences of SEQ ID NOs: 1-77.

6. The immunogenic construct according any of the preceding embodiments, wherein the antigenic unit further comprises one or more T cell epitopes with amino acid sequences having at least 73% sequence identity to the amino acid sequences SEQ ID NOs: 78-96, preferably all of said T cell epitopes.

7. The immunogenic construct according to embodiment 6, wherein the one or more T cell epitopes have amino acid sequences having at least 75% to 80% sequence identity to the amino acid sequences SEQ ID NOs: 78-96, preferably at least 81% to 85%, more preferably at least 86% to 90% or at least 91% to 95% and most preferably at least 96% to 99%.

8. The immunogenic construct according to embodiment 6, wherein the one or more T cell epitopes have amino acid sequences of SEQ ID NOs: 78-96.

9. The immunogenic construct according to embodiments 1 to 5, wherein the antigenic unit further comprises one or more T cell epitopes with amino acid sequences having at least 73% sequence identity to the amino acid sequences SEQ ID NOs: 97-160, preferably all of said T cell epitopes.

10. The immunogenic construct according to embodiment 9, wherein the one or more T cell epitopes have amino acid sequences having at least 75% to 80% sequence identity to the amino acid sequences SEQ ID NOs: 97-160, preferably at least 81% to 85%, more preferably at least 86% to 90% or at least 91% to 95% and most preferably at least 96% to 99%.

11. The immunogenic construct according to embodiment 9, wherein the one or more T cell epitopes have amino acid sequences of SEQ ID NOs: 97-160.

12. The immunogenic construct according to embodiments 6 to 8, wherein the antigenic unit further comprises one or more T cell epitopes with amino acid sequences having at least 73% sequence identity to the amino acid sequences SEQ ID NOs: 97-160, preferably all of said T cell epitopes.

13. The immunogenic construct according to embodiment 12, wherein the one or more T cell epitopes have amino acid sequences having at least 75% to 80% sequence identity to the amino acid sequences SEQ ID NOs: 97-160, preferably at least 81% to 85%, more preferably at least 86% to 90% or at least 91% to 95% and most preferably at least 96% to 99%.

14. The immunogenic construct according to embodiment 12, wherein the one or more T cell epitopes have amino acid sequences of SEQ ID NOs: 97-160.

15. The immunogenic construct according to any of the preceding embodiments, wherein some or all of the T cell epitopes are present as single, discrete epitopes.

16. The immunogenic construct according to embodiment 15, wherein one or more of the T cell epitopes are flanked by amino acids sequences also flanking said epitopes in the naturally occurring protein the epitope is derived from.

17. The immunogenic construct according to embodiment 16, wherein one or more of the T cell epitopes are flanked by amino acid sequences in the direction of the N-terminus and/or the C-terminus of the epitope.

18. The immunogenic construct according to any of embodiments 16 to 17, wherein such flanking sequences comprise 1 or more, 2 or more, 3 or more, 4 or more, 5 or more, 6 or more, 7 or more, 8 or more, 9 or more or 10 or more amino acids, preferably 1 to 10 amino acids, e.g. 2 to 8 amino acids or 3 to 7 amino acids or 4 to 5 amino acids.

19. The immunogenic construct according to any of embodiments 1 to 14, wherein the some or all of the T cell epitopes are present as groups of T cell epitopes, wherein each group comprises at least 2 T cell epitopes.

20. The immunogenic construct according to embodiment 19, wherein each group comprises from 2 to 20 T cell epitopes, preferably from 2 to 15 T cell epitopes, such as 2, 3, 4, 5, 6, 7, 8, 8, 10, 11, 12, 13, 14, or 15 T cell epitopes.

21. The immunogenic construct according to any of embodiments 19 to 20, wherein the at least 2 T cell epitopes are derived from the same SARS-CoV-2 protein, preferably from the same part of the same SARS-CoV-2 protein.

22. The immunogenic construct according to any of embodiments 19 to 21, wherein the T cell epitopes within a group are sequentially arranged, optionally separated by T cell epitope linkers.

23. The immunogenic construct according to any of embodiments 19 to 22, wherein the T cell epitopes within a group are aligned to form a continuous sequence of amino acids which corresponds to that of the naturally occurring protein the epitopes are derived from.

24. The immunogenic construct according to any of embodiments 19 to 23, wherein one or more of the groups of T cell epitopes are flanked by amino acids sequences also flanking the group in the naturally occurring protein the group of T cell epitopes is derived from.

25. The immunogenic construct according to embodiment 24, wherein one or more of the groups of T cell epitopes are flanked by amino acid sequences in the direction of the N-terminus and/or the C-terminus of the group of epitopes.

26. The immunogenic construct according to any of embodiments 24 to 25, wherein such flanking sequences comprise 1 or more, 2 or more, 3 or more, 4 or more, 5 or more, 6 or more, 7 or more, 8 or more, 9 or more or 10 or more amino acids, preferably 1 to 10 amino acids, e.g. 2 to 8 amino acids or 3 to 7 amino acids or 4 to 5 amino acids.

27. The immunogenic construct according to any of embodiments 24 to 26, wherein the resulting length of the group of epitopes plus flanking sequences is from about 18 amino acids to about 80 amino acids, such as from 18 amino acids to 80 amino acids.

28. The immunogenic construct according to any of embodiments 1 to 14, wherein some of the T cell epitopes are present as single, discrete epitopes and some others are present as groups of T cell epitopes, wherein each group comprises at least 2 T cell epitopes.

29. The immunogenic construct according to embodiment 28, wherein each group comprises from 2 to 20 T cell epitopes, preferably from 2 to 15 T cell epitopes, such as 2, 3, 4, 5, 6, 7, 8, 8, 10, 11, 12, 13, 14, or 15 T cell epitopes.

30. The immunogenic construct according to any of embodiments 28 to 29, wherein the at least 2 T cell epitopes are derived from the same SARS-CoV-2 protein, preferably from the same part of the same SARS-CoV-2 protein.

31. The immunogenic construct according to any of embodiments 27 to 30, wherein the T cell epitopes within a group are sequentially arranged.

32. The immunogenic construct according to any of embodiments 28 to 31, wherein the T cell epitopes within a group are aligned to form a continuous sequence of amino acids which corresponds to that of the naturally occurring protein the epitopes are derived from.

33. The immunogenic construct according to any of embodiments 28 to 32, wherein one or more of the T cell epitopes and/or one or more of the groups of T cell epitopes are flanked by amino acids sequences also flanking said epitopes or group of epitope in the naturally occurring protein the epitope or group is derived from.

34. The immunogenic construct according to embodiment 33, wherein one or more of the T cell epitopes and/or one or more of groups of T cell epitopes are flanked by amino acid sequences in the direction of the N-terminus and/or the C-terminus of the epitope/group of epitope.

35. The immunogenic construct according to any of embodiments 33 to 34, wherein such flanking sequences comprise 1 or more, 2 or more, 3 or more, 4 or more, 5 or more, 6 or more, 7 or more, 8 or more, 9 or more or 10 or more amino acids, preferably 1 to 10 amino acids, e.g. 2 to 8 amino acids or 3 to 7 amino acids or 4 to 5 amino acids.

36. The immunogenic construct according to any of embodiments 33 to 35, wherein the resulting length of the group of epitopes plus flanking sequences is from about 18 amino acids to about 80 amino acids, such as from 18 amino acids to 80 amino acids.

37. The immunogenic construct according to any of the preceding embodiments, wherein the following T cell epitopes are grouped together: the epitopes with SEQ ID NOs: 1-4, the epitopes with SEQID NOs: 5-9, the epitopes with SEQ ID NOs: 10-18, the epitopes with SEQ ID NOs: 19-20, the epitopes with SEQ ID NOs: 22-23, the epitopes with SEQ ID NOs: 24-26, the epitopes with SEQ ID NOs: 27-39, the epitopes with SEQ ID NOs: 40-43, the epitopes with SEQ ID NOs: 44-47, the epitopes with SEQ ID NOs: 48-58, the epitopes with SEQ ID NOs: 59-65, the epitopes with SEQ ID NOs: 66-71 and the epitopes with SEQ ID NOs: 72-77, or the aforementioned T cell epitopes with amino acid sequences having at least 73% sequence identity to the aforementioned SEQ ID NOs.

38. The immunogenic construct according to embodiment 37, wherein the group comprising the T cell epitopes with SEQ ID NOs: 5-9 is split up into two groups, with the first group comprising the T cell epitopes with SEQ ID NOs: 5-6 and the second group comprising the T cell epitopes with SEQ ID NOs: 7-9 or the aforementioned T cell epitopes with amino acid sequences having at least 73% sequence identity to the aforementioned SEQ ID NOs.

39. The immunogenic construct according to any of embodiments 37 to 38, wherein the group comprising the T cell epitopes with SEQ ID NOs: 10-18 is split up into two groups, with the first group comprising the T cell epitopes with SEQ ID NOs: 10-16 and the second group comprising the T cell epitopes with SEQ ID NOs: 17-18 or the aforementioned T cell epitopes with amino acid sequences having at least 73% sequence identity to the aforementioned SEQ ID NOs.

40. The immunogenic construct according to any of embodiments 37 to 39, wherein the group comprising the T cell epitopes with SEQ ID NOs: 72-77 is split up into two groups, with the first group comprising the T cell epitopes with SEQ ID NOs: 72-74 and the second group comprising the T cell epitopes with SEQ ID NOs: 75-77 or the aforementioned T cell epitopes with amino acid sequences having at least 73% sequence identity to the aforementioned SEQ ID NOs.

41. The immunogenic construct according to any of embodiments 37 to 40, wherein the antigenic unit further comprises one or more groups of T cell epitopes with SEQ ID NOs: 97-160 or amino acid sequences having at least 73% sequence identity to SEQ ID NOs: 97-160, wherein the following T cell epitopes are grouped together: the epitopes with SEQ ID NOs: 97-102, the epitopes with SEQ ID NOs: 103-109, the epitopes with SEQ ID NOs: 110-111, the epitopes with SEQ ID NOs: 112-113, the epitopes with SEQ ID NOs: 114-118, the epitopes with SEQ ID NOs: 120-123, the epitopes with SEQ ID NOs: 124-129, the epitopes with SEQ ID NOs: 130-134, the epitopes with SEQ ID NOs: 135-137, the epitopes with SEQ ID NOs: 138-140, the epitopes with SEQ ID NOs: 141-146, the epitopes with SEQ ID NOs: 147-152, the epitopes with SEQ ID NOs: 153-160, or the aforementioned T cell epitopes with amino acid sequences having at least 73% sequence identity to the aforementioned SEQ ID NOs.

42. The immunogenic construct according to embodiment 41, wherein the group comprising the T cell epitopes with SEQ ID NOs: 114-118 is split up into two groups, with the first group comprising the T cell epitopes with SEQ ID NOs: 114-115 and the second group comprising the T cell epitopes with SEQ ID NOs: 116-118, or the aforementioned T cell epitopes with amino acid sequences having at least 73% sequence identity to the aforementioned SEQ ID NOs.

43. The immunogenic construct according to any of embodiments 41 to 42, wherein the group comprising the T cell epitopes with SEQ ID NOs: 141-146 is split up into two groups, with the first group comprising the T cell epitopes with SEQ ID NOs: 141-143 and the second group comprising the T cell epitopes with SEQ ID NOs: 144-146, or the aforementioned T cell epitopes with amino acid sequences having at least 73% sequence identity to the aforementioned SEQ ID NOs.

44. The immunogenic construct according to any of embodiments 41 to 43, wherein the group comprising the T cell epitopes with SEQ ID NOs: 147-152 is split up into two groups, with the first group comprising the T cell epitopes with SEQ ID NOs: 147-149 and the second group comprising the T cell epitopes with SEQ ID NOs: 150-152, or the aforementioned T cell epitopes with amino acid sequences having at least 73% sequence identity to the aforementioned SEQ ID NOs.

45. The immunogenic construct according to any of embodiments 41 to 44, wherein the group comprising the T cell epitopes with SEQ ID NOs: 153-160 is split up into two groups, with the first group comprising the T cell epitopes with SEQ ID NOs: 153-157 and the second group comprising the T cell epitopes with SEQ ID NOs: 158-160, or the aforementioned T cell epitopes with amino acid sequences having at least 73% sequence identity to the aforementioned SEQ ID NOs.

46. The immunogenic construct according to any of embodiments 37 to 45, wherein the antigenic unit further comprises one or more groups of T cell epitopes with SEQ ID NOs: 78-96 or amino acid sequences having at least 73% sequence identity to SEQ ID NOs: 78-96, wherein the following T cell epitopes are grouped together: the epitopes with SEQ ID NOs: 78-84 and the epitopes with SEQ ID NOs: 86-96, or the aforementioned T cell epitopes with amino acid sequences having at least 73% sequence identity to the aforementioned SEQ ID NOs.

47. The immunogenic construct according to embodiment 46, wherein the group comprising the T cell epitopes with SEQ ID NOs: 78-84 is split up into two groups, with the first group comprising the T cell epitopes with SEQ ID NOs: 78-79 and the second group comprising the T cell epitopes with SEQ ID NOs: 80-84, or the aforementioned T cell epitopes with amino acid sequences having at least 73% sequence identity to the aforementioned SEQ ID NOs.

48. The immunogenic construct according to any of embodiments 46 to 47, wherein the group comprising the T cell epitopes with SEQ ID NOs: 86-96 is split up into two groups, with the first group comprising the T cell epitopes with SEQ ID NOs: 86-93 and the second group comprising the T cell epitopes with SEQ ID NOs: 94-96, or the aforementioned T cell epitopes with amino acid sequences having at least 73% sequence identity to the aforementioned SEQ ID NOs.

49. The immunogenic construct according to any of embodiments 37 to 48, wherein the antigenic unit further comprises the T cell epitope with the SEQ ID NO: 21, or with an amino acid sequence having at least 73% sequence identity to SEQ ID NO: 21, as a single, discrete epitope.

50. The immunogenic construct according to embodiment 49, wherein the antigenic unit further comprises one or more of the following T cell epitopes as a single, discrete epitope: the T cell epitope with the SEQ ID NO: 85 and the T cell epitope with the SEQ ID NO: 119, or the aforementioned T cell epitopes with amino acid sequences having at least 73% sequence identity to SEQ ID NOs: 85 and 119, respectively.

51. The immunogenic construct according to any of the preceding embodiments, wherein some or all of the T cell epitopes and/or some or all of the groups of T cell epitopes are separated from each other by T cell epitope linkers.

52. The immunogenic construct according to embodiment 51, wherein the T cell epitope linkers are non-immunogenic and preferably flexible.

53. The immunogenic construct according to any of embodiments 51 to 52, wherein all T cell epitope linkers comprised in the antigenic unit are identical.

54. The immunogenic construct according to any of embodiments 51 to 53, wherein the T cell epitope linkers are serine and/or glycine-rich linkers.

55. The immunogenic construct according to any of embodiments 51 to 54, wherein the T cell epitope linkers are serine and/or glycine-rich linkers which further comprise at least one leucine residue.

56. The immunogenic construct according to any of the preceding embodiments, wherein the antigenic unit comprises an amino acid sequence having at least 73% sequence identity to an amino acid sequence selected from the group consisting of SEQ ID NOs: 161, 162, 163, 164, 165, 166 and 167.

57. The immunogenic construct according to embodiment 56, wherein the antigenic unit comprises an amino acid sequence having at least 75% to 80% sequence identity to an amino acid sequence selected from the group consisting of SEQ ID NOs: 161, 162, 163, 164, 165, 166 and 167, preferably at least 81% to 85%, more preferably at least 86% to 90% or at least 91% to 95% and most preferably at least 96% to 99%.

58. The immunogenic construct according to embodiment 56, wherein the antigenic unit comprises an amino acid sequence selected from the group consisting of SEQ ID NOs: 161, 162, 163, 164, 165, 166 and 167, preferably wherein the antigenic unit has an amino acid sequence selected from the group consisting of SEQ ID NOs: 161, 162, 163, 164, 165, 166 and 167, more preferably wherein the antigenic unit comprises the amino acid sequence of SEQ ID NO: 167 or even more preferably wherein the antigenic unit has the amino acid sequence of SEQ ID NO: 167.

59. The immunogenic construct according to any of the previous embodiments, wherein the targeting unit is or comprises a moiety that interacts with surface molecules on the antigen presenting cells, preferably wherein the targeting unit is or comprises a moiety that interacts with surface molecules on human antigen-presenting cells.

60. The immunogenic construct according to embodiment 59, wherein the surface molecule is selected from the group consisting of MHC, CD14, CD40, CLEC9A, chemokine receptors, such as CCR1, CCR3, CCR4, CCR5, CCR6, CCR7, CCR8 and XCR1 and Toll-like receptors such as TLR-2, TLR-4 or TLR-5, preferably wherein the surface molecule is selected from the group consisting of HLA; hCD14, hCD40, hCLEC9A, human chemokine receptors, such as hCCR1, hCCR3, hCCR4, hCCR5, hCCR6, hCCR7, hCCR8 and hXCR1 and Toll-like receptors such as hTLR-2, hTLR-4 or hTLR-5.

61. The immunogenic construct according to any of embodiments 59 to 60, wherein the targeting unit comprises or consists of soluble CD40 ligand, preferably human soluble CD40 ligand, CCL4 and its isoforms, preferably human CCL4 and its isoforms, CCL5, preferably human CCL5, CCL19, preferably human CCL19, CCL20, preferably human CCL20, CCL21, preferably human CCL21, macrophage inflammatory protein alpha including its isoforms, such as mouse CCL3, human CCL3, human CCL3L1, human CCL3L2 and human CCL3L3, XCL1, preferably human XCL1, XCL2, preferably human XCL2, flagellin, anti-HLA-DP, anti-HLA-DR, anti-pan HLA class II, anti-CD40, preferably anti-human CD40, anti-TLR-2, preferably anti-human TLR-2, anti-TLR-4, preferably anti-human TLR-4, anti-TLR-5, preferably anti-human TLR-5 or anti-CLEC9A, preferably anti-human CLEC9A.

62. The immunogenic construct according to embodiment 61, wherein the targeting unit comprises or consists of human MIP-1α (LD780, CCL3L1).

63. The immunogenic construct according to embodiment 62, wherein the targeting unit comprises an amino acid sequence having at least 80% sequence identity to the amino acid sequence 24-93 of SEQ ID NO: 168, such as comprising the amino acid sequence 26-93 of SEQ ID NO: 168 or comprising the amino acid sequence 28-93 of SEQ ID NO: 168.

64. The immunogenic construct according to embodiment 63, wherein the targeting unit consists of an amino acid sequence having at least 80% sequence identity to the amino acid sequence 24-93 of SEQ ID NO: 168, such as consisting of the amino acid sequence 26-93 of SEQ ID NO: 168 or consisting of the amino acid sequence 28-93 of SEQ ID NO: 168.

65. The immunogenic construct according to embodiment 64, wherein the targeting unit consists of the amino acid sequence 24-93 of SEQ ID NO: 168 or consists of the amino acid sequence 26-93 of SEQ ID NO: 168.

66. The immunogenic construct according to embodiment 61, wherein the targeting unit comprises an amino acid sequence having at least 80% sequence identity to the amino acid sequence 20-260 of SEQ ID NO: 169, such as comprising the amino acid sequence 20-260 of SEQ ID NO: 169.

67. The immunogenic construct according to embodiment 66, wherein the targeting unit consists of an amino acid sequence having at least 80% sequence identity to the amino acid sequence 20-260 of SEQ ID NO: 169, such as consists of the amino acid sequence 20-260 of SEQ ID NO: 169.

68. The immunogenic construct according to embodiment 67, wherein the targeting unit consists of the amino acid sequence 20-260 of SEQ ID NO: 169.

69. The immunogenic construct according to any of embodiments 2 to 68, wherein the multimerization unit is selected from the group consisting of dimerization unit, trimerization unit and tetramerization unit and wherein said multimerization unit optionally comprises a hinge region which has the ability to form one or more covalent bonds.

70. The immunogenic construct according to embodiment 69, wherein the multimerization unit is a trimerization unit, such as a human collagen-derived trimerization or the C-terminal domain of T4 fibritin.

71. The immunogenic construct according to embodiment 70, wherein the multimerization unit is a human collagen-derived trimerization unit, preferably one selected from the group consisting of human collagen derived XVIII-derived trimerization domain and human collagen XV-derived trimerization domain.

72. The immunogenic construct according to embodiment 69, wherein the multimerization unit is a tetramerization unit which is a domain derived from p53.

73. The immunogenic construct according to any of embodiments 69 to 72, wherein the multimerization unit comprises a hinge region which has the ability to form one or more covalent bonds.

74. The immunogenic construct according to any of embodiments 69 to 73, wherein the hinge region is Ig derived, such as derived from human Ig, such as derived from hIgG1 or hIgG2 or hIgG3 or from hIgM.

75. The immunogenic construct according to any of embodiments 69 and 73 to 74, wherein the multimerization unit is a dimerization unit and said dimerization unit further comprises another domain that facilitates dimerization.

76. The immunogenic construct according to embodiment 75, wherein the other domain is an immunoglobulin domain, preferably an immunoglobulin constant domain.

77. The immunogenic construct according to any of embodiments 75 to 76, wherein the other domain is a carboxyterminal C domain derived from IgG, preferably from IgG3, more preferably from hIgG3.

78. The immunogenic construct according to any of embodiments 75 to 77 wherein the dimerization unit further comprises a dimerization unit linker, such as glycine-serine rich linker, such as GGGSSGGGSG.

79. The immunogenic construct according to embodiment 78, wherein the dimerization unit linker connects the hinge region and the other domain that facilitates dimerization.

80. The immunogenic construct according to any of embodiments 75 to 79, wherein the dimerization unit comprises hinge exon h1 and hinge exon h4, a dimerization unit linker and a CH3 domain of human IgG3.

81. The immunogenic construct according to embodiment 80, wherein the dimerization unit comprises an amino acid sequence having at least 80% sequence identity to the amino acid sequence 94-237 of SEQ ID NO: 168.

82. The immunogenic construct according to embodiment 81, wherein the dimerization unit consists of an amino acid sequence having at least 80% sequence identity to the amino acid sequence 94-237 of SEQ ID NO: 168.

83. The immunogenic construct according to embodiment 82, wherein the dimerization unit consists of the amino acid sequence 94-237 of SEQ ID NO: 168.

84. The immunogenic construct according to any of the preceding embodiments, wherein the construct is the polynucleotide (i), preferably wherein the polynucleotide comprises a nucleotide sequence which further encodes a signal peptide.

85. The immunogenic construct according to embodiment 84, wherein the signal peptide is the natural leader sequence of the targeting unit.

86. The immunogenic construct according to embodiment 85, wherein the signal peptide is an Ig VH signal peptide, a human TPA signal peptide or a human MIP1-α (CCL3L1) signal peptide, preferably wherein the polynucleotide comprises a nucleotide sequence which encodes a targeting unit that comprises or consists of human MIP-1α (LD780, CCL3L1) and the signal peptide is a human MIP1-α (CCL3L1) signal peptide.

87. The immunogenic construct according to any of embodiments 84 to 86, wherein the polynucleotide comprises a nucleotide sequence which encodes a targeting unit comprising an amino acid sequence having at least 80% sequence identity to the amino acid sequence 24-93 of SEQ ID NO: 168, such as comprising the amino acid sequence 26-93 of SEQ ID NO: 168 or comprising the amino acid sequence 28-93 of SEQ ID NO: 168 and further encodes a signal peptide that comprises an amino acid sequence having at least 85% sequence identity to the amino acid sequence 1-23 of SEQ ID NO: 168.

88. The immunogenic construct according to any of the preceding embodiments, wherein the construct is the polynucleotide.

89. The immunogenic construct according to embodiment 88, wherein the polynucleotide is the nucleotide sequence as defined in any of embodiments 1 to 87.

90. The immunogenic construct according to any of embodiments 88 to 89, wherein the polynucleotide is a DNA or an RNA.

91. A polynucleotide as defined in any of the embodiments 1 to 90, 124, 125 and 127.

92. A vector comprising the polynucleotide according to embodiment 91.

93. The vector according to embodiment 92, wherein the vector is a polycistronic vector which comprises: a) the polynucleotide according to embodiment 91 and b) one or more nucleic acid sequences encoding one or more immunostimulatory compounds, wherein the vector allows for the co-expression of a polypeptide encoded by the polynucleotide and the one or more immunostimulatory compounds as separate molecules.

94. The vector according to embodiment 93, wherein vector comprises one or more co-expression elements, such as co-expression elements selected from the group consisting of IRES elements, 2A peptides, promoters and bidirectional promoters.

95. The vector according to any of embodiments 92 or 93, wherein the one or more immunostimulatory compound is a compound that stimulates APCs and the stimulation results in attraction, activation, maturation and/or proliferation of APCs.

96. The vector according to any of embodiments 92 to 94, wherein the vector is selected from the group consisting of DNA vector and RNA vector.

97. The vector according to embodiment 96, wherein the vector is a DNA vector, such as a DNA plasmid or DNA viral vector, such as a DNA viral vector selected from the group consisting of adenovirus, vaccinia virus, adeno-associated virus, cytomegalovirus and Sendai virus.

98. The vector according to embodiment 96, wherein the vector is an RNA vector, such as an RNA plasmid or RNA viral vector, such as a retroviral vector, such as a retroviral vector selected from the group consisting of alphavirus, lentivirus, Moloney murine leukemia virus and rhabdovirus.

99. A method of producing a vector as defined in any of embodiments 92 to 98, the method comprising:
a) transfecting or transducing cells in vitro with the vector;
b) culturing said cells;
c) optionally lysing the cells to release the vector from the cells; and
d) isolating and optionally purifying the vector.

100. A host cell comprising the polynucleotide according to embodiment 91 or the vector according to any of embodiments 92 to 98.

101. A polypeptide encoded by the nucleic acid as defined in any of the embodiments 1 to 83 and as defined in embodiments 123 and 126.

102. A multimeric protein consisting of multiple polypeptides according to embodiment 101.

103. The multimeric protein according to embodiment 102, which is a dimeric protein consisting of two polypeptides as defined in claim 101, such as a homodimeric protein or heterodimeric protein, preferably a homodimeric protein.

104. A method for preparing the polypeptide according to embodiment 101, the method comprises:
a) transfecting or transducing cells with a polynucleotide according to claim 91 or a vector comprising such polynucleotide;
b) culturing the cells;
c) isolating the polypeptide from the cells; and
d) optionally purifying the isolated polypeptide.

105. A method for preparing the multimeric protein according to any of embodiments 102 to 103, the method comprises:
a) transfecting or transducing cells with a polynucleotide according to claim 91 or a vector comprising such polynucleotide;
b) culturing the cells;
c) isolating the multimeric protein from the cells; and
d) optionally purifying the isolated multimeric protein.

106. The immunogenic construct according to any of embodiments 1 to 90 and 123 to 127 or the polynucleotide according to embodiment 91 or the vector according to any of embodiments 92 to 98 or the polypeptide according to embodiment 101 or the multimeric protein according to any of embodiments 102 to 103 for use as a medicament.

107. A pharmaceutical composition or vaccine comprising the immunogenic construct according to any of embodiments 1 to 90 and 123 to 127 or the polynucleotide according to embodiment 91 or the vector according to any of embodiments 92 to 98 or the polypeptide according to embodiment 101 or the multimeric protein according to any of embodiments 102 to 103 and a pharmaceutically acceptable carrier.

108. The pharmaceutical composition or vaccine according to embodiment 107, comprising a pharmaceutically acceptable carrier and the vector according to any of embodiments 92 to 98.

109. The pharmaceutical composition or vaccine according to any of embodiments 107 to 108, wherein the pharmaceutically acceptable carrier is selected from the group consisting of saline, buffered saline, such as PBS, dextrose, water, glycerol, ethanol, aqueous buffers, such as isotonic aqueous buffers or Tyrode's buffer, and combinations thereof.

110. The pharmaceutical composition or vaccine according to any of embodiments 107 to 109, wherein the composition or vaccine further comprises molecules that facilitate the transfection of cells with the polynucleotide or vector.

111. The pharmaceutical composition or vaccine according to any of embodiments 107 to 110, wherein the composition or vaccine further comprises a pharmaceutically acceptable amphiphilic block co-polymer comprising blocks of poly(ethylene oxide) and polypropylene oxide, such as comprising said pharmaceutically acceptable amphiphilic block co-polymer in an amount of from 0.2% w/v to 20% w/v.

112. The pharmaceutical composition or vaccine according to any of embodiments 107 and 109 to 110, wherein the composition or vaccine comprises the polypeptide or the multimeric protein in a range of from 5 μg to 5 mg.

113. The pharmaceutical composition according to any of embodiments 107 to 111, wherein the composition or vaccine comprises the polynucleotide or vector in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg.

114. The pharmaceutical composition or vaccine according to any of embodiments 107 to 113 for use in the treatment of a disease caused by SARS-CoV-2.

115. The pharmaceutical composition or vaccine according to embodiment 114, wherein the treatment is a prophylactic treatment.

116. The pharmaceutical composition or vaccine according to embodiment 114, wherein the treatment is a therapeutic treatment.

117. A method for treating a disease caused by SARS-CoV-2, said method comprises administering to a subject the pharmaceutical composition or vaccine according to any of embodiments 107 to 113.

118. The method according to embodiment 117, wherein said subject is in need of prevention of such disease and said method is a prophylactic treatment.

119. The method according to embodiment 117, wherein said subject has such disease and said method is a therapeutic treatment.

120. A method for treating a disease caused by SARS-CoV-2, said method comprises administering to a subject who has been previously vaccinated with a SARS-CoV-2 vaccine the pharmaceutical composition or vaccine according to any of embodiments 107 to 113.

121. The method according to embodiment 120, wherein said method is a prophylactic treatment.

122. The method according to embodiment 120, wherein said method is a therapeutic treatment.

123. The immunogenic construct according to any of embodiments 1 to 90, the construct being a polypeptide that has an amino acid sequence selected from the group consisting of SEQ ID NOs: 171, 172, 173, 174, 175, 176 and 177 or a dimeric protein consisting of two such polypeptides, preferably a polypeptide that has the amino acid of SEQ ID NO: 177 or a dimeric protein consisting of two such polypeptides.

124. The immunogenic construct according to any of embodiments 1 to 90, the construct being a polynucleotide comprising a nucleotide sequence encoding a polypeptide that has an amino acid sequence selected from the group consisting of SEQ ID NOs: 178, 179, 180, 181, 182, 183 and 184, preferably comprising a nucleotide sequence encoding a polypeptide that has the amino acid sequence of SEQ ID NO: 184.

125. The immunogenic construct according to any of embodiments 1 to 90, the construct being a polynucleotide comprising the nucleotide sequence of SEQ ID NO: 185.

126: The immunogenic construct according to any of embodiments 1 to 90, the construct being a polypeptide that has an amino acid sequence selected from the group consisting of SEQ ID NOs: 161, 162, 163, 164, 165, 166 and 167.

127. The immunogenic construct according to any of embodiments 1 to 90, the construct being a polynucleotide comprising a nucleotide sequence encoding a polypeptide that has an amino acid sequence selected from the group consisting of SEQ ID NOs: 161, 162, 163, 164, 165, 166 and 167.

128. A pharmaceutical composition/vaccine comprising the immunogenic construct according to any of embodiments 123 to 127 and a pharmaceutically acceptable carrier.

129. The pharmaceutical composition/vaccine according to embodiment 128, comprising a vector comprising the immunogenic construct of embodiment 125 and a pharmaceutically acceptable carrier.

1. An immunogenic construct, the construct being:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77; or
(ii) a polypeptide encoded by the nucleic acid sequence defined in (i).

2. The immunogenic construct according to claim 1, wherein the nucleotide sequence does not encode a targeting unit that targets antigen presenting cells and does not encode a multimerization unit.

3. The immunogenic construct according to claim 1, the construct being:
(i) a polynucleotide comprising a nucleotide sequence encoding an antigenic unit, wherein the antigenic unit comprises at least 77 SARS-CoV-2 T cell epitopes with amino acid sequences having at least 73% sequence identity to the amino acid sequences SEQ ID NOs: 1-77 and wherein the nucleotide sequence further encodes a targeting unit that targets antigen presenting cells and a multimerization unit; or
(ii) a polypeptide encoded by the nucleic acid sequence defined in (i); or
(iii) a multimeric protein consisting of multiple polypeptides as defined in (ii).

4. The immunogenic construct according any of the preceding claims, wherein the 77 SARS-CoV-2 T cell epitopes have the amino acid sequences of SEQ ID NOs: 1-77.

5. The immunogenic construct according any of the preceding claims, wherein the antigenic unit further comprises one or more T cell epitopes with amino acid sequences having at least 73% sequence identity to the amino acid sequences SEQ ID NOs: 78-96, preferably wherein the antigenic unit further comprises all of said T cell epitopes.

6. The immunogenic construct according to claim 5, wherein the one or more T cell epitopes have amino acid sequences of SEQ ID NOs: 78-96.

7. The immunogenic construct according to claims 1 to 4, wherein the antigenic unit further comprises one or more T cell epitopes with amino acid sequences having at least 73% sequence identity to the amino acid sequences SEQ ID NOs: 97-160, preferably wherein the antigenic unit further comprises all of said T cell epitopes.

8. The immunogenic construct according to claim 7, wherein the one or more T cell epitopes have amino acid sequences of SEQ ID NOs: 97-160.

9. The immunogenic construct according to claims 7 to 8, wherein the antigenic unit further comprises one or more T cell epitopes with amino acid sequences having at least 73% sequence identity to the amino acid sequences SEQ ID NOs: 97-160, preferably all of said the T cell epitopes.

10. The immunogenic construct according to claim 9, wherein the one or more T cell epitopes have amino acid sequences of SEQ ID NOs: 97-160.

11. The immunogenic construct according to any of the preceding claims, wherein some or all of the T cell epitopes are present as single, discrete epitopes and/or wherein some or all of the T cell epitopes are present as groups of T cell epitopes, wherein each group comprises at least 2 T cell epitopes.

12. The immunogenic construct according to any of the preceding claims, wherein some or all of the T cell epitopes are present as groups of T cell epitopes, wherein each group comprises at least 2 T cell epitopes.

13. The immunogenic construct according to claim 12, wherein the at least 2 T cell epitopes are derived from the same SARS-CoV-2 protein, preferably from the same part of the same SARS-CoV-2 protein.

14. The immunogenic construct according to any of claims 11 to 13, wherein the T cell epitopes within a group are sequentially arranged, optionally separated by a T cell epitope linker.

15. The immunogenic construct according to any of claims 11 to 13, wherein the T cell epitopes within a group are aligned to form a continuous sequence of amino acids which corresponds to that of the naturally occurring protein the epitopes are derived from.

16. The immunogenic construct according to any of the previous claims, wherein one or more of the T cell epitopes are flanked by amino acids sequences also flanking said epitopes in the naturally occurring protein the epitope is derived from.

17. The immunogenic construct according to claim 16, wherein one or more of the T cell epitopes are flanked by amino acid sequences in the direction of the N-terminus and/or the C-terminus of the epitope.

18. The immunogenic construct according to any of claims 16 to 17, wherein such flanking sequences comprise 1 or more, 2 or more, 3 or more, 4 or more, 5 or more, 6 or more, 7 or more, 8 or more, 9 or more or 10 or more amino acids, preferably 1 to 10 amino acids, e.g. 2 to 8 amino acids or 3 to 7 amino acids or 4 to 5 amino acids.

19. The immunogenic construct according to any of the preceding claims, wherein some or all of the T cell epitopes and/or some or all of the groups of T cell epitopes are separated from each other by T cell epitope linkers, preferably non-immunogenic T cell epitope linkers which are preferably flexible linkers.

20. The immunogenic construct according to any of the preceding claims, wherein the antigenic unit comprises an amino acid sequence having at least 73% sequence identity to an amino acid sequence selected from the group consisting of SEQ ID NOs: 161, 162, 163, 164, 165, 166 and 167, such as at least 75% to 80% sequence identity to an amino acid sequence selected from the group consisting of SEQ ID NOs: 161, 162, 163, 164, 165, 166 and 167, preferably at least 81% to 85%, more preferably at least 86% to 90% or at least 91% to 95% and most preferably at least 96% to 99%.

21. The immunogenic construct according to any of the preceding claims, wherein the antigenic unit comprises an amino acid sequence selected from the group consisting of SEQ ID NOs: 161, 162, 163, 164, 165, 166 and 167, preferably wherein the antigenic unit has an amino acid sequence selected from the group consisting of SEQ ID NOs: 161, 162, 163, 164, 165, 166 and 167, more preferably wherein the antigenic unit comprises the amino acid sequence of SEQ ID NO: 167 or even more preferably wherein the antigenic unit has the amino acid sequence of SEQ ID NO: 167.

22. The immunogenic construct according to any of the preceding claims, wherein the targeting unit is or comprises a moiety that interacts with surface molecules on the antigen presenting cells, preferably wherein the targeting unit is or comprises a moiety that interacts with surface molecules on human antigen presenting cells.

23. The immunogenic construct according to claim 22, wherein the surface molecule is selected from the group consisting of MHC, CD14, CD40, CLEC9A, chemokine receptors, such as CCR1, CCR3, CCR4, CCR5, CCR6, CCR7, CCR8 and XCR1 and Toll-like receptors such as TLR-2, TLR-4 or TLR-5, preferably wherein the surface molecule is selected from the group consisting of HLA; hCD14, hCD40, hCLEC9A, human chemokine receptors, such as hCCR1, hCCR3, hCCR4, hCCR5, hCCR6, hCCR7, hCCR8 and hXCR1 and Toll-like receptors such as hTLR-2, hTLR-4 or hTLR-5.

24. The immunogenic construct according to any of the preceding claims, wherein the targeting unit comprises or consists of soluble CD40 ligand, preferably human soluble CD40 ligand, CCL4 and its isoforms, preferably human CCL4 and its isoforms, CCL5, preferably human CCL5, CCL19, preferably human CCL19, CCL20, preferably human CCL20, CCL21, preferably human CCL21, macrophage inflammatory protein alpha including its isoforms, such as mouse CCL3, human CCL3, human CCL3L1, human CCL3L2 and human CCL3L3, XCL1, preferably human XCL1, XCL2, preferably human XCL2, flagellin, anti-HLA-DP, anti-HLA-DR, anti-pan HLA class II, anti-CD40, preferably anti-human CD40, anti-TLR-2, preferably anti-human TLR-2, anti-TLR-4, preferably anti-human TLR-4, anti-TLR-5, preferably anti-human TLR-5 or anti-CLEC9A, preferably anti-human CLEC9A.

25. The immunogenic construct according to any of the preceding claims, wherein the targeting unit comprises or consists of human MIP-1α (LD780, CCL3L1).

26. The immunogenic construct according to any of the preceding claims, wherein the multimerization unit is selected from the group consisting of dimerization unit, trimerization unit and tetramerization unit and wherein said multimerization unit optionally comprises a hinge region which has the ability to form one or more covalent bonds.

27. The immunogenic construct according to claim 26, wherein the multimerization unit is a trimerization unit, such as a human collagen-derived trimerization such as a human collagen-derived trimerization unit selected from the group consisting of human collagen derived XVIII-derived trimerization domain and human collagen XV-derived trimerization domain, or the C-terminal domain of T4 fibritin.

28. The immunogenic construct according to claim 26, wherein the multimerization unit is a tetramerization unit which is a domain derived from p53.

29. The immunogenic construct according to any of the preceding claims, wherein the multimerization unit comprises a hinge region which has the ability to form one or more covalent bonds, preferably an Ig derived hinge region, such as derived from human Ig, such as derived from hIgG1 or hIgG2 or hIgG3 or from hIgM.

30. The immunogenic construct according to any of claims 26 and 29, wherein the multimerization unit is a dimerization unit and said dimerization unit further comprises another domain that facilitates dimerization.

31. The immunogenic construct according to claim 30, wherein the other domain is an immunoglobulin domain, preferably an immunoglobulin constant domain, more preferably a carboxyterminal C domain derived from IgG, preferably from IgG3, more preferably from hIgG3.

32. The immunogenic construct according to any of claims 30 to 31 wherein the dimerization unit further comprises a dimerization unit linker, such as glycine-serine rich linker, such as GGGSSGGGSG, preferably wherein the dimerization unit linker connects the hinge region and the other domain that facilitates dimerization.

33. The immunogenic construct according to any of claims 30 to 32, wherein the dimerization unit comprises hinge exon h1 and hinge exon h4, a dimerization unit linker and a CH3 domain of human IgG3.

34. The immunogenic construct according to any of the preceding claims, wherein the construct is the polynucleotide (i).

35. The immunogenic construct according to claim 34, wherein the polynucleotide comprises a nucleotide sequence which further encodes a signal peptide.

36. The immunogenic construct according to claim 35, wherein the signal peptide is the natural leader sequence of the targeting unit.

37. The immunogenic construct according to any of claims 34 to 36, wherein the polynucleotide is a DNA or an RNA.

38. The immunogenic construct according to any of the preceding claims, the construct being a polypeptide that has an amino acid sequence selected from the group consisting of SEQ ID NOs: 171, 172, 173, 174, 175, 176 and 177 or a dimeric protein consisting of two such polypeptides, preferably a polypeptide that has the amino acid of SEQ ID NO: 177 or a dimeric protein consisting of two such polypeptides.

39. The immunogenic construct according to any of claims 1 to 37, the construct being a polynucleotide comprising a nucleotide sequence encoding a polypeptide that has an amino acid sequence selected from the group consisting of SEQ ID NOs: 178, 179, 180, 181, 182, 183 and 184, preferably comprising a nucleotide sequence encoding a polypeptide that has the amino acid sequence of SEQ ID NO: 184.

40. The immunogenic construct according to any of claims 1 to 37, the construct being a polynucleotide comprising the nucleotide sequence of SEQ ID NO: 185.

41: The immunogenic construct according to any of claims 1 to 37, the construct being a polypeptide that has an amino acid sequence selected from the group consisting of SEQ ID NOs: 161, 162, 163, 164, 165, 166 and 167.

42. The immunogenic construct according to any of claims 1 to 37, the construct being a polynucleotide comprising a nucleotide sequence encoding a polypeptide that has an amino acid sequence selected from the group consisting of SEQ ID NOs: 161, 162, 163, 164, 165, 166 and 167.

43. A polynucleotide as defined in any of the preceding claims.

44. A vector comprising the polynucleotide according to claim 43.

45. The vector according to claim 44, wherein the vector is a polycistronic vector which comprises: a) the polynucleotide according to claim 43 and b) one or more nucleic acid sequences encoding one or more immunostimulatory compounds, wherein the vector allows for the co-expression of a polypeptide encoded by the polynucleotide and the one or more immunostimulatory compounds as separate molecules.

46. The vector according to any of claims 44 to 45, wherein the vector is selected from the group consisting of DNA vector and RNA vector, preferably selected from the group consisting of DNA plasmid and DNA viral vector, such as a DNA viral vector selected from the group consisting of adenovirus, vaccinia virus, adeno-associated virus, cytomegalovirus and Sendai virus or preferably selected from the group consisting of RNA plasmid and RNA viral vector, such as a retroviral vector selected from the group consisting of alphavirus, lentivirus, Moloney murine leukemia virus and rhabdovirus

47. A method of producing a vector as defined in any of claims 44 to 46, the method comprising:
a) transfecting or transducing cells in vitro with the vector;
b) culturing said cells;
c) optionally lysing the cells to release the vector from the cells; and
d) isolating and optionally purifying the vector.

48. A host cell comprising the polynucleotide according to claim 43 or the vector according to any of claims 44 to 46.

49. A polypeptide encoded by the nucleic acid as defined in any of claims 1 to 42.

50. A multimeric protein consisting of multiple polypeptides according to claim 49.

51. The multimeric protein according to claim 50, which is a dimeric protein consisting of two polypeptides as defined in claim 49.

52. A method for preparing the polypeptide according to claim 49, the method comprises:
a) transfecting or transducing cells with a polynucleotide according to claim 43 or a vector comprising such polynucleotide;
b) culturing the cells;
c) isolating the polypeptide from the cells; and
d) optionally purifying the isolated polypeptide.

53. A method for preparing the multimeric protein according to any of claims 50 to 51, the method comprises:
a) transfecting or transducing cells with a polynucleotide according to claim 43 or a vector comprising such polynucleotide;
b) culturing the cells;
c) isolating the multimeric protein from the cells; and
d) optionally purifying the isolated multimeric protein.

54. The immunogenic construct according to any of claims 1 to 42 or the polynucleotide according to claim 43 or the vector according to any of claims 43 to 46 or the polypeptide according to claim 49 or the multimeric protein according to any of claims 50 to 51 for use as a medicament.

55. A pharmaceutical composition or vaccine comprising the immunogenic construct according to any of claims 1 to 42 or the polynucleotide according to claim 43 or the vector according to any of claims 43 to 46 or the polypeptide according to claim 49 or the multimeric protein according to any of claims 50 to 51 and a pharmaceutically acceptable carrier.

56. The pharmaceutical composition or vaccine according to claim 55 for use in the treatment of a disease caused by SARS-CoV-2, wherein said treatment is a prophylactic treatment or a therapeutic treatment.

57. A method for treating a disease caused by SARS-CoV-2, said method comprises administering to a subject who has been previously vaccinated with a SARS-CoV-2 vaccine the pharmaceutical composition or vaccine according to claim 50, wherein said method is a prophylactic treatment or a therapeutic treatment.


SEQUENCE LISTING (288 sequences)

SEQ ID NO: 1 - protein, 19 aa, SARS-CoV-2
SRTLSYYKLGASQRVAGDS

SEQ ID NO: 2 - protein, 19 aa, SARS-CoV-2
PKEITVATSRTLSYYKLGA

SEQ ID NO: 3 - protein, 19 aa, SARS-CoV-2
LRIAGHHLGRCDIKDLPKE

SEQ ID NO: 4 - protein, 19 aa, SARS-CoV-2
GRCDIKDLPKEITVATSRT

SEQ ID NO: 5 - protein, 19 aa, SARS-CoV-2
TALTQHGKEDLKFPRGQGV

SEQ ID NO: 6 - protein, 19 aa, SARS-CoV-2
PNNTASWFTALTQHGKEDL

SEQ ID NO: 7 - protein, 19 aa, SARS-CoV-2
SSPDDQIGYYRRATRRIRG

SEQ ID NO: 8 - protein, 19 aa, SARS-CoV-2
QGVPINTNSSPDDQIGYYR

SEQ ID NO: 9 - protein, 19 aa, SARS-CoV-2
EDLKFPRGQGVPINTNSSP

SEQ ID NO: 10 - protein, 19 aa, SARS-CoV-2
VTPSGTWLTYTGAIKLDDK

SEQ ID NO: 11 - protein, 10 aa, SARS-CoV-2
VTPSGTWLTY

SEQ ID NO: 12 - protein, 19 aa, SARS-CoV-2
TYTGAIKLDDKDPNFKDQV

SEQ ID NO: 13 - protein, 9 aa, SARS-CoV-2
TPSGTWLTY

SEQ ID NO: 14 - protein, 10 aa, SARS-CoV-2
MEVTPSGTWL

SEQ ID NO: 15 - protein, 19 aa, SARS-CoV-2
GMSRIGMEVTPSGTWLTYT

SEQ ID NO: 16 - protein, 11 aa, SARS-CoV-2
GMEVTPSGTWL

SEQ ID NO: 17 - protein, 19 aa, SARS-CoV-2
DQVILLNKHIDAYKTFPPT

SEQ ID NO: 18 - protein, 19 aa, SARS-CoV-2
DDKDPNFKDQVILLNKHID

SEQ ID NO: 19 - protein, 9 aa, SARS-CoV-2
SPRWYFYYL

SEQ ID NO: 20 - protein, 9 aa, SARS-CoV-2
LSPRWYFYY

SEQ ID NO: 21 - protein, 11 aa, SARS-CoV-2
HTTDPSFLGRY

SEQ ID NO: 22 - protein, 13 aa, SARS-CoV-2
SNEKQEILGTVSW

SEQ ID NO: 23 - protein, 9 aa, SARS-CoV-2
ILGTVSWNL

SEQ ID NO: 24 - protein, 10 aa, SARS-CoV-2
YFTSDYYQLY

SEQ ID NO: 25 - protein, 10 aa, SARS-CoV-2
VLHSYFTSDY

SEQ ID NO: 26 - protein, 10 aa, SARS-CoV-2
SYFTSDYYQL

SEQ ID NO: 27 - protein, 10 aa, SARS-CoV-2
YFLQSINFVR

SEQ ID NO: 28 - protein, 10 aa, SARS-CoV-2
VYFLQSINFV

SEQ ID NO: 29 - protein, 9 aa, SARS-CoV-2
VYFLQSINF

SEQ ID NO: 30 - protein, 10 aa, SARS-CoV-2
SINFVRIIMR

SEQ ID NO: 31 - protein, 9 aa, SARS-CoV-2
QSINFVRII

SEQ ID NO: 32 - protein, 10 aa, SARS-CoV-2
LQSINFVRII

SEQ ID NO: 33 - protein, 9 aa, SARS-CoV-2
LQSINFVRI

SEQ ID NO: 34 - protein, 9 aa, SARS-CoV-2
INFVRIIMR

SEQ ID NO: 35 - protein, 10 aa, SARS-CoV-2
GLEAPFLYLY

SEQ ID NO: 36 - protein, 10 aa, SARS-CoV-2
FLYLYALVYF

SEQ ID NO: 37 - protein, 10 aa, SARS-CoV-2
FLQSINFVRI

SEQ ID NO: 38 - protein, 9 aa, SARS-CoV-2
FLQSINFVR

SEQ ID NO: 39 - protein, 9 aa, SARS-CoV-2
YLYALVYFL

SEQ ID NO: 40 - protein, 20 aa, SARS-CoV-2
STQFAFACPDGVKHVYQLRA

SEQ ID NO: 41 - protein, 20 aa, SARS-CoV-2
GNSPFHPLADNKFALTCFST

SEQ ID NO: 42 - protein, 20 aa, SARS-CoV-2
DNKFALTCFSTQFAFACPDG

SEQ ID NO: 43 - protein, 20 aa, SARS-CoV-2
DGVKHVYQLRARSVSPKLFI

SEQ ID NO: 44 - protein, 10 aa, SARS-CoV-2
TLATCELYHY

SEQ ID NO: 45 - protein, 10 aa, SARS-CoV-2
LITLATCELY

SEQ ID NO: 46 - protein, 9 aa, SARS-CoV-2
ITLATCELY

SEQ ID NO: 47 - protein, 10 aa, SARS-CoV-2
CELYHYQECV

SEQ ID NO: 48 - protein, 9 aa, SARS-CoV-2
YLCFLAFLL

SEQ ID NO: 49 - protein, 10 aa, SARS-CoV-2
SLIDFYLCFL

SEQ ID NO: 50 - protein, 10 aa, SARS-CoV-2
MIELSLIDFY

SEQ ID NO: 51 - protein, 9 aa, SARS-CoV-2
LLFLVLIML

SEQ ID NO: 52 - protein, 9 aa, SARS-CoV-2
LIDFYLCFL

SEQ ID NO: 53 - protein, 10 aa, SARS-CoV-2
IELSLIDFYL

SEQ ID NO: 54 - protein, 10 aa, SARS-CoV-2
IDFYLCFLAF

SEQ ID NO: 55 - protein, 10 aa, SARS-CoV-2
FYLCFLAFLL

SEQ ID NO: 56 - protein, 9 aa, SARS-CoV-2
FYLCFLAFL

SEQ ID NO: 57 - protein, 9 aa, SARS-CoV-2
FLAFLLFLV

SEQ ID NO: 58 - protein, 9 aa, SARS-CoV-2
AFLLFLVLI

SEQ ID NO: 59 - protein, 9 aa, SARS-CoV-2
YINVFAFPF

SEQ ID NO: 60 - protein, 10 aa, SARS-CoV-2
NVFAFPFTIY

SEQ ID NO: 61 - protein, 9 aa, SARS-CoV-2
NVFAFPFTI

SEQ ID NO: 62 - protein, 9 aa, SARS-CoV-2
MGYINVFAF

SEQ ID NO: 63 - protein, 10 aa, SARS-CoV-2
INVFAFPFTI

SEQ ID NO: 64 - protein, 10 aa, SARS-CoV-2
GYINVFAFPF

SEQ ID NO: 65 - protein, 9 aa, SARS-CoV-2
AFPFTIYSL

SEQ ID NO: 66 - protein, 10 aa, SARS-CoV-2
VLPFNDGVYF

SEQ ID NO: 67 - protein, 9 aa, SARS-CoV-2
VLPFNDGVY

SEQ ID NO: 68 - protein, 10 aa, SARS-CoV-2
LPFNDGVYFA

SEQ ID NO: 69 - protein, 9 aa, SARS-CoV-2
LPFNDGVYF

SEQ ID NO: 70 - protein, 9 aa, SARS-CoV-2
GVYFASTEK

SEQ ID NO: 71 - protein, 10 aa, SARS-CoV-2
DGVYFASTEK

SEQ ID NO: 72 - protein, 19 aa, SARS-CoV-2
QILPDPSKPSKRSFIEDLL

SEQ ID NO: 73 - protein, 19 aa, SARS-CoV-2
PSKRSFIEDLLFNKVTLAD

SEQ ID NO: 74 - protein, 19 aa, SARS-CoV-2
DFGGFNFSQILPDPSKPSK

SEQ ID NO: 75 - protein, 19 aa, SARS-CoV-2
QYGDCLGDIAARDLICAQK

SEQ ID NO: 76 - protein, 19 aa, SARS-CoV-2
LADAGFIKQYGDCLGDIAA

SEQ ID NO: 77 - protein, 19 aa, SARS-CoV-2
DLLFNKVTLADAGFIKQYG

SEQ ID NO: 78 - protein, 19 aa, SARS-CoV-2
VIAWNSNNLDSKVGGNYNY

SEQ ID NO: 79 - protein, 19 aa, SARS-CoV-2
LPDDFTGCVIAWNSNNLDS

SEQ ID NO: 80 - protein, 9 aa, SARS-CoV-2
NYNYLYRLF

SEQ ID NO: 81 - protein, 9 aa, SARS-CoV-2
NYLYRLFRK

SEQ ID NO: 82 - protein, 19 aa, SARS-CoV-2
LDSKVGGNYNYLYRLFRKS

SEQ ID NO: 83 - protein, 19 aa, SARS-CoV-2
ERDISTEIYQAGSTPCNGV

SEQ ID NO: 84 - protein, 19 aa, SARS-CoV-2
YNYLYRLFRKSNLKPFERD

SEQ ID NO: 85 - protein, 9 aa, SARS-CoV-2
YFPLQSYGF

SEQ ID NO: 86 - protein, 19 aa, SARS-CoV-2
VRFPNITNLCPFGEVFNAT

SEQ ID NO: 87 - protein, 9 aa, SARS-CoV-2
VRFPNITNL

SEQ ID NO: 88 - protein, 10 aa, SARS-CoV-2
VQPTESIVRF

SEQ ID NO: 89 - protein, 10 aa, SARS-CoV-2
TESIVRFPNI

SEQ ID NO: 90 - protein, 19 aa, SARS-CoV-2
RVQPTESIVRFPNITNLCP

SEQ ID NO: 91 - protein, 11 aa, SARS-CoV-2
RFPNITNLCPF

SEQ ID NO: 92 - protein, 9 aa, SARS-CoV-2
QPTESIVRF

SEQ ID NO: 93 - protein, 10 aa, SARS-CoV-2
FPNITNLCPF

SEQ ID NO: 94 - protein, 19 aa, SARS-CoV-2
YAWNRKRISNCVADYSVLY

SEQ ID NO: 95 - protein, 19 aa, SARS-CoV-2
SNCVADYSVLYNSASFSTF

SEQ ID NO: 96 - protein, 19 aa, SARS-CoV-2
NATRFASVYAWNRKRISNC

SEQ ID NO: 97 - protein, 19 aa, SARS-CoV-2
SELVIGAVILRGHLRIAGH

SEQ ID NO: 98 - protein, 10 aa, SARS-CoV-2
SELVIGAVIL

SEQ ID NO: 99 - protein, 9 aa, SARS-CoV-2
SELVIGAVI

SEQ ID NO: 100 - protein, 19 aa, SARS-CoV-2
LNVPLHGTILTRPLLESEL

SEQ ID NO: 101 - protein, 19 aa, SARS-CoV-2
ILTRPLLESELVIGAVILR

SEQ ID NO: 102 - protein, 19 aa, SARS-CoV-2
FNPETNILLNVPLHGTILT

SEQ ID NO: 103 - protein, 10 aa, SARS-CoV-2
WPVTLACFVL

SEQ ID NO: 104 - protein, 9 aa, SARS-CoV-2
WLLWPVTLA

SEQ ID NO: 105 - protein, 10 aa, SARS-CoV-2
TLACFVLAAV

SEQ ID NO: 106 - protein, 9 aa, SARS-CoV-2
LWPVTLACF

SEQ ID NO: 107 - protein, 9 aa, SARS-CoV-2
LWLLWPVTL

SEQ ID NO: 108 - protein, 10 aa, SARS-CoV-2
FLWLLWPVTL

SEQ ID NO: 109 - protein, 9 aa, SARS-CoV-2
FLWLLWPVT

SEQ ID NO: 110 - protein, 13 aa, SARS-CoV-2
NRDVDTDFVNEFY

SEQ ID NO: 111 - protein, 11 aa, SARS-CoV-2
DTDFVNEFYAY

SEQ ID NO: 112 - protein, 11 aa, SARS-CoV-2
KVPTDNYITTY

SEQ ID NO: 113 - protein, 14 aa, SARS-CoV-2
ALRKVPTDNYITTY

SEQ ID NO: 114 - protein, 19 aa, SARS-CoV-2
YTEKWESGVKDCVVLHSYF

SEQ ID NO: 115 - protein, 19 aa, SARS-CoV-2
EHDYQIGGYTEKWESGVKD

SEQ ID NO: 116 - protein, 19 aa, SARS-CoV-2
VKDCVVLHSYFTSDYYQLY

SEQ ID NO: 117 - protein, 19 aa, SARS-CoV-2
SYFTSDYYQLYSTQLSTDT

SEQ ID NO: 118 - protein, 19 aa, SARS-CoV-2
QLYSTQLSTDTGVEHVTFF

SEQ ID NO: 119 - protein, 9 aa, SARS-CoV-2
ALSKGVHFV

SEQ ID NO: 120 - protein, 9 aa, SARS-CoV-2
VEHVTFFIY

SEQ ID NO: 121 - protein, 14 aa, SARS-CoV-2
STDTGVEHVTFFIY

SEQ ID NO: 122 - protein, 9 aa, SARS-CoV-2
HVTFFIYNK

SEQ ID NO: 123 - protein, 10 aa, SARS-CoV-2
GVEHVTFFIY

SEQ ID NO: 124 - protein, 9 aa, SARS-CoV-2
YDANYFLCW

SEQ ID NO: 125 - protein, 10 aa, SARS-CoV-2
PLLYDANYFL

SEQ ID NO: 126 - protein, 9 aa, SARS-CoV-2
NPLLYDANY

SEQ ID NO: 127 - protein, 10 aa, SARS-CoV-2
LYDANYFLCW

SEQ ID NO: 128 - protein, 10 aa, SARS-CoV-2
LLYDANYFLC

SEQ ID NO: 129 - protein, 9 aa, SARS-CoV-2
LLYDANYFL

SEQ ID NO: 130 - protein, 10 aa, SARS-CoV-2
VQELYSPIFL

SEQ ID NO: 131 - protein, 9 aa, SARS-CoV-2
VQELYSPIF

SEQ ID NO: 132 - protein, 9 aa, SARS-CoV-2
QELYSPIFL

SEQ ID NO: 133 - protein, 9 aa, SARS-CoV-2
LYSPIFLIV

SEQ ID NO: 134 - protein, 9 aa, SARS-CoV-2
ELYSPIFLI

SEQ ID NO: 135 - protein, 20 aa, SARS-CoV-2
YSKWYIRVGARKSAPLIELC

SEQ ID NO: 136 - protein, 20 aa, SARS-CoV-2
LCVDEAGSKSPIQYIDIGNY

SEQ ID NO: 137 - protein, 20 aa, SARS-CoV-2
ARKSAPLIELCVDEAGSKSP

SEQ ID NO: 138 - protein, 11 aa, SARS-CoV-2
YYVGYLQPRTF

SEQ ID NO: 139 - protein, 9 aa, SARS-CoV-2
YLQPRTFLL

SEQ ID NO: 140 - protein, 8 aa, SARS-CoV-2
YLQPRTFL

SEQ ID NO: 141 - protein, 19 aa, SARS-CoV-2
NLLLQYGSFCTQLNRALTG

SEQ ID NO: 142 - protein, 19 aa, SARS-CoV-2
FCTQLNRALTGIAVEQDKN

SEQ ID NO: 143 - protein, 19 aa, SARS-CoV-2
CGDSTECSNLLLQYGSFCT

SEQ ID NO: 144 - protein, 19 aa, SARS-CoV-2
LTGIAVEQDKNTQEVFAQV

SEQ ID NO: 145 - protein, 19 aa, SARS-CoV-2
DKNTQEVFAQVKQIYKTPP

SEQ ID NO: 146 - protein, 19 aa, SARS-CoV-2
AQVKQIYKTPPIKDFGGFN

SEQ ID NO: 147 - protein, 19 aa, SARS-CoV-2
PLVSSQCVNLTTRTQLPPA

SEQ ID NO: 148 - protein, 19 aa, SARS-CoV-2
NLTTRTQLPPAYTNSFTRG

SEQ ID NO: 149 - protein, 19 aa, SARS-CoV-2
MFVFLVLLPLVSSQCVNLT

SEQ ID NO: 150 - protein, 19 aa, SARS-CoV-2
TRGVYYPDKVFRSSVLHST

SEQ ID NO: 151 - protein, 19 aa, SARS-CoV-2
PPAYTNSFTRGVYYPDKVF

SEQ ID NO: 152 - protein, 19 aa, SARS-CoV-2
KVFRSSVLHSTQDLFLPFF

SEQ ID NO: 153 - protein, 19 aa, SARS-CoV-2
YSSANNCTFEYVSQPFLMD

SEQ ID NO: 154 - protein, 10 aa, SARS-CoV-2
VYSSANNCTF

SEQ ID NO: 155 - protein, 10 aa, SARS-CoV-2
SSANNCTFEY

SEQ ID NO: 156 - protein, 19 aa, SARS-CoV-2
LMDLEGKQGNFKNLREFVF

SEQ ID NO: 157 - protein, 19 aa, SARS-CoV-2
FEYVSQPFLMDLEGKQGNF

SEQ ID NO: 158 - protein, 19 aa, SARS-CoV-2
YFKIYSKHTPINLVRDLPQ

SEQ ID NO: 159 - protein, 19 aa, SARS-CoV-2
GNFKNLREFVFKNIDGYFK

SEQ ID NO: 160 - protein, 19 aa, SARS-CoV-2
FVFKNIDGYFKIYSKHTPI

SEQ ID NO: 161 - protein, 1432 aa, Synthetic construct
SEHDYQIGGYTEKWESGVKDCVVLHSYFTSDYYQLYSTQLSTDTGVEHVTFFIGGGGSGGGGSLPNNTASWFTALTQHGKEDLKFPRGQGVPINTNSSPDDQIGYYRRATRRIRGGSSGGGSSGGGHLRIAGHHLGRCDIKDLPKEITVATSRTLSYYKLGASQRVAGDSGGGSSGGGSSGSTQLSTDTGVEHVTFFIYNKIVDEPEGGGGSGGGGSDGKMKDLSPRWYFYYLGTGPEAGGGGSGGGGSVEGFNCYFPLQSYGFQPTNGVGGGGSGGGGSKRFDNPVLPFNDGVYFASTEKSNIIRGSSGGGSSGGGFGMSRIGMEVTPSGTWLTYTGAIKLDDKDPNFKDQVILLNKHIDAYKTFPPTEGGGGSGGGGSAGAAAYYVGYLQPRTFLLKYNENGSSGGGSSGGGPSIISNEKQEILGTVSWNLREMLAHGGSGGGGSGGEGNSPFHPLADNKFALTCFSTQFAFACPDGVKHVYQLRARSVSPKLFIGGGGSGGGGSMFVFLVLLPLVSSQCVNLTTRTQLPPAYTNSFTRGVYYPDKVFRSSVLHSTQDLFLPFFSSGGGGSGGGGKKRWQLALSKGVHFVCNLLLLSGGGGSGGGGFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSSFNPETNILLNVPLHGTILTRPLLESELVIGAVILRGHLRIAGHHGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSMLAKALRKVPTDNYITTYPGQGLGSGGGGSGGGQTSNFRVQPTESIVRFPNITNLCPFGEVFNATRFASVYAWNRKRISNCVADYSVLYNSASFSTFKGGGGSGGGGSFYSKWYIRVGARKSAPLIELCVDEAGSKSPIQYIDIGNYSGGGGSGGGGAFEYYHTTDPSFLGRYMSALNGGGGSGGGGSMESEFRVYSSANNCTFEYVSQPFLMDLEGKQGNFKNLREFVFKNIDGYFKIYSKHTPINLVRDLPQGSSGGGSSGGGICGDSTECSNLLLQYGSFCTQLNRALTGIAVEQDKNTQEVFAQVKQIYKTPPIKDFGGFNFGGGGSGGGGSCLYRNRDVDTDFVNEFYAYLRKHFGGGGSGGGGSKLPDDFTGCVIAWNSNNLDSKVGGNYNYLYRLFRKSNLKPFERDISTEIYQAGSTPCNGVEGGGGSGGGGSWKCRSKNPLLYDANYFLCWHTNCYD

SEQ ID NO: 162 - protein, 1630 aa, Synthetic construct
SEHDYQIGGYTEKWESGVKDCVVLHSYFTSGGGGSGGGGNLDSKVGGNYNYLYRLFRKSNLKPFERDISTEIYQAGSTPCNGVEGGGGSGGGGSWKCRSKNPLLYDANYFLCWHTNCYDGGGGSGGGGSHLRIAGHHLGRCDIKDLPKEITVATSRTLSYYKLGASQRVAGDSGSSGGGSSGGGKLPDDFTGCVIAWNSNNLDSKVGGNYNYLSGGGGSGGGGDGKMKDLSPRWYFYYLGTGPEAGGSGGGGSGGQGNFKNLREFVFKNIDGYFKIYSKHTPINLVRDLPQGSSGGGSSGGGVEGFNCYFPLQSYGFQPTNGVGGGGSGGGGSFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLDDKDPNFKDQVILLNKHIDAYKTFPPTEGGGGSGGGGSQTSNFRVQPTESIVRFPNITNLCPFGEVFNATRSGGGGSGGGGMLAKALRKVPTDNYITTYPGQGLGSGGGGSGGGPPAYTNSFTRGVYYPDKVFRSSVLHSTQDLFLPFFSSGGGGSGGGGKDFGGFNFSQILPDPSKPSKRSFIEDLLFNKVTLADAGGGGSGGGGSIILFLALITLATCELYHYQECVRGTTVGGGGSGGGGSKKRWQLALSKGVHFVCNLLLLSGGGGSGGGGMGYINVFAFPFTIYSLLLCRMNGGGGSGGGGSLLLVAAGLEAPFLYLYALVYFLQSINFVRIIMRLWLCWKGGGGSGGGGSMIELSLIDFYLCFLAFLLFLVLIMLIIFWFSSGGGGSGGGGYIIKLIFLWLLWPVTLACFVLAAVYRINWGGGGSGGGGSFIRQEEVQELYSPIFLIVAAIVFIGGGGSGGGGSMFVFLVLLPLVSSQCVNLTTRTQLPPAYTNSFTRGVGGGGSGGGGSSFNPETNILLNVPLHGTILTRPLLESELVIGAVILRGHLRIAGHHGGGGSGGGGSEGNSPFHPLADNKFALTCFSTQFAFACPDGVKHVYQLRARSVSPKLFIGGGGSGGGGSEDLLFNKVTLADAGFIKQYGDCLGDIAARDLICAQKFGGGGSGGGGSPSIISNEKQEILGTVSWNLREMLAHSGGGGSGGGGALTGIAVEQDKNTQEVFAQVKQIYKTPPIKDFGGFNFGGGGSGGGGSAGAAAYYVGYLQPRTFLLKYNENGSSGGGSSGGGLPNNTASWFTALTQHGKEDLKFPRGQGVPGGGGSGGGGSKRFDNPVLPFNDGVYFASTEKSNIIRGSSGGGSSGGGFNATRFASVYAWNRKRISNCVADYSVLYNSASFSTFKGGGGSGGGGSAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSCLYRNRDVDTDFVNEFYAYLRKHFGGGGSGGGGSGVKDCVVLHSYFTSDYYQLYSTQLSTDTGVEHVTFFIGGGGSGGGGSICGDSTECSNLLLQYGSFCTQLNRALTGIAVEQDKNTSGGGGSGGGGKEDLKFPRGQGVPINTNSSPDDQIGYYRRATRRIRGG

SEQ ID NO: 163 - protein, 1415 aa, Synthetic construct
SEHDYQIGGYTEKWESGVKDCVVLHSYFTSGGGGSGGGGICGDSTECSNLLLQYGSFCTQLNRALTGIAVEQDKNTGSGGGGSGGGGVKDCVVLHSYFTSDYYQLYSTQLSTDTGVEHVTFFIGGGGSGGGGSCLYRNRDVDTDFVNEFYAYLRKHFGGGGSGGGGSDGKMKDLSPRWYFYYLGTGPEAGGSGGGGSGGQGNFKNLREFVFKNIDGYFKIYSKHTPINLVRDLPQGSSGGGSSGGGFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLPNNTASWFTALTQHGKEDLKFPRGQGVPGGGGSGGGGSALTGIAVEQDKNTQEVFAQVKQIYKTPPIKDFGGFNFGGGGSGGGGSPSIISNEKQEILGTVSWNLREMLAHSGGGGSGGGGKDFGGFNFSQILPDPSKPSKRSFIEDLLFNKVTLADAGGGGSGGGGSEGNSPFHPLADNKFALTCFSTQFAFACPDGVKHVYQLRARSVSPKLFIGGGGSGGGGSSFNPETNILLNVPLHGTILTRPLLESELVIGAVILRGHLRIAGHHGGGGSGGGGSMFVFLVLLPLVSSQCVNLTTRTQLPPAYTNSFTRGVGGGGSGGGGSFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSKKRWQLALSKGVHFVCNLLLLSGGGGSGGGGIILFLALITLATCELYHYQECVRGTTVGGGGSGGGGSEDLLFNKVTLADAGFIKQYGDCLGDIAARDLICAQKFGGGGSGGGGSPPAYTNSFTRGVYYPDKVFRSSVLHSTQDLFLPFFSSGGGGSGGGGMLAKALRKVPTDNYITTYPGQGLGGGSGGGGSGAGAAAYYVGYLQPRTFLLKYNENGSSGGGSSGGGLDDKDPNFKDQVILLNKHIDAYKTFPPTEGGGGSGGGGSKRFDNPVLPFNDGVYFASTEKSNIIRGSSGGGSSGGGAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSHLRIAGHHLGRCDIKDLPKEITVATSRTLSYYKLGASQRVAGDSGSSGGGSSGGGWKCRSKNPLLYDANYFLCWHTNCYDGGGGSGGGGSKEDLKFPRGQGVPINTNSSPDDQIGYYRRATRRIRGG

SEQ ID NO: 164 - protein, 806 aa, Synthetic construct
GVKDCVVLHSYFTSDYYQLYSTQLSSGGGGSGGGGKLPDDFTGCVIAWNSNNLDSKVGGNYNYLYRLFRKSNLKPFERDISTEIYQAGSTPCNGVEGGGGSGGGGSDGKMKDLSPRWYFYYLGTGPEAGGGGSGGGGSVEGFNCYFPLQSYGFQPTNGVGGGGSGGGGSFGMSRIGMEVTPSGTWLTYTGAIKLDDKDPNFKDQVILLNKHIDAYKTFPPTEGGGGSGGGGSPSIISNEKQEILGTVSWNLREMLAHGGSGGGGSGGEGNSPFHPLADNKFALTCFSTQFAFACPDGVKHVYQLRARSVSPKLFIGGGGSGGGGSMGYINVFAFPFTIYSLLLCRMNGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSQTSNFRVQPTESIVRFPNITNLCPFGEVFNATRFASVYAWNRKRISNCVADYSVLYNSASFSTFKGGGGSGGGGSKRFDNPVLPFNDGVYFASTEKSNIIRGSSGGGSSGGGAFEYYHTTDPSFLGRYMSALNGGGGSGGGGSHLRIAGHHLGRCDIKDLPKEITVATSRTLSYYKLGASQRVAGDSGSSGGGSSGGGLPNNTASWFTALTQHGKEDLKFPRGQGVPINTNSSPDDQIGYYRRATRRIRGG

SEQ ID NO: 165 - protein, 1145 aa, Synthetic construct
MSFNPETNILLNVPLHGTILTRPLLESELVIGAVILRGHLRIAGHHSGSSGSKDFGGFNFSQILPDPSKPSKRSFIEDLLFNKVTLADAGFIKQYGDCLGDIAARDLICAQKFSGSSGSSTQLSTDTGVEHVTFFIYNKIVDEPESGSSGSMESEFRVYSSANNCTFEYVSQPFLMDLEGKQGNFKNLREFVFKNIDGYFKIYSKHTPINLVRDLPQGSGSSGSAFEYYHTTDPSFLGRYMSALNSGSSGSLLLVAAGLEAPFLYLYALVYFLQSINFVRIIMRLWLCWKSGSSGSFIRQEEVQELYSPIFLIVAAIVFISGSSGSSEHDYQIGGYTEKWESGVKDCVVLHSYFTSDYYQLYSTQLSTDTGVEHVTFFIGGSGGYIIKLIFLWLLWPVTLACFVLAAVYRINWGGSGGCLYRNRDVDTDFVNEFYAYLRKHFGGSGGKRFDNPVLPFNDGVYFASTEKSNIIRGSGSSGSFYSKWYIRVGARKSAPLIELCVDEAGSKSPIQYIDIGNYGGSGGMIELSLIDFYLCFLAFLLFLVLIMLIIFWFSGGSGGICGDSTECSNLLLQYGSFCTQLNRALTGIAVEQDKNTQEVFAQVKQIYKTPPIKDFGGFNFSGSSGSHLRIAGHHLGRCDIKDLPKEITVATSRTLSYYKLGASQRVAGDSGGGSGGDGKMKDLSPRWYFYYLGTGPEAGGSGGPSIISNEKQEILGTVSWNLREMLAHGGSGGWKCRSKNPLLYDANYFLCWHTNCYDGGSGGKKRWQLALSKGVHFVCNLLLLSGSSGSMLAKALRKVPTDNYITTYPGQGLSGSSGSMGYINVFAFPFTIYSLLLCRMNGGSGGMFVFLVLLPLVSSQCVNLTTRTQLPPAYTNSFTRGVYYPDKVFRSSVLHSTQDLFLPFFSGGSGGAGAAAYYVGYLQPRTFLLKYNENGGGSGGEGNSPFHPLADNKFALTCFSTQFAFACPDGVKHVYQLRARSVSPKLFIGGSGGFGMSRIGMEVTPSGTWLTYTGAIKLDDKDPNFKDQVILLNKHIDAYKTFPPTESGSSGSIILFLALITLATCELYHYQECVRGTTVSGSSGSLPNNTASWFTALTQHGKEDLKFPRGQGVPINTNSSPDDQIGYYRRATRRIRGGSGSSGS

SEQ ID NO: 166 - protein, 1273 aa, Synthetic construct
MCLYRNRDVDTDFVNEFYAYLRKHFSGSSGSKDFGGFNFSQILPDPSKPSKRSFIEDLLFNKVTLADASGSSGSAFEYYHTTDPSFLGRYMSALNGGSGGEDLLFNKVTLADAGFIKQYGDCLGDIAARDLICAQKFGGSGGICGDSTECSNLLLQYGSFCTQLNRALTGIAVEQDKNTGGSGGDGKMKDLSPRWYFYYLGTGPEAGGSGGKRFDNPVLPFNDGVYFASTEKSNIIRGSGSSGSMESEFRVYSSANNCTFEYVSQPFLMDLEGKQGNFKNLREFVFKSGSSGSMIELSLIDFYLCFLAFLLFLVLIMLIIFWFSSGSSGSFIRQEEVQELYSPIFLIVAAIVFISGSSGSKKRWQLALSKGVHFVCNLLLLSGSSGSIILFLALITLATCELYHYQECVRGTTVGGSGGMFVFLVLLPLVSSQCVNLTTRTQLPPAYTNSFTRGVGGSGGSFNPETNILLNVPLHGTILTRPLLESELVIGAVILRGHLRIAGHHGGSGGMLAKALRKVPTDNYITTYPGQGLSGSSGSLLLVAAGLEAPFLYLYALVYFLQSINFVRIIMRLWLCWKGGSGGLDDKDPNFKDQVILLNKHIDAYKTFPPTEGGSGGYIIKLIFLWLLWPVTLACFVLAAVYRINWSGSSGSQGNFKNLREFVFKNIDGYFKIYSKHTPINLVRDLPQGSGSSGSPPAYTNSFTRGVYYPDKVFRSSVLHSTQDLFLPFFSSGSSGSGVKDCVVLHSYFTSDYYQLYSTQLSTDTGVEHVTFFISGSSGSALTGIAVEQDKNTQEVFAQVKQIYKTPPIKDFGGFNFGGSGGAGAAAYYVGYLQPRTFLLKYNENGSGSSGSFYSKWYIRVGARKSAPLIELCVDEAGSKSPIQYIDIGNYGGSGGWKCRSKNPLLYDANYFLCWHTNCYDGGSGGMGYINVFAFPFTIYSLLLCRMNSGSSGSLPNNTASWFTALTQHGKEDLKFPRGQGVPGGSGGFGMSRIGMEVTPSGTWLTYTGAIKLDDKDPNFKDQVISGSSGSEGNSPFHPLADNKFALTCFSTQFAFACPDGVKHVYQLRARSVSPKLFIGGSGGSEHDYQIGGYTEKWESGVKDCVVLHSYFTSGSSGSKEDLKFPRGQGVPINTNSSPDDQIGYYRRATRRIRGGGGSGGSTQLSTDTGVEHVTFFIYNKIVDEPEGGSGGHLRIAGHHLGRCDIKDLPKEITVATSRTLSYYKLGASQRVAGDSGGGSGGPSIISNEKQEILGTVSWNLREMLAHSGSSGS

SEQ ID NO: 167 - protein, 825 aa, Synthetic construct
MFGMSRIGMEVTPSGTWLTYTGAIKLDDKDPNFKDQVIGGSGGQTSNFRVQPTESIVRFPNITNLCPFGEVFNATRSGSSGSMGYINVFAFPFTIYSLLLCRMNGGSGGFNATRFASVYAWNRKRISNCVADYSVLYNSASFSTFKSGSSGSNLDSKVGGNYNYLYRLFRKSNLKPFERDISTEIYQAGSTPCNGVESGSSGSVEGFNCYFPLQSYGFQPTNGVGGSGGLDDKDPNFKDQVILLNKHIDAYKTFPPTESGSSGSGVKDCVVLHSYFTSDYYQLYSTQLSSGSSGSEGNSPFHPLADNKFALTCFSTQFAFACPDGVKHVYQLRARSVSPKLFISGSSGSDGKMKDLSPRWYFYYLGTGPEASGSSGSMIELSLIDFYLCFLAFLLFLVLIMLIIFWFSGGSGGAFEYYHTTDPSFLGRYMSALNSGSSGSKLPDDFTGCVIAWNSNNLDSKVGGNYNYLGGSGGKRFDNPVLPFNDGVYFASTEKSNIIRGGGSGGLLLVAAGLEAPFLYLYALVYFLQSINFVRIIMRLWLCWKGGSGGEDLLFNKVTLADAGFIKQYGDCLGDIAARDLICAQKFGGSGGIILFLALITLATCELYHYQECVRGTTVSGSSGSHLRIAGHHLGRCDIKDLPKEITVATSRTLSYYKLGASQRVAGDSGGGSGGLPNNTASWFTALTQHGKEDLKFPRGQGVPGGSGGKDFGGFNFSQILPDPSKPSKRSFIEDLLFNKVTLADASGSSGSKEDLKFPRGQGVPINTNSSPDDQIGYYRRATRRIRGGSGSSGSPSIISNEKQEILGTVSWNLREMLAHGGSGG

SEQ ID NO: 168 - protein, 242 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGL

SEQ ID NO: 169 - protein, 260 aa, Synthetic construct
MNFGLRLIFLVLTLKGVQCDIQMTQTTSSLSASLGDRVTISCSASQDINNYLNWYQQKPDGTVKLLIYYTSSLHSGVPSRFSGSGSGTDYSLTISNLEPEDIATYYCQQYSKFPRTFGGGTKLEIKRGGGGSGGGGSGGGGSQIQLVQSGPELKKPGETVKISCKASGYTFINYGMNWVKQTPGKGLKWMGWINTYSGEPTYPDDFKGRFAFSLETSASTAYLQLNNLKNEDMATYFCARGDYYGPFDNWGQGTTLTVSS

SEQ ID NO: 170 - protein, 22 aa, Homo sapiens
MDAMKRGLCCVLLLCGAVFVSP

SEQ ID NO: 171 - protein, 1651 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSDYYQLYSTQLSTDTGVEHVTFFIGGGGSGGGGSLPNNTASWFTALTQHGKEDLKFPRGQGVPINTNSSPDDQIGYYRRATRRIRGGSSGGGSSGGGHLRIAGHHLGRCDIKDLPKEITVATSRTLSYYKLGASQRVAGDSGGGSSGGGSSGSTQLSTDTGVEHVTFFIYNKIVDEPEGGGGSGGGGSDGKMKDLSPRWYFYYLGTGPEAGGGGSGGGGSVEGFNCYFPLQSYGFQPTNGVGGGGSGGGGSKRFDNPVLPFNDGVYFASTEKSNIIRGSSGGGSSGGGFGMSRIGMEVTPSGTWLTYTGAIKLDDKDPNFKDQVILLNKHIDAYKTFPPTEGGGGSGGGGSAGAAAYYVGYLQPRTFLLKYNENGSSGGGSSGGGPSIISNEKQEILGTVSWNLREMLAHGGSGGGGSGGEGNSPFHPLADNKFALTCFSTQFAFACPDGVKHVYQLRARSVSPKLFIGGGGSGGGGSMFVFLVLLPLVSSQCVNLTTRTQLPPAYTNSFTRGVYYPDKVFRSSVLHSTQDLFLPFFSSGGGGSGGGGKKRWQLALSKGVHFVCNLLLLSGGGGSGGGGFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSSFNPETNILLNVPLHGTILTRPLLESELVIGAVILRGHLRIAGHHGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSMLAKALRKVPTDNYITTYPGQGLGSGGGGSGGGQTSNFRVQPTESIVRFPNITNLCPFGEVFNATRFASVYAWNRKRISNCVADYSVLYNSASFSTFKGGGGSGGGGSFYSKWYIRVGARKSAPLIELCVDEAGSKSPIQYIDIGNYSGGGGSGGGGAFEYYHTTDPSFLGRYMSALNGGGGSGGGGSMESEFRVYSSANNCTFEYVSQPFLMDLEGKQGNFKNLREFVFKNIDGYFKIYSKHTPINLVRDLPQGSSGGGSSGGGICGDSTECSNLLLQYGSFCTQLNRALTGIAVEQDKNTQEVFAQVKQIYKTPPIKDFGGFNFGGGGSGGGGSCLYRNRDVDTDFVNEFYAYLRKHFGGGGSGGGGSKLPDDFTGCVIAWNSNNLDSKVGGNYNYLYRLFRKSNLKPFERDISTEIYQAGSTPCNGVEGGGGSGGGGSWKCRSKNPLLYDANYFLCWHTNCYD

SEQ ID NO: 172 - protein, 1849 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSGGGGSGGGGNLDSKVGGNYNYLYRLFRKSNLKPFERDISTEIYQAGSTPCNGVEGGGGSGGGGSWKCRSKNPLLYDANYFLCWHTNCYDGGGGSGGGGSHLRIAGHHLGRCDIKDLPKEITVATSRTLSYYKLGASQRVAGDSGSSGGGSSGGGKLPDDFTGCVIAWNSNNLDSKVGGNYNYLSGGGGSGGGGDGKMKDLSPRWYFYYLGTGPEAGGSGGGGSGGQGNFKNLREFVFKNIDGYFKIYSKHTPINLVRDLPQGSSGGGSSGGGVEGFNCYFPLQSYGFQPTNGVGGGGSGGGGSFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLDDKDPNFKDQVILLNKHIDAYKTFPPTEGGGGSGGGGSQTSNFRVQPTESIVRFPNITNLCPFGEVFNATRSGGGGSGGGGMLAKALRKVPTDNYITTYPGQGLGSGGGGSGGGPPAYTNSFTRGVYYPDKVFRSSVLHSTQDLFLPFFSSGGGGSGGGGKDFGGFNFSQILPDPSKPSKRSFIEDLLFNKVTLADAGGGGSGGGGSIILFLALITLATCELYHYQECVRGTTVGGGGSGGGGSKKRWQLALSKGVHFVCNLLLLSGGGGSGGGGMGYINVFAFPFTIYSLLLCRMNGGGGSGGGGSLLLVAAGLEAPFLYLYALVYFLQSINFVRIIMRLWLCWKGGGGSGGGGSMIELSLIDFYLCFLAFLLFLVLIMLIIFWFSSGGGGSGGGGYIIKLIFLWLLWPVTLACFVLAAVYRINWGGGGSGGGGSFIRQEEVQELYSPIFLIVAAIVFIGGGGSGGGGSMFVFLVLLPLVSSQCVNLTTRTQLPPAYTNSFTRGVGGGGSGGGGSSFNPETNILLNVPLHGTILTRPLLESELVIGAVILRGHLRIAGHHGGGGSGGGGSEGNSPFHPLADNKFALTCFSTQFAFACPDGVKHVYQLRARSVSPKLFIGGGGSGGGGSEDLLFNKVTLADAGFIKQYGDCLGDIAARDLICAQKFGGGGSGGGGSPSIISNEKQEILGTVSWNLREMLAHSGGGGSGGGGALTGIAVEQDKNTQEVFAQVKQIYKTPPIKDFGGFNFGGGGSGGGGSAGAAAYYVGYLQPRTFLLKYNENGSSGGGSSGGGLPNNTASWFTALTQHGKEDLKFPRGQGVPGGGGSGGGGSKRFDNPVLPFNDGVYFASTEKSNIIRGSSGGGSSGGGFNATRFASVYAWNRKRISNCVADYSVLYNSASFSTFKGGGGSGGGGSAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSCLYRNRDVDTDFVNEFYAYLRKHFGGGGSGGGGSGVKDCVVLHSYFTSDYYQLYSTQLSTDTGVEHVTFFIGGGGSGGGGSICGDSTECSNLLLQYGSFCTQLNRALTGIAVEQDKNTSGGGGSGGGGKEDLKFPRGQGVPINTNSSPDDQIGYYRRATRRIRGG

SEQ ID NO: 173 - protein, 1634 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSGGGGSGGGGICGDSTECSNLLLQYGSFCTQLNRALTGIAVEQDKNTGSGGGGSGGGGVKDCVVLHSYFTSDYYQLYSTQLSTDTGVEHVTFFIGGGGSGGGGSCLYRNRDVDTDFVNEFYAYLRKHFGGGGSGGGGSDGKMKDLSPRWYFYYLGTGPEAGGSGGGGSGGQGNFKNLREFVFKNIDGYFKIYSKHTPINLVRDLPQGSSGGGSSGGGFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLPNNTASWFTALTQHGKEDLKFPRGQGVPGGGGSGGGGSALTGIAVEQDKNTQEVFAQVKQIYKTPPIKDFGGFNFGGGGSGGGGSPSIISNEKQEILGTVSWNLREMLAHSGGGGSGGGGKDFGGFNFSQILPDPSKPSKRSFIEDLLFNKVTLADAGGGGSGGGGSEGNSPFHPLADNKFALTCFSTQFAFACPDGVKHVYQLRARSVSPKLFIGGGGSGGGGSSFNPETNILLNVPLHGTILTRPLLESELVIGAVILRGHLRIAGHHGGGGSGGGGSMFVFLVLLPLVSSQCVNLTTRTQLPPAYTNSFTRGVGGGGSGGGGSFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSKKRWQLALSKGVHFVCNLLLLSGGGGSGGGGIILFLALITLATCELYHYQECVRGTTVGGGGSGGGGSEDLLFNKVTLADAGFIKQYGDCLGDIAARDLICAQKFGGGGSGGGGSPPAYTNSFTRGVYYPDKVFRSSVLHSTQDLFLPFFSSGGGGSGGGGMLAKALRKVPTDNYITTYPGQGLGGGSGGGGSGAGAAAYYVGYLQPRTFLLKYNENGSSGGGSSGGGLDDKDPNFKDQVILLNKHIDAYKTFPPTEGGGGSGGGGSKRFDNPVLPFNDGVYFASTEKSNIIRGSSGGGSSGGGAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSHLRIAGHHLGRCDIKDLPKEITVATSRTLSYYKLGASQRVAGDSGSSGGGSSGGGWKCRSKNPLLYDANYFLCWHTNCYDGGGGSGGGGSKEDLKFPRGQGVPINTNSSPDDQIGYYRRATRRIRGG

SEQ ID NO: 174 - protein, 1025 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLGVKDCVVLHSYFTSDYYQLYSTQLSSGGGGSGGGGKLPDDFTGCVIAWNSNNLDSKVGGNYNYLYRLFRKSNLKPFERDISTEIYQAGSTPCNGVEGGGGSGGGGSDGKMKDLSPRWYFYYLGTGPEAGGGGSGGGGSVEGFNCYFPLQSYGFQPTNGVGGGGSGGGGSFGMSRIGMEVTPSGTWLTYTGAIKLDDKDPNFKDQVILLNKHIDAYKTFPPTEGGGGSGGGGSPSIISNEKQEILGTVSWNLREMLAHGGSGGGGSGGEGNSPFHPLADNKFALTCFSTQFAFACPDGVKHVYQLRARSVSPKLFIGGGGSGGGGSMGYINVFAFPFTIYSLLLCRMNGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSQTSNFRVQPTESIVRFPNITNLCPFGEVFNATRFASVYAWNRKRISNCVADYSVLYNSASFSTFKGGGGSGGGGSKRFDNPVLPFNDGVYFASTEKSNIIRGSSGGGSSGGGAFEYYHTTDPSFLGRYMSALNGGGGSGGGGSHLRIAGHHLGRCDIKDLPKEITVATSRTLSYYKLGASQRVAGDSGSSGGGSSGGGLPNNTASWFTALTQHGKEDLKFPRGQGVPINTNSSPDDQIGYYRRATRRIRGG

SEQ ID NO: 175 - protein, 1364 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMSFNPETNILLNVPLHGTILTRPLLESELVIGAVILRGHLRIAGHHSGSSGSKDFGGFNFSQILPDPSKPSKRSFIEDLLFNKVTLADAGFIKQYGDCLGDIAARDLICAQKFSGSSGSSTQLSTDTGVEHVTFFIYNKIVDEPESGSSGSMESEFRVYSSANNCTFEYVSQPFLMDLEGKQGNFKNLREFVFKNIDGYFKIYSKHTPINLVRDLPQGSGSSGSAFEYYHTTDPSFLGRYMSALNSGSSGSLLLVAAGLEAPFLYLYALVYFLQSINFVRIIMRLWLCWKSGSSGSFIRQEEVQELYSPIFLIVAAIVFISGSSGSSEHDYQIGGYTEKWESGVKDCVVLHSYFTSDYYQLYSTQLSTDTGVEHVTFFIGGSGGYIIKLIFLWLLWPVTLACFVLAAVYRINWGGSGGCLYRNRDVDTDFVNEFYAYLRKHFGGSGGKRFDNPVLPFNDGVYFASTEKSNIIRGSGSSGSFYSKWYIRVGARKSAPLIELCVDEAGSKSPIQYIDIGNYGGSGGMIELSLIDFYLCFLAFLLFLVLIMLIIFWFSGGSGGICGDSTECSNLLLQYGSFCTQLNRALTGIAVEQDKNTQEVFAQVKQIYKTPPIKDFGGFNFSGSSGSHLRIAGHHLGRCDIKDLPKEITVATSRTLSYYKLGASQRVAGDSGGGSGGDGKMKDLSPRWYFYYLGTGPEAGGSGGPSIISNEKQEILGTVSWNLREMLAHGGSGGWKCRSKNPLLYDANYFLCWHTNCYDGGSGGKKRWQLALSKGVHFVCNLLLLSGSSGSMLAKALRKVPTDNYITTYPGQGLSGSSGSMGYINVFAFPFTIYSLLLCRMNGGSGGMFVFLVLLPLVSSQCVNLTTRTQLPPAYTNSFTRGVYYPDKVFRSSVLHSTQDLFLPFFSGGSGGAGAAAYYVGYLQPRTFLLKYNENGGGSGGEGNSPFHPLADNKFALTCFSTQFAFACPDGVKHVYQLRARSVSPKLFIGGSGGFGMSRIGMEVTPSGTWLTYTGAIKLDDKDPNFKDQVILLNKHIDAYKTFPPTESGSSGSIILFLALITLATCELYHYQECVRGTTVSGSSGSLPNNTASWFTALTQHGKEDLKFPRGQGVPINTNSSPDDQIGYYRRATRRIRGGSGSSGS

SEQ ID NO: 176 - protein, 1492 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMCLYRNRDVDTDFVNEFYAYLRKHFSGSSGSKDFGGFNFSQILPDPSKPSKRSFIEDLLFNKVTLADASGSSGSAFEYYHTTDPSFLGRYMSALNGGSGGEDLLFNKVTLADAGFIKQYGDCLGDIAARDLICAQKFGGSGGICGDSTECSNLLLQYGSFCTQLNRALTGIAVEQDKNTGGSGGDGKMKDLSPRWYFYYLGTGPEAGGSGGKRFDNPVLPFNDGVYFASTEKSNIIRGSGSSGSMESEFRVYSSANNCTFEYVSQPFLMDLEGKQGNFKNLREFVFKSGSSGSMIELSLIDFYLCFLAFLLFLVLIMLIIFWFSSGSSGSFIRQEEVQELYSPIFLIVAAIVFISGSSGSKKRWQLALSKGVHFVCNLLLLSGSSGSIILFLALITLATCELYHYQECVRGTTVGGSGGMFVFLVLLPLVSSQCVNLTTRTQLPPAYTNSFTRGVGGSGGSFNPETNILLNVPLHGTILTRPLLESELVIGAVILRGHLRIAGHHGGSGGMLAKALRKVPTDNYITTYPGQGLSGSSGSLLLVAAGLEAPFLYLYALVYFLQSINFVRIIMRLWLCWKGGSGGLDDKDPNFKDQVILLNKHIDAYKTFPPTEGGSGGYIIKLIFLWLLWPVTLACFVLAAVYRINWSGSSGSQGNFKNLREFVFKNIDGYFKIYSKHTPINLVRDLPQGSGSSGSPPAYTNSFTRGVYYPDKVFRSSVLHSTQDLFLPFFSSGSSGSGVKDCVVLHSYFTSDYYQLYSTQLSTDTGVEHVTFFISGSSGSALTGIAVEQDKNTQEVFAQVKQIYKTPPIKDFGGFNFGGSGGAGAAAYYVGYLQPRTFLLKYNENGSGSSGSFYSKWYIRVGARKSAPLIELCVDEAGSKSPIQYIDIGNYGGSGGWKCRSKNPLLYDANYFLCWHTNCYDGGSGGMGYINVFAFPFTIYSLLLCRMNSGSSGSLPNNTASWFTALTQHGKEDLKFPRGQGVPGGSGGFGMSRIGMEVTPSGTWLTYTGAIKLDDKDPNFKDQVISGSSGSEGNSPFHPLADNKFALTCFSTQFAFACPDGVKHVYQLRARSVSPKLFIGGSGGSEHDYQIGGYTEKWESGVKDCVVLHSYFTSGSSGSKEDLKFPRGQGVPINTNSSPDDQIGYYRRATRRIRGGGGSGGSTQLSTDTGVEHVTFFIYNKIVDEPEGGSGGHLRIAGHHLGRCDIKDLPKEITVATSRTLSYYKLGASQRVAGDSGGGSGGPSIISNEKQEILGTVSWNLREMLAHSGSSGS

SEQ ID NO: 177 - protein, 1044 aa, Synthetic construct
APLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMFGMSRIGMEVTPSGTWLTYTGAIKLDDKDPNFKDQVIGGSGGQTSNFRVQPTESIVRFPNITNLCPFGEVFNATRSGSSGSMGYINVFAFPFTIYSLLLCRMNGGSGGFNATRFASVYAWNRKRISNCVADYSVLYNSASFSTFKSGSSGSNLDSKVGGNYNYLYRLFRKSNLKPFERDISTEIYQAGSTPCNGVESGSSGSVEGFNCYFPLQSYGFQPTNGVGGSGGLDDKDPNFKDQVILLNKHIDAYKTFPPTESGSSGSGVKDCVVLHSYFTSDYYQLYSTQLSSGSSGSEGNSPFHPLADNKFALTCFSTQFAFACPDGVKHVYQLRARSVSPKLFISGSSGSDGKMKDLSPRWYFYYLGTGPEASGSSGSMIELSLIDFYLCFLAFLLFLVLIMLIIFWFSGGSGGAFEYYHTTDPSFLGRYMSALNSGSSGSKLPDDFTGCVIAWNSNNLDSKVGGNYNYLGGSGGKRFDNPVLPFNDGVYFASTEKSNIIRGGGSGGLLLVAAGLEAPFLYLYALVYFLQSINFVRIIMRLWLCWKGGSGGEDLLFNKVTLADAGFIKQYGDCLGDIAARDLICAQKFGGSGGIILFLALITLATCELYHYQECVRGTTVSGSSGSHLRIAGHHLGRCDIKDLPKEITVATSRTLSYYKLGASQRVAGDSGGGSGGLPNNTASWFTALTQHGKEDLKFPRGQGVPGGSGGKDFGGFNFSQILPDPSKPSKRSFIEDLLFNKVTLADASGSSGSKEDLKFPRGQGVPINTNSSPDDQIGYYRRATRRIRGGSGSSGSPSIISNEKQEILGTVSWNLREMLAHGGSGG

SEQ ID NO: 178 - protein, 1674 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSDYYQLYSTQLSTDTGVEHVTFFIGGGGSGGGGSLPNNTASWFTALTQHGKEDLKFPRGQGVPINTNSSPDDQIGYYRRATRRIRGGSSGGGSSGGGHLRIAGHHLGRCDIKDLPKEITVATSRTLSYYKLGASQRVAGDSGGGSSGGGSSGSTQLSTDTGVEHVTFFIYNKIVDEPEGGGGSGGGGSDGKMKDLSPRWYFYYLGTGPEAGGGGSGGGGSVEGFNCYFPLQSYGFQPTNGVGGGGSGGGGSKRFDNPVLPFNDGVYFASTEKSNIIRGSSGGGSSGGGFGMSRIGMEVTPSGTWLTYTGAIKLDDKDPNFKDQVILLNKHIDAYKTFPPTEGGGGSGGGGSAGAAAYYVGYLQPRTFLLKYNENGSSGGGSSGGGPSIISNEKQEILGTVSWNLREMLAHGGSGGGGSGGEGNSPFHPLADNKFALTCFSTQFAFACPDGVKHVYQLRARSVSPKLFIGGGGSGGGGSMFVFLVLLPLVSSQCVNLTTRTQLPPAYTNSFTRGVYYPDKVFRSSVLHSTQDLFLPFFSSGGGGSGGGGKKRWQLALSKGVHFVCNLLLLSGGGGSGGGGFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSSFNPETNILLNVPLHGTILTRPLLESELVIGAVILRGHLRIAGHHGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSMLAKALRKVPTDNYITTYPGQGLGSGGGGSGGGQTSNFRVQPTESIVRFPNITNLCPFGEVFNATRFASVYAWNRKRISNCVADYSVLYNSASFSTFKGGGGSGGGGSFYSKWYIRVGARKSAPLIELCVDEAGSKSPIQYIDIGNYSGGGGSGGGGAFEYYHTTDPSFLGRYMSALNGGGGSGGGGSMESEFRVYSSANNCTFEYVSQPFLMDLEGKQGNFKNLREFVFKNIDGYFKIYSKHTPINLVRDLPQGSSGGGSSGGGICGDSTECSNLLLQYGSFCTQLNRALTGIAVEQDKNTQEVFAQVKQIYKTPPIKDFGGFNFGGGGSGGGGSCLYRNRDVDTDFVNEFYAYLRKHFGGGGSGGGGSKLPDDFTGCVIAWNSNNLDSKVGGNYNYLYRLFRKSNLKPFERDISTEIYQAGSTPCNGVEGGGGSGGGGSWKCRSKNPLLYDANYFLCWHTNCYD

SEQ ID NO: 179 - protein, 1872 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSGGGGSGGGGNLDSKVGGNYNYLYRLFRKSNLKPFERDISTEIYQAGSTPCNGVEGGGGSGGGGSWKCRSKNPLLYDANYFLCWHTNCYDGGGGSGGGGSHLRIAGHHLGRCDIKDLPKEITVATSRTLSYYKLGASQRVAGDSGSSGGGSSGGGKLPDDFTGCVIAWNSNNLDSKVGGNYNYLSGGGGSGGGGDGKMKDLSPRWYFYYLGTGPEAGGSGGGGSGGQGNFKNLREFVFKNIDGYFKIYSKHTPINLVRDLPQGSSGGGSSGGGVEGFNCYFPLQSYGFQPTNGVGGGGSGGGGSFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLDDKDPNFKDQVILLNKHIDAYKTFPPTEGGGGSGGGGSQTSNFRVQPTESIVRFPNITNLCPFGEVFNATRSGGGGSGGGGMLAKALRKVPTDNYITTYPGQGLGSGGGGSGGGPPAYTNSFTRGVYYPDKVFRSSVLHSTQDLFLPFFSSGGGGSGGGGKDFGGFNFSQILPDPSKPSKRSFIEDLLFNKVTLADAGGGGSGGGGSIILFLALITLATCELYHYQECVRGTTVGGGGSGGGGSKKRWQLALSKGVHFVCNLLLLSGGGGSGGGGMGYINVFAFPFTIYSLLLCRMNGGGGSGGGGSLLLVAAGLEAPFLYLYALVYFLQSINFVRIIMRLWLCWKGGGGSGGGGSMIELSLIDFYLCFLAFLLFLVLIMLIIFWFSSGGGGSGGGGYIIKLIFLWLLWPVTLACFVLAAVYRINWGGGGSGGGGSFIRQEEVQELYSPIFLIVAAIVFIGGGGSGGGGSMFVFLVLLPLVSSQCVNLTTRTQLPPAYTNSFTRGVGGGGSGGGGSSFNPETNILLNVPLHGTILTRPLLESELVIGAVILRGHLRIAGHHGGGGSGGGGSEGNSPFHPLADNKFALTCFSTQFAFACPDGVKHVYQLRARSVSPKLFIGGGGSGGGGSEDLLFNKVTLADAGFIKQYGDCLGDIAARDLICAQKFGGGGSGGGGSPSIISNEKQEILGTVSWNLREMLAHSGGGGSGGGGALTGIAVEQDKNTQEVFAQVKQIYKTPPIKDFGGFNFGGGGSGGGGSAGAAAYYVGYLQPRTFLLKYNENGSSGGGSSGGGLPNNTASWFTALTQHGKEDLKFPRGQGVPGGGGSGGGGSKRFDNPVLPFNDGVYFASTEKSNIIRGSSGGGSSGGGFNATRFASVYAWNRKRISNCVADYSVLYNSASFSTFKGGGGSGGGGSAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSCLYRNRDVDTDFVNEFYAYLRKHFGGGGSGGGGSGVKDCVVLHSYFTSDYYQLYSTQLSTDTGVEHVTFFIGGGGSGGGGSICGDSTECSNLLLQYGSFCTQLNRALTGIAVEQDKNTSGGGGSGGGGKEDLKFPRGQGVPINTNSSPDDQIGYYRRATRRIRGG

SEQ ID NO: 180 - protein, 1657 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSEHDYQIGGYTEKWESGVKDCVVLHSYFTSGGGGSGGGGICGDSTECSNLLLQYGSFCTQLNRALTGIAVEQDKNTGSGGGGSGGGGVKDCVVLHSYFTSDYYQLYSTQLSTDTGVEHVTFFIGGGGSGGGGSCLYRNRDVDTDFVNEFYAYLRKHFGGGGSGGGGSDGKMKDLSPRWYFYYLGTGPEAGGSGGGGSGGQGNFKNLREFVFKNIDGYFKIYSKHTPINLVRDLPQGSSGGGSSGGGFGMSRIGMEVTPSGTWLTYTGAIKLDDKDPNFKDQVIGGGGSGGGGSFYSKWYIRVGARKSAPLIELCVDEAGSKSPIQYIDIGNYSGGGGSGGGGLPNNTASWFTALTQHGKEDLKFPRGQGVPGGGGSGGGGSALTGIAVEQDKNTQEVFAQVKQIYKTPPIKDFGGFNFGGGGSGGGGSPSIISNEKQEILGTVSWNLREMLAHSGGGGSGGGGKDFGGFNFSQILPDPSKPSKRSFIEDLLFNKVTLADAGGGGSGGGGSEGNSPFHPLADNKFALTCFSTQFAFACPDGVKHVYQLRARSVSPKLFIGGGGSGGGGSSFNPETNILLNVPLHGTILTRPLLESELVIGAVILRGHLRIAGHHGGGGSGGGGSMFVFLVLLPLVSSQCVNLTTRTQLPPAYTNSFTRGVGGGGSGGGGSFIRQEEVQELYSPIFLIVAAIVFIGGGGSGGGGSYIIKLIFLWLLWPVTLACFVLAAVYRINWGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSMGYINVFAFPFTIYSLLLCRMNGGGGSGGGGSKKRWQLALSKGVHFVCNLLLLSGGGGSGGGGIILFLALITLATCELYHYQECVRGTTVGGGGSGGGGSEDLLFNKVTLADAGFIKQYGDCLGDIAARDLICAQKFGGGGSGGGGSPPAYTNSFTRGVYYPDKVFRSSVLHSTQDLFLPFFSSGGGGSGGGGMLAKALRKVPTDNYITTYPGQGLGGGSGGGGSGAGAAAYYVGYLQPRTFLLKYNENGSSGGGSSGGGLDDKDPNFKDQVILLNKHIDAYKTFPPTEGGGGSGGGGSKRFDNPVLPFNDGVYFASTEKSNIIRGSSGGGSSGGGAFEYYHTTDPSFLGRYMSALNGGGGSGGGGSMESEFRVYSSANNCTFEYVSQPFLMDLEGKQGNFKNLREFVFKGGGGSGGGGSSTQLSTDTGVEHVTFFIYNKIVDEPEGGGGSGGGGSHLRIAGHHLGRCDIKDLPKEITVATSRTLSYYKLGASQRVAGDSGSSGGGSSGGGWKCRSKNPLLYDANYFLCWHTNCYDGGGGSGGGGSKEDLKFPRGQGVPINTNSSPDDQIGYYRRATRRIRGG

SEQ ID NO: 181 - protein, 1048 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLGVKDCVVLHSYFTSDYYQLYSTQLSSGGGGSGGGGKLPDDFTGCVIAWNSNNLDSKVGGNYNYLYRLFRKSNLKPFERDISTEIYQAGSTPCNGVEGGGGSGGGGSDGKMKDLSPRWYFYYLGTGPEAGGGGSGGGGSVEGFNCYFPLQSYGFQPTNGVGGGGSGGGGSFGMSRIGMEVTPSGTWLTYTGAIKLDDKDPNFKDQVILLNKHIDAYKTFPPTEGGGGSGGGGSPSIISNEKQEILGTVSWNLREMLAHGGSGGGGSGGEGNSPFHPLADNKFALTCFSTQFAFACPDGVKHVYQLRARSVSPKLFIGGGGSGGGGSMGYINVFAFPFTIYSLLLCRMNGGGGSGGGGSMIELSLIDFYLCFLAFLLFLVLIMLIIFWFSSGGGGSGGGGLLLVAAGLEAPFLYLYALVYFLQSINFVRIIMRLWLCWKGGGGSGGGGSIILFLALITLATCELYHYQECVRGTTVGGGGSGGGGSKDFGGFNFSQILPDPSKPSKRSFIEDLLFNKVTLADAGFIKQYGDCLGDIAARDLICAQKFGGGGSGGGGSQTSNFRVQPTESIVRFPNITNLCPFGEVFNATRFASVYAWNRKRISNCVADYSVLYNSASFSTFKGGGGSGGGGSKRFDNPVLPFNDGVYFASTEKSNIIRGSSGGGSSGGGAFEYYHTTDPSFLGRYMSALNGGGGSGGGGSHLRIAGHHLGRCDIKDLPKEITVATSRTLSYYKLGASQRVAGDSGSSGGGSSGGGLPNNTASWFTALTQHGKEDLKFPRGQGVPINTNSSPDDQIGYYRRATRRIRGG

SEQ ID NO: 182 - protein, 1387 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMSFNPETNILLNVPLHGTILTRPLLESELVIGAVILRGHLRIAGHHSGSSGSKDFGGFNFSQILPDPSKPSKRSFIEDLLFNKVTLADAGFIKQYGDCLGDIAARDLICAQKFSGSSGSSTQLSTDTGVEHVTFFIYNKIVDEPESGSSGSMESEFRVYSSANNCTFEYVSQPFLMDLEGKQGNFKNLREFVFKNIDGYFKIYSKHTPINLVRDLPQGSGSSGSAFEYYHTTDPSFLGRYMSALNSGSSGSLLLVAAGLEAPFLYLYALVYFLQSINFVRIIMRLWLCWKSGSSGSFIRQEEVQELYSPIFLIVAAIVFISGSSGSSEHDYQIGGYTEKWESGVKDCVVLHSYFTSDYYQLYSTQLSTDTGVEHVTFFIGGSGGYIIKLIFLWLLWPVTLACFVLAAVYRINWGGSGGCLYRNRDVDTDFVNEFYAYLRKHFGGSGGKRFDNPVLPFNDGVYFASTEKSNIIRGSGSSGSFYSKWYIRVGARKSAPLIELCVDEAGSKSPIQYIDIGNYGGSGGMIELSLIDFYLCFLAFLLFLVLIMLIIFWFSGGSGGICGDSTECSNLLLQYGSFCTQLNRALTGIAVEQDKNTQEVFAQVKQIYKTPPIKDFGGFNFSGSSGSHLRIAGHHLGRCDIKDLPKEITVATSRTLSYYKLGASQRVAGDSGGGSGGDGKMKDLSPRWYFYYLGTGPEAGGSGGPSIISNEKQEILGTVSWNLREMLAHGGSGGWKCRSKNPLLYDANYFLCWHTNCYDGGSGGKKRWQLALSKGVHFVCNLLLLSGSSGSMLAKALRKVPTDNYITTYPGQGLSGSSGSMGYINVFAFPFTIYSLLLCRMNGGSGGMFVFLVLLPLVSSQCVNLTTRTQLPPAYTNSFTRGVYYPDKVFRSSVLHSTQDLFLPFFSGGSGGAGAAAYYVGYLQPRTFLLKYNENGGGSGGEGNSPFHPLADNKFALTCFSTQFAFACPDGVKHVYQLRARSVSPKLFIGGSGGFGMSRIGMEVTPSGTWLTYTGAIKLDDKDPNFKDQVILLNKHIDAYKTFPPTESGSSGSIILFLALITLATCELYHYQECVRGTTVSGSSGSLPNNTASWFTALTQHGKEDLKFPRGQGVPINTNSSPDDQIGYYRRATRRIRGGSGSSGS

SEQ ID NO: 183 - protein, 1515 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMCLYRNRDVDTDFVNEFYAYLRKHFSGSSGSKDFGGFNFSQILPDPSKPSKRSFIEDLLFNKVTLADASGSSGSAFEYYHTTDPSFLGRYMSALNGGSGGEDLLFNKVTLADAGFIKQYGDCLGDIAARDLICAQKFGGSGGICGDSTECSNLLLQYGSFCTQLNRALTGIAVEQDKNTGGSGGDGKMKDLSPRWYFYYLGTGPEAGGSGGKRFDNPVLPFNDGVYFASTEKSNIIRGSGSSGSMESEFRVYSSANNCTFEYVSQPFLMDLEGKQGNFKNLREFVFKSGSSGSMIELSLIDFYLCFLAFLLFLVLIMLIIFWFSSGSSGSFIRQEEVQELYSPIFLIVAAIVFISGSSGSKKRWQLALSKGVHFVCNLLLLSGSSGSIILFLALITLATCELYHYQECVRGTTVGGSGGMFVFLVLLPLVSSQCVNLTTRTQLPPAYTNSFTRGVGGSGGSFNPETNILLNVPLHGTILTRPLLESELVIGAVILRGHLRIAGHHGGSGGMLAKALRKVPTDNYITTYPGQGLSGSSGSLLLVAAGLEAPFLYLYALVYFLQSINFVRIIMRLWLCWKGGSGGLDDKDPNFKDQVILLNKHIDAYKTFPPTEGGSGGYIIKLIFLWLLWPVTLACFVLAAVYRINWSGSSGSQGNFKNLREFVFKNIDGYFKIYSKHTPINLVRDLPQGSGSSGSPPAYTNSFTRGVYYPDKVFRSSVLHSTQDLFLPFFSSGSSGSGVKDCVVLHSYFTSDYYQLYSTQLSTDTGVEHVTFFISGSSGSALTGIAVEQDKNTQEVFAQVKQIYKTPPIKDFGGFNFGGSGGAGAAAYYVGYLQPRTFLLKYNENGSGSSGSFYSKWYIRVGARKSAPLIELCVDEAGSKSPIQYIDIGNYGGSGGWKCRSKNPLLYDANYFLCWHTNCYDGGSGGMGYINVFAFPFTIYSLLLCRMNSGSSGSLPNNTASWFTALTQHGKEDLKFPRGQGVPGGSGGFGMSRIGMEVTPSGTWLTYTGAIKLDDKDPNFKDQVISGSSGSEGNSPFHPLADNKFALTCFSTQFAFACPDGVKHVYQLRARSVSPKLFIGGSGGSEHDYQIGGYTEKWESGVKDCVVLHSYFTSGSSGSKEDLKFPRGQGVPINTNSSPDDQIGYYRRATRRIRGGGGSGGSTQLSTDTGVEHVTFFIYNKIVDEPEGGSGGHLRIAGHHLGRCDIKDLPKEITVATSRTLSYYKLGASQRVAGDSGGGSGGPSIISNEKQEILGTVSWNLREMLAHSGSSGS

SEQ ID NO: 184 - protein, 1067 aa, Synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMFGMSRIGMEVTPSGTWLTYTGAIKLDDKDPNFKDQVIGGSGGQTSNFRVQPTESIVRFPNITNLCPFGEVFNATRSGSSGSMGYINVFAFPFTIYSLLLCRMNGGSGGFNATRFASVYAWNRKRISNCVADYSVLYNSASFSTFKSGSSGSNLDSKVGGNYNYLYRLFRKSNLKPFERDISTEIYQAGSTPCNGVESGSSGSVEGFNCYFPLQSYGFQPTNGVGGSGGLDDKDPNFKDQVILLNKHIDAYKTFPPTESGSSGSGVKDCVVLHSYFTSDYYQLYSTQLSSGSSGSEGNSPFHPLADNKFALTCFSTQFAFACPDGVKHVYQLRARSVSPKLFISGSSGSDGKMKDLSPRWYFYYLGTGPEASGSSGSMIELSLIDFYLCFLAFLLFLVLIMLIIFWFSGGSGGAFEYYHTTDPSFLGRYMSALNSGSSGSKLPDDFTGCVIAWNSNNLDSKVGGNYNYLGGSGGKRFDNPVLPFNDGVYFASTEKSNIIRGGGSGGLLLVAAGLEAPFLYLYALVYFLQSINFVRIIMRLWLCWKGGSGGEDLLFNKVTLADAGFIKQYGDCLGDIAARDLICAQKFGGSGGIILFLALITLATCELYHYQECVRGTTVSGSSGSHLRIAGHHLGRCDIKDLPKEITVATSRTLSYYKLGASQRVAGDSGGGSGGLPNNTASWFTALTQHGKEDLKFPRGQGVPGGSGGKDFGGFNFSQILPDPSKPSKRSFIEDLLFNKVTLADASGSSGSKEDLKFPRGQGVPINTNSSPDDQIGYYRRATRRIRGGSGSSGSPSIISNEKQEILGTVSWNLREMLAHGGSGG

SEQ ID NO: 185 - DNA, 3204 bp, Synthetic construct
  source 1..3204: mol_type = other DNA
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgatgttcggcatgtcacggataggtatggaagtcacgccgagtggaacatggctaacttatactggggccataaaacttgatgacaaggatcccaattttaaggaccaagtgatcggcggcagcggtggacagacctccaacttcagagtgcaaccaacagagtctatcgttaggttccctaacattactaacctatgtccctttggagaggtgttcaacgctacacgctcgggaagttctggcagtatgggatatattaacgtcttcgcttttccgttcacgatctatagcctattgctctgtaggatgaacggtggttccggtggctttaacgcgacacgattcgcgtcggtctatgcatggaaccgtaaacgcatctctaactgcgttgcagattatagtgtgctctacaacagcgcgtccttcagtacctttaagtcgggcagctcagggagcaacctcgacagtaaagtaggtggtaattataactacctgtatcgcttattccgcaagagtaatctcaagccgtttgagcgagacatatccacggaaatctaccaagctgggtctaccccatgtaatggcgtagaatcaggctcttcaggttcggtcgaaggattcaattgttactttcctttacaaagctatggattccagccaactaacggggtgggcggtagtggagggctagacgacaaagaccctaatttcaaagatcaggtgatactcctcaacaagcacattgatgcctacaagacctttccaccaaccgagagtgggtcgtcgggttcgggcgtaaaagattgcgtcgtgttacattcttacttcacgtccgactactaccagttgtactcgactcagcttagtagcggttcttccggcagtgaaggtaactcgccctttcaccctttagccgacaataaattcgcattaacttgttttagtactcaatttgcgttcgcgtgtccagacggagtgaaacatgtgtatcagttacgggcaagaagtgtctctcctaaactattcatctcaggatcatccgggagcgatggcaaaatgaaggacttgagtccacgctggtacttctactatttgggcacaggaccagaagcatcggggagttcagggtccatgatcgaactcagtcttatcgatttctatctatgttttctcgccttccttctatttctagtcctaatcatgctcatcatcttttggttctccggcggttcaggcggagcattcgaatactatcatacaacggacccatcgttcctaggccggtatatgagcgcacttaacagtggctcgtccgggtcaaaactgcctgacgacttcaccgggtgcgtcatcgcctggaactctaataacctggatagcaaagtgggaggcaattacaattacttaggcgggtctggcgggaaacggtttgacaatcccgtgctgccattcaatgatggagtgtatttcgctagtacagaaaagtccaatatcatcaggggcggcggaagtggcggtctgctcttagtggccgctggattagaagcgccgttcttatatctgtatgccctcgtttatttcctgcagtcaatcaactttgtgcggattattatgcggctgtggttgtgttggaagggagggtcgggtggagaagacctgcttttcaataaggtgaccttagctgatgcgggtttcattaagcagtacggcgattgtcttggcgacattgcagctagggatctcatctgtgcgcaaaagtttggaggctccggaggaattatcctcttcctggcccttatcacacttgccacatgcgagctgtaccactatcaggaatgcgtgcgaggcactacagtgtccggtagctctggatcacatctgagaatcgctggccaccacttgggacgctgcgatatcaaggacctacctaaagagatcaccgtggctacctcacgtaccctctcatactacaaactgggcgctagccagagagttgccggggacagcggaggtggatcgggaggcctacccaataataccgcttcatggtttacagcactgacacagcatgggaaagaggacctcaaattcccacgaggccaaggggtgccaggagggtcaggaggtaaagacttcggagggtttaactttagtcagattttacccgatcctagtaagccaagcaaacgctctttcatcgaagatctgctatttaacaaggttacactggctgacgcttcgggatcaagcgggagtaaggaggatcttaagtttcctcgcggacagggtgtgccgataaataccaatagctccccagacgatcagatcggatattacaggcgcgcgacgaggagaattcggggcggctcgggctctagcggctcaccatctattatcagcaatgagaaacaggagattctgggaacagtttcttggaacttgcgtgaaatgctggctcatggcggatctggtgggtaa

SEQ ID NO: 186 - protein, 5 aa, Synthetic construct
GGGGS

SEQ ID NO: 187 - protein, 5 aa, Synthetic construct
GGGSS

SEQ ID NO: 188 - protein, 5 aa, Synthetic construct
GGGSG

SEQ ID NO: 189 - protein, 5 aa, Synthetic construct
GGSGG

SEQ ID NO: 190 - protein, 6 aa, Synthetic construct
SGSSGS

SEQ ID NO: 191 - protein, 10 aa, Synthetic construct
GGGGSGGGGS

SEQ ID NO: 192 - protein, 5 aa, Synthetic construct
GGGGS

SEQ ID NO: 193 - protein, 5 aa, Synthetic construct
  REPEAT 1..5: note = the sequence can be repeated m times, wherein m is an integer from 1 to 5, e.g., 1, 2, 3, 4, or 5
GGGSS

SEQ ID NO: 194 - protein, 5 aa, Synthetic construct
  REPEAT 1..5: note = the sequence can be repeated m times, wherein m is an integer from 1 to 5, e.g., 1, 2, 3, 4, or 5
GGSGG

SEQ ID NO: 195 - protein, 5 aa, Synthetic construct
  REPEAT 1..5: note = the sequence can be repeated m times, wherein m is an integer from 1 to 5, e.g., 1, 2, 3, 4, or 5
GGGSG

SEQ ID NO: 196 - protein, 6 aa, Synthetic construct
  REPEAT 1..6: note = the sequence can be repeated m times, wherein m is an integer from 1 to 5, e.g., 1, 2, 3, 4, or 5
SGSSGS

SEQ ID NO: 197 - protein, 5 aa, Synthetic construct
LGGGS

SEQ ID NO: 198 - protein, 5 aa, Synthetic construct
GLGGS

SEQ ID NO: 199 - protein, 5 aa, Synthetic construct
GGLGS

SEQ ID NO: 200 - protein, 5 aa, Synthetic construct
GGGLS

SEQ ID NO: 201 - protein, 5 aa, Synthetic construct
GGGGL

SEQ ID NO: 202 - protein, 5 aa, Synthetic construct
LGGSG

SEQ ID NO: 203 - protein, 5 aa, Synthetic construct
GLGSG

SEQ ID NO: 204 - protein, 5 aa, Synthetic construct
GGLSG

SEQ ID NO: 205 - protein, 5 aa, Synthetic construct
GGGLG

SEQ ID NO: 206 - protein, 5 aa, Synthetic construct
GGGSL

SEQ ID NO: 207 - protein, 5 aa, Synthetic construct
LGGSS

SEQ ID NO: 208 - protein, 5 aa, Synthetic construct
GLGSS

SEQ ID NO: 209 - protein, 5 aa, Synthetic construct
GGLSS

SEQ ID NO: 210 - protein, 5 aa, Synthetic construct
LGLGS

SEQ ID NO: 211 - protein, 5 aa, Synthetic construct
GLGLS

SEQ ID NO: 212 - protein, 5 aa, Synthetic construct
GLLGS

SEQ ID NO: 213 - protein, 5 aa, Synthetic construct
LGGLS

SEQ ID NO: 214 - protein, 5 aa, Synthetic construct
GLGGL

SEQ ID NO: 215 - protein, 5 aa, Synthetic construct
LGLSG

SEQ ID NO: 216 - protein, 5 aa, Synthetic construct
GLLSG

SEQ ID NO: 217 - protein, 5 aa, Synthetic construct
GGLSL

SEQ ID NO: 218 - protein, 5 aa, Synthetic construct
GGLLG

SEQ ID NO: 219 - protein, 5 aa, Synthetic construct
GLGSL

SEQ ID NO: 220 - protein, 5 aa, Synthetic construct
LGLSS

SEQ ID NO: 221 - protein, 5 aa, Synthetic construct
GGLLS

SEQ ID NO: 222 - protein, 10 aa, Synthetic construct
LGGGSGGGGS

SEQ ID NO: 223 - protein, 10 aa, Synthetic construct
GLGGSGGGGS

SEQ ID NO: 224 - protein, 10 aa, Synthetic construct
GGLGSGGGGS

SEQ ID NO: 225 - protein, 10 aa, Synthetic construct
GGGLSGGGGS

SEQ ID NO: 226 - protein, 10 aa, Synthetic construct
GGGGLGGGGS

SEQ ID NO: 227 - protein, 10 aa, Synthetic construct
LGGSGGGGSG

SEQ ID NO: 228 - protein, 10 aa, Synthetic construct
GLGSGGGGSG

SEQ ID NO: 229 - protein, 10 aa, Synthetic construct
GGLSGGGGSG

SEQ ID NO: 230 - protein, 10 aa, Synthetic construct
GGGLGGGGSG

SEQ ID NO: 231 - protein, 10 aa, Synthetic construct
GGGSLGGGSG

SEQ ID NO: 232 - protein, 10 aa, Synthetic construct
LGGSSGGGSS

SEQ ID NO: 233 - protein, 10 aa, Synthetic construct
GLGSSGGGSS

SEQ ID NO: 234 - protein, 10 aa, Synthetic construct
GGLSSGGGSS

SEQ ID NO: 235 - protein, 10 aa, Synthetic construct
GGGLSGGGSS

SEQ ID NO: 236 - protein, 10 aa, Synthetic construct
GGGSLGGGSS

SEQ ID NO: 237 - protein, 10 aa, Synthetic construct
LGGGSLGGGS

SEQ ID NO: 238 - protein, 10 aa, Synthetic construct
GLGGSGLGGS

SEQ ID NO: 239 - protein, 10 aa, Synthetic construct
GGLGSGGLGS

SEQ ID NO: 240 - protein, 10 aa, Synthetic construct
GGGLSGGGLS

SEQ ID NO: 241 - protein, 10 aa, Synthetic construct
GGGGLGGGGL

SEQ ID NO: 242 - protein, 10 aa, Synthetic construct
LGGSGLGGSG

SEQ ID NO: 243 - protein, 10 aa, Synthetic construct
GLGSGGLGSG

SEQ ID NO: 244 - protein, 10 aa, Synthetic construct
GGLSGGGLSG

SEQ ID NO: 245 - protein, 10 aa, Synthetic construct
GGGLGGGGLG

SEQ ID NO: 246 - protein, 10 aa, Synthetic construct
GGGSLGGGSL

SEQ ID NO: 247 - protein, 10 aa, Synthetic construct
LGGSSLGGSS

SEQ ID NO: 248 - protein, 10 aa, Synthetic construct
GLGSSGLGSS

SEQ ID NO: 249 - protein, 10 aa, Synthetic construct
GGLSSGGLSS

SEQ ID NO: 250 - protein, 6 aa, Synthetic construct
GSGGGA

SEQ ID NO: 251 - protein, 12 aa, Synthetic construct
GSGGGAGSGGGA

SEQ ID NO: 252 - protein, 18 aa, Synthetic construct
GSGGGAGSGGGAGSGGGA

SEQ ID NO: 253 - protein, 24 aa, Synthetic construct
GSGGGAGSGGGAGSGGGAGSGGGA

SEQ ID NO: 254 - protein, 10 aa, Synthetic construct
GENLYFQSGG

SEQ ID NO: 255 - protein, 10 aa, Synthetic construct
SGGGSSGGGS

SEQ ID NO: 256 - protein, 10 aa, Synthetic construct
SSGGGSSGGG

SEQ ID NO: 257 - protein, 10 aa, Synthetic construct
GGSGGGGSGG

SEQ ID NO: 258 - protein, 10 aa, Synthetic construct
GSGSGSGSGS

SEQ ID NO: 259 - protein, 10 aa, Synthetic construct
GGGSSGGGSG

SEQ ID NO: 260 - protein, 6 aa, Synthetic construct
GGGSSS

SEQ ID NO: 261 - protein, 15 aa, Synthetic construct
GGGSSGGGSSGGGSS

SEQ ID NO: 262 - protein, 8 aa, Synthetic construct
GLGGLAAA

SEQ ID NO: 263 - protein, 12 aa, Synthetic construct
KPEPKPAPAPKP

SEQ ID NO: 264 - protein, 12 aa, Synthetic construct
AEAAAKEAAAKA

SEQ ID NO: 265 - protein, 5 aa, Synthetic construct
  REPEAT 1..5: note = the sequence can be repeated m times, wherein m is an integer from 1 to 5, e.g., 1, 2, 3, 4, or 5
EAAAK

SEQ ID NO: 266 - protein, 15 aa, Synthetic construct
PSRLEEELRRRLTEP

SEQ ID NO: 267 - protein, 8 aa, Synthetic construct
SACYCELS

SEQ ID NO: 268 - protein, 16 aa, Synthetic construct
TQKSLSLSPGKGLGGL

SEQ ID NO: 269 - protein, 13 aa, Synthetic construct
SLSLSPGKGLGGL

SEQ ID NO: 270 - protein, 36 aa, Synthetic construct
GGSAGGSGSGSSGGSSGASGTGTAGGTGSGSGTGSG

SEQ ID NO: 271 - protein, 36 aa, Synthetic construct
GGSGGGSEGGGSEGGGSEGGGSEGGGSEGGGSGGGS

SEQ ID NO: 272 - protein, 12 aa, Synthetic construct
ELKTPLGDTTHT

SEQ ID NO: 273 - protein, 15 aa, Synthetic construct
EPKSCDTPPPCPRCP

SEQ ID NO: 274 - DNA, 36 bp, Synthetic construct
  source 1..36: mol_type = other DNA
tggcccaggttccacagcagcgtgttccacacccac

SEQ ID NO: 275 - DNA, 324 bp, Synthetic construct
  source 1..324: mol_type = other DNA
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 276 - protein, 22 aa, Synthetic construct
MQVSTAALAVLLCTMALCNQFS

SEQ ID NO: 277 - protein, 27 aa, Synthetic construct
GYIPEAPRDGQAYVRKDGEWVLLSTFL

SEQ ID NO: 278 - DNA, 117 bp, Synthetic construct
  source 1..117: mol_type = other DNA
aagcctctggacggagagtatttcactctccagatccggggccccgaaaggttcgaaatgttccgggagcttaacgaggccttggagctgaaagacgcacaggccggaaaggaaccg

SEQ ID NO: 279 - DNA, 333 bp, Synthetic construct
  source 1..333: mol_type = other DNA
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 280 - DNA, 81 bp, Synthetic construct
  source 1..81: mol_type = other DNA
gagctcaaaaccccacttggtgacacaactcacacagagcccaaatcttgtgacacacctcccccgtgcccaaggtgccca

SEQ ID NO: 281 - DNA, 321 bp, Synthetic construct
  source 1..321: mol_type = other DNA
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 282 - DNA, 432 bp, Synthetic construct
  source 1..432: mol_type = other DNA
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 283 - protein, 5 aa, Synthetic construct
GLSGL

SEQ ID NO: 284 - protein, 5 aa, Synthetic construct
EAAAK

SEQ ID NO: 285 - protein, 7 aa, Synthetic construct
  REPEAT 1..5: note = the sequence can be repeated m times, wherein m is an integer from 1 to 5, e.g., 1, 2, 3, 4, or 5
EAAAKGS

SEQ ID NO: 286 - protein, 6 aa, Synthetic construct
  REPEAT 1..4: note = the sequence can be repeated m times, wherein m is an integer from 1 to 5, e.g., 1, 2, 3, 4, or 5
EAAKGS

SEQ ID NO: 287 - protein, 17 aa, Synthetic construct
GPSRLEEELRRRLTEPG

SEQ ID NO: 288 - protein, 12 aa, Synthetic construct
HEYGAEALERAG